SEQUENCE LISTING (2 sequences)

SEQ ID NO: 1 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0099607 A1
(43) Pub. Date: Mar. 27, 2025

(54) ANTIBODY DRUG CONJUGATES

(71) Applicant: BeiGene, Ltd., Grand Cayman (KY)

(72) Inventors: Charng-Sheng Tsai, Shanghai (CN); Mei-Hsuan Tsai, Shanghai (CN); Xiaodong Wei, Shanghai (CN)

(73) Assignee: BeiGene, Ltd., Grand Cayman (KY)

(21) Appl. No.: 18/971,958

(22) Filed: Dec. 6, 2024

Related U.S. Application Data
(63) Continuation of application No. PCT/CN23/99148, filed on Jun. 8, 2023.

(30) Foreign Application Priority Data
Jun. 9, 2022 (WO) PCT/CN22/97834

(51) Int. Cl. A61K 47/68; A61P 35/00

(52) U.S. Cl. CPC A61K 47/6889; A61K 47/68031; A61K 47/68037; A61K 47/6849; A61K 47/6851; A61P 35/00

(57) ABSTRACT

Antibody-drug conjugate compounds comprising a linker and methods of using such compounds are provided.

### 1. CROSS-REFERENCE TO RELATED APPLICATIONS

This application is a continuation of International Application No. PCT/CN2023/099148, filed on Jun. 8, 2023, which claims priority to International Application No. PCT/CN2022/097834, filed on Jun. 9, 2022, the disclosures of which are hereby incorporated by reference in their entireties.

### 2. SEQUENCE LISTING

This application contains a Sequence Listing, which has been submitted electronically in XML format. The XML file is entitled “01368-0066-00US.xml”, was created on Dec. 3, 2024, and is 3411 bytes in size. The Sequence Listing is incorporated herein by reference in its entirety.

### 3. FIELD

Provided herein are novel proteins, e.g., antibodies, drug conjugates comprising hydrophilic solubilizing groups and/or linkers comprising hydrophilic solubilizing groups, and methods for treating diseases, disorders, and conditions comprising administering the protein drug conjugates comprising hydrophilic solubilizing groups and/or linkers thereof.

### 4. BACKGROUND

Antibody-drug conjugates (ADCs) are antibodies that are operably linked to a biologically active small molecule, also known as a toxin or payload. ADCs deliver a potent payload selectively to target-expressing cells, leading to a potential reduction of off-target side effects and/or toxicity and improved therapeutic index. The lipophilic nature of many payloads (i.e., drugs) can adversely affect the properties of the ADC to the extent that the payloads are not efficiently delivered to the target cells. Low bioavailability of lipophilic payloads can narrow therapeutic windows for ADC treatment. Furthermore, the hydrophobic nature of payloads can present challenges to their conjugation to antibodies, a reaction performed in aqueous conditions. Thus, there is an ongoing need for the development of hydrophilic linkers for protein conjugates, e.g., ADCs, which would allow for the feasibility of conjugating lipophilic payloads, improved modulation of biological targets, improved bioavailability, and improved therapeutic window.

Monoclonal antibody (mAb) therapies are gaining momentum as adjunct and front-line treatments for cancer. Successes of mAb therapies like AVASTIN™ (anti-VEGF) for colon cancer, RITUXAN™ (Rituximab; anti-CD20) for Non-Hodgkin's Lymphoma and HERCEPTIN™ (anti-Her2) for breast cancer have demonstrated that unconjugated antibodies can improve patient survival without the incidence of significantly increased toxicity.

Monoclonal antibodies can be conjugated with a therapeutic agent to form an antibody-drug conjugate. For example, the HERCEPTIN™ antibody mentioned above was conjugated with a maytansine payload to form the ADC KADCYLA™. ADCs can exhibit increased efficacy, as compared to an unconjugated antibody. The linkage of the antibody to the drug can be direct, or indirect via a linker. The linker can be cleavable or non-cleavable. One of the components believed to be important for developing effective and well-tolerated ADCs is the composition and stability of the linker. For some types of ADCs, the linker desirably provides serum stability, yet selectively releases the drug within the target cell.

Attachment of a linker to a mAb can be accomplished in a variety of ways, such as through surface lysines, reductive coupling to oxidized carbohydrates, and through cysteine residues liberated by reducing interchain disulfide linkages. A variety of ADC linkage systems have been described in the literature, including hydrazone, disulfide, and peptide-based linkages. Some hydrazone and disulfide-based linkers can be labile in circulation, resulting in the undesired release of the drug outside the targeted tissue. It is believed that this premature release of drug can lead to systemic toxicity or organ-specific toxicity and/or less than optimal therapeutic efficacy. Peptide-based linker strategies can provide linkers of higher stability; however, the increased associated hydrophobicity of some linkers can lead to aggregation, particularly with strongly hydrophobic drugs. Such aggregation can lead to a number of undesired effects such as precipitation of the ADC, difficulty in administration, and non-specific uptake of the ADCs into non-targeted tissues, potentially affecting non-target toxicity and reducing efficacy.

Exatecan is a drug which is a structural analog of camptothecin with antineoplastic activity. See Abou-Alfa et al., “Randomized Phase III Study of Exatecan and Gemcitabine Compared with Gemcitabine Alone in Untreated Advanced Pancreatic Cancer,” Journal of Clinical Oncology, 24 (27): 4441-7, Sep. 20, 2006. Monomethyl auristatin E (MMAE) is a synthetic antineoplastic agent. Because of its toxicity, it cannot be used as a drug itself. MMAE is actually desmethyl-auristatin E; that is, the N-terminal amino group has only one methyl substituent instead of two as in auristatin E itself. See Dosio et al., “Immunotoxins and Anticancer Drug Conjugate Assemblies: The Role of the Linkage between Components,” Toxins. 3 (12): 848-883, 2011.

In the realm of small molecule therapeutics, strategies have been developed to provide prodrugs of an active chemical entity. Such prodrugs are administered in a relatively inactive (or significantly less active) form. Once administered, the prodrug is metabolized in vivo into the active compound. Such prodrug strategies can provide for increased selectivity of the drug for its intended target and for a reduction of adverse effects.

There remains a need, therefore, for targeted delivery of toxins, resulting in the elimination of targeted cells while reducing toxicity to non-target cells.

Furthermore, some antibody, such as Patritumab, has visually observable aggregation during rapid buffer exchange. Its aggregation temperature (Tagg) detected by dynamic light scattering (DLS) and self-association tendency detected by the AC-SINS assay are both worse than those of a panel of well-behaved mAbs. The aggregation tendency of patritumab results in the aggregation of the corresponding ADCs.

There is therefore an unmet medical need to create ADCs with linker systems that provide a high level of linker serum stability and increased solubility, allowing the efficient conjugation of hydrophobic drugs and the effective intracellular delivery of drugs.

### 5. BRIEF SUMMARY

Provided herein are ADCs with linker systems that provide a high level of linker serum stability and increased solubility, allowing the efficient conjugation of hydrophobic drugs and the effective intracellular delivery of drugs.

In one embodiment, the ADC is a compound of Formula (I):

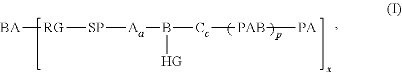

or a pharmaceutically acceptable salt, tautomer, solvate, stereoisomer, enantiomer, isotopologue, or prodrug thereof, wherein the variables are defined as herein. In one embodiment, the ADC comprises one or more hydrophilic residues.

In another embodiment, the ADC is a compound of Formula (Ia):

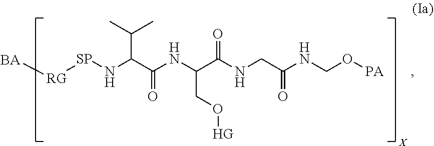

or a pharmaceutically acceptable salt, tautomer, solvate, stereoisomer, enantiomer, isotopologue, or prodrug thereof, wherein the variables are defined as herein.

In another embodiment, the ADC is a compound of Formula (Ib):

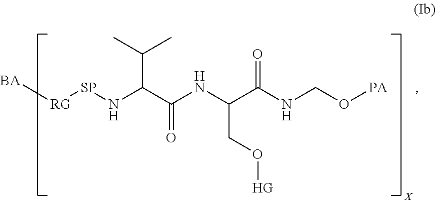

or a pharmaceutically acceptable salt, tautomer, solvate, stereoisomer, enantiomer, isotopologue, or prodrug thereof, wherein the variables are defined as herein.

In another embodiment, set forth herein is a method of treating a disease, condition, or disorder in a patient in need thereof including administering to the patient a compound set forth herein. Also provided is the use of a compound set forth herein for treating a disease, condition, or disorder set forth herein. Further provided is the use of a compound set forth herein for the manufacture of a medicament for treating a disease, condition, or disorder set forth herein. In some embodiments, the compound is an antibody-drug conjugate.

In another embodiment, set forth herein is a method of preparing an antibody-drug conjugate including the step of contacting a binding agent with a linker-payload compound set forth herein under conditions suitable for forming a bond between the binding agent and the linker-payload compound.

### 6. BRIEF DESCRIPTION OF FIGURES

FIG. 1 illustrates the tandem release fashion of ADCs.

FIG. 2, FIG. 3, FIG. 4, and FIG. 5 illustrate that, with tandem release linker design, the cellular killings of ADC-C10, ADC-C11, ADC-C13, and ADC-C14 appear to be similar to ADC-C1 on H358, H441, H1048 and MDA-MB-453 cell lines, indicating efficient payload release within cells.

FIG. 6, FIG. 7, FIG. 8, and FIG. 9 illustrate that, with tandem release linker design, the cellular killings of ADC-C3, ADC-C4, ADC-C5, and ADC-C6 appear to be similar to ADC-C1 on H358, H441, H1048 and MDA-MB-453 cell lines, indicating efficient payload release within cells.

FIG. 10, FIG. 11, FIG. 12, and FIG. 13 illustrate that, with tandem release linker design, the cellular killings of ADC-C7, ADC-C8, ADC-C9, and ADC-C12 appear to be similar to ADC-C1 on H358, H441, H1048 and MDA-MB-453 cell lines, indicating efficient payload release within cells.

FIG. 14 illustrates the payload release profile of ADC-C10, ADC-C11, ADC-C13, and ADC-C14 under lysosome treatment.

FIG. 15 illustrates the payload release profile of ADC-C10, ADC-C11, ADC-C13, and ADC-C14 under Cathepsin B treatment.

FIG. 16 illustrates the payload release profile of ADC-C3, ADC-C4, ADC-C5, and ADC-C6 under lysosome treatment.

FIG. 17 illustrates the payload release profile of ADC-C3, ADC-C4, ADC-C5, and ADC-C6 under Cathepsin B treatment.

FIG. 18 illustrates the payload release profile of ADC-C7, ADC-C8, ADC-C9, and ADC-C12 under lysosome treatment.

FIG. 19 illustrates the payload release profile of ADC-C7, ADC-C8, ADC-C9, and ADC-C12 under Cathepsin B treatment.

FIG. 20 illustrates the payload release profile of ADC-C10, ADC-C11, ADC-C13, and ADC-C14 under human plasma treatment, showing comparable and acceptable payload release rate.

FIG. 21 illustrates the payload release profile of ADC-C3, ADC-C4, ADC-C5, and ADC-C6 under human plasma treatment, showing comparable and acceptable payload release rate.

FIG. 22 illustrates the payload release profile of ADC-C7, ADC-C8, ADC-C9, and ADC-C12 under human plasma treatment, showing comparable and acceptable payload release rate.

### 7. DETAILED DESCRIPTION

Provided herein are compounds, compositions, ADCs, and methods useful for treating a wide variety of human cancers, including, but not limited to, colorectal cancer, gastric cancer, breast cancer, non-small cell lung cancer (NSCLC), ovarian cancer, head and neck cancer, pancreatic cancer and cervical cancer. In one embodiment, provided herein are compounds, compositions, ADCs, and methods useful for treating a wide variety of human cancers.

Within the present disclosure, it is understood that the disclosure does not limit to particular methods and/or experimental conditions described, as such methods and conditions may vary. It is also to be understood that the terminology used herein is for the purpose of describing particular embodiments only, and is not intended to be limiting,

Although any methods and materials similar or equivalent to those described herein can be used in the practice or testing of the present invention, the preferred methods and materials are now described. All patents, applications and non-patent publications mentioned in this specification are incorporated herein by reference in their entireties.

### 7.1. Definitions

When referring to the compounds provided herein, the following terms have the following meanings unless indicated otherwise. Unless defined otherwise, all technical and scientific terms used herein have the same meaning as is commonly understood by one of ordinary skill in the art to which this invention belongs. In the event that there is a plurality of definitions for a term provided herein, these Definitions prevail unless stated otherwise.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with amounts, or weight percentage of ingredients of a composition, mean an amount, or weight percent that is recognized by one of ordinary skill in the art to provide a pharmacological effect equivalent to that obtained from the specified amount, or weight percent. In certain embodiments, the terms “about” and “approximately,” when used in this context, contemplate an amount, or weight percent within 30%, within 20%, within 15%, within 10%, or within 5%, of the specified amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 20 (or 0.2 degree 20) while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and n-hexyl; while saturated branched alkyls include -isopropyl, -sec-butyl, -isobutyl, -tert-butyl, -isopentyl, 2-methylpentyl, 3methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), C(CH₂CH₃)═CH₂, C≡CH, —C≡C(CH₃), —C≡C(CH₂CH₃), —CH₂C≡CH, —CH₂C≡C(CH₃) and CH₂C≡C(CH₂CH₃), among others. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂C₈)alkenyls include -vinyl, -allyl, -1-butenyl, -2-butenyl, -isobutylenyl, -1-pentenyl, 2pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3octenyl and the like. The double bond of an alkenyl group can be unconjugated or conjugated to another unsaturated group. An alkenyl group can be unsubstituted or substituted.

A “cycloalkyl” group is a saturated, or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3Himidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include but are not limited to 4-ethyl-morpholinyl, 4-propylmorpholinyl, furan-2-yl methyl, furan-3-yl methyl, pyridine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl)O(alkyl), wherein alkyl is defined above.

An “amine” group is a radical of the formula: NH₂.

A “hydroxyl amine” group is a radical of the formula: N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: N(R^{#})O-aryl or NHOaryl, wherein R^{#} is as defined above.

An “alkylamine” group is a radical of the formula: NHalkyl or N(alkyl)₂, wherein each alkyl is independently as defined above.

An “aminocarbonyl” group is a radical of the formula: —C(═O)N(R^{#})₂, —C(═O)NH(R^{#}), or C(═O)NH₂, wherein each R^{#} is as defined above.

An “acylamino” group is a radical of the formula: NHC(═O)(R^{#}) or N(alkyl)C(═O)(R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}) or —O(alkyl)C(═O)NH₂, wherein each R^{#} is independently as defined above.

An “N-oxide” group is a radical of the formula: —N⁺—O⁻.

A “carboxy” group is a radical of the formula: C(═O)OH.

A “ketone” group is a radical of the formula: C(═O)(R^{#}), wherein R^{#} is as defined above.

An “aldehyde” group is a radical of the formula: —CH(═O).

An “ester” group is a radical of the formula: C(═O)O(R^{#}) or OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula: —N═C(R^{#})₂ or —C(R^{#})═N(R^{#}), wherein each R^{#} is independently as defined above.

An “imide” group is a radical of the formula: —C(═O)N(R^{#})C(═O)(R^{#}) or wherein each R^{#} is independently as defined above.

A “urethane” group is a radical of the formula: —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N=C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula: —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), NHC(═NH)NH₂, —N═C(N(R^{#})₂)₂, —N═C(NH(R^{#}))₂, or —N═C(NH₂)₂, wherein each R^{#} is independently as defined above.

An “enamine” group is a radical of the formula: —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula: —N₃.

An “isocyanate” group is a radical of the formula: N═C═O.

An “isothiocyanate” group is a radical of the formula: N═C═S.

A “cyanate” group is a radical of the formula: OCN.

A “thiocyanate” group is a radical of the formula: SCN.

A “thioether” group is a radical of the formula; —S(R^{#}), wherein R^{#} is as defined above.

A “thiocarbonyl” group is a radical of the formula: —C(═S)(R^{#}), wherein R^{#} is as defined above.

A “sulfinyl” group is a radical of the formula: —S(═O)(R^{#}), wherein R^{#} is as defined above.

A “sulfone” group is a radical of the formula: —S(═O)₂(R^{#}), wherein R^{#} is as defined above.

A “sulfonylamino” group is a radical of the formula: —NHSO₂(R^{#}) or —N(alkyl)SO₂(R^{#}), wherein each alkyl and R^{#} are defined above.

A “sulfonamide” group is a radical of the formula: —S(═O)₂N(R^{#})₂, or S(═O)₂NH(R^{#}), or —S(═O)₂NH₂, wherein each R^{#} is independently as defined above.

A “phosphonate” group is a radical of the formula: —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula: —P(R^{#})₂, wherein each R is independently as defined above.

When the groups described herein, with the exception of alkyl group are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, orthiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound, particularly a compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

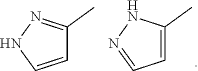

As readily understood by one skilled in the art, a wide variety of functional groups and other structures may exhibit tautomerism and all tautomers of the compounds are within the scope of the present invention.

It should also be noted the compounds can contain unnatural proportions of atomic isotopes at one or more of the atoms. For example, the compounds may be radiolabeled with radioactive isotopes, such as for example tritium (H), iodine-125 (¹²⁵I), sulfur35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

The term “effective amount” in connection with a compound means an amount capable of alleviating, in whole or in part, symptoms, or slowing or halting further progression or worsening of those symptoms. As will be apparent to those skilled in the art, it is to be expected that the effective amount of a compound disclosed herein may vary depending on the severity of the indication being treated.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂-8 alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, and not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

As used herein, “binding agent” refers to any molecule, e.g., antibody, capable of binding with specificity to a given binding partner, e.g., antigen.

As used herein, the term “amino acid” refers to an organic compound that contains amine (—NH₂) and carboxyl (—COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine and citrulline.

As used herein “peptide”, in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including Glycine and both the D or L optical isomers, and amino acid analogs and peptidomimetics. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include postexpression modifications of the polypeptide, for example, glycosylation, acetylation, phosphorylation and the like. Furthermore, as ionizable amino and carboxyl groups are present in the molecule, a particular peptide may be obtained as an acidic or basic salt, or in neutral form. A peptide may be obtained directly from the source organism, or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

As used herein, the term “anti-HER2 antibody” refers to an antibody selectively binding to the HER2 receptor, e.g., trastuzumab. In one embodiment, trastuzumab can be made and used as described in U.S. Pat. Nos. 6,407,213, and 5,821,337, the entire disclosure of which is incorporated herein by reference.

As used herein, the term “anti-HER3 antibody” refers to an antibody selectively binding to the HER3 receptor, e.g., patritumab. In one embodiment, patritumab can be made and used as described in U.S. Pat. No. 7,705,130, the entire disclosure of which is incorporated herein by reference.

As used herein, the term “anti-PTK7 antibody” refers to an antibody selectively binding to the PTK7 receptor, e.g., cofetuzumab. In one embodiment, cofetuzumab can be made and used as described in U.S. Pat. No. 9,777,070, the entire disclosure of which is incorporated herein by reference.

As used herein, the term “Ifinatamab” refers to an antibody selectively binding to the B7H3 receptor. In one embodiment, Ifinatamab can be made and used as described in U.S. Ser. No. 10/117,952 or WO2022102695, the entire disclosure of which is incorporated herein by reference.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

As used herein, the term “cell-killing activity” refers to the activity that decreases or reduces the cell viability of the tested cell line.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

### 7.2. Conjugates

In some other examples, set forth herein is an ADC, or a pharmaceutically acceptable salt thereof, comprising: a protein linked to at least one payload moiety and linked to at least one hydrophilic moiety via a covalent linker, wherein said covalent linker is bonded directly or indirectly to each of the protein, the payload moiety, and the hydrophilic moiety. In some embodiments, the protein is an antibody or antigen binding fragment thereof.

As illustrated herein, in some examples, the binding agent is bonded directly to a covalent linker, such as a linker set forth herein. This means that the binding agent is one bond position away from the covalent linker set forth herein. In some of these examples, the covalent linker is also bonded directly to a payload moiety. This means that the covalent linker is one bond position away from a payload such as, but not limited to, Dxd, MMAE, or a stereoisomer thereof, or any payload set forth herein. In some of these examples, the covalent linker is also bonded directly to a hydrophilic moiety. This means that the covalent linker is one bond position away from a hydrophilic residue, such as the hydrophilic residues set forth herein. In some of these examples, the covalent linker is a covalent linker set forth herein.

In other examples, the binding agent is bonded indirectly to a covalent linker. This means that the binding agent is more than one bond position away from the covalent linker.

This also means that the binding agent is bonded through another moiety to the covalent linker. For example, the binding agent may be bonded to a maleimide group which is bonded to a polyethylene glycol group which is bonded to the covalent linker. In some of these examples, the covalent linker is also bonded indirectly to a payload moiety. This means that the covalent linker is more than one bond position away from a payload such as, but not limited to, Dxd, MMAE, or a stereoisomer thereof, or any payload set forth herein. This also means that the covalent linker is bonded through another moiety to the payload. For example, the covalent linker may be bonded to a dipeptide, such as but not limited to Val-Ala or Val-Cit, which may be bonded to PAB which may be bonded to the payload. In some of these examples, the covalent linker is also bonded indirectly to a hydrophilic moiety. This means that the covalent linker is more than one bond position away from a hydrophilic moiety, such as the hydrophilic residues set forth herein. This also means that the covalent linker is bonded through another moiety to the hydrophilic moiety.

In certain instances, the hydrophilic residue comprises a terminal hydrophilic group. In some instances, the hydrophilic residue comprises at least one sugar residue. In some instances, the hydrophilic residue comprises a sugar residue. In some cases, the hydrophilic residue comprises a terminal sugar residue. In further instances, the hydrophilic residue comprises more than one sugar residue. In some cases, the hydrophilic residue comprises more than one terminal sugar residue.

In another embodiment, the payload provided here is a chromophore functional group, for which the compound provided herein can be used for detection, monitoring, or study the interaction of the cell binding molecule with a target cell. Chromophore functional groups are functional groups that have the ability to absorb a kind of light, such as UV light, florescent light, IR light, near IR light, visual light. A chromatophore functional group is a functional group selected from a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; and a class or subclass of luciferin compounds.

### 7.2.1. Aspect 1

Described herein are compounds according to Formula (I):

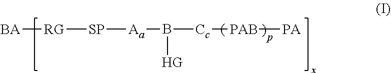

or a pharmaceutically acceptable salt, tautomer, solvate, stereoisomer, enantiomer, isotopologue, or prodrug thereof,
wherein BA is a binding agent selected from a humanized, chimeric, or human antibody or an antigen binding fragment of an antibody;
RG is a reactive group residue; SP is a spacer group residue; each of A, B and C is independently, in each instance, an amino acid residue; HG is a hydrophilic residue or hydrogen; subscripts a, c, and p are independently, in each instance, 0 or 1; PA is a payload residue; subscript x is from 1 to 15; and
PAB represents —NH—CH₂—O—.

In one example, BA is an antibody. In one example, the antibody is a humanized, chimeric, or human antibody or an antigen binding fragment of an antibody. In one example, the antibody is a humanized, chimeric, or human anti-HER2 or anti-HER3 antibody or an antigen binding fragment of an anti-HER2 or anti-HER3 antibody. In one example, the antibody is a monoclonal antibody.

In one example, BA is an antibody. In one example, the antibody is a humanized, chimeric, or human antibody or an antigen binding fragment of Ifinatamab, cofetuzumab, patritumab, or trastuzumab. In one example, the antibody is a humanized, chimeric, or human antibody or an antigen binding fragment of Ifinatamab. In one example, the antibody is Ifinatamab, cofetuzumab, patritumab, or trastuzumab or an antigen binding fragment of Ifinatamab, cofetuzumab, patritumab, or trastuzumab. In one example, the antibody is Ifinatamab or an antigen binding fragment of Ifinatamab.

In certain embodiments, the antibody as described herein binds to one or more of receptors selected from the group consisting of: HER2, HER3, CD7, CD19, CD20, CD22, CD25, CD27, CD30, CD33, CD37, CD38, CD46, CD70, CD71, CD74, CD79b, CD123, CD138, CD142, CD166, CD205, CD228, CCR2, CA6, p-Cadherin, CEA, CEACAM5, C4.4a, DLL3, EGFR, EGFRVIII, ENPP3, EphA2, EphrinA, FLOR1, FGFR2, GCC, cKIT, LIV1, LY6E, MSLN, MUC16, NaPi2b, Nectin4, gpNMB, PSMA, SLITRK6, STEAP1, TROP2, 5T4, SSEA4, GloboH, Gb5, STn, Tn, B7H3, BCMA, MUC1, cMet, ROR1 MSLN, FRa, CLDN18.2, CLDN6, PTK7 and Axl. In certain embodiments, the antibody as described herein binds to one or more of receptors selected from the group consisting of: B7H3, MUC1, FGFR2b, CLL1, CCR7, GPC1, and GPC3.

In certain embodiments, the antibody as described herein binds to CEA receptors. In certain embodiments, the antibody as described herein binds to one or more of receptors selected from the group consisting of: CEACAM1, CEACAM3, CEACAM4, CEACAM5, CEACAM6, CEACAM7, CEACAM8, CEACAM16, CEACAM18, CEACAM19, CEACAM20, and CEACAM21 receptors.

In certain embodiments, the antibody as described herein binds to CEACAM5 receptors.

In certain embodiments, the antibody as described herein binds to CEACAM6 receptors.

In certain embodiments, the antibody as described herein is a bispecific antibody.

In certain embodiments, RG is

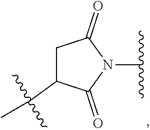 [SMILES: *C1CC(=O)N(*)C1=O]

### -(Succinimid-3-yl-N)—,

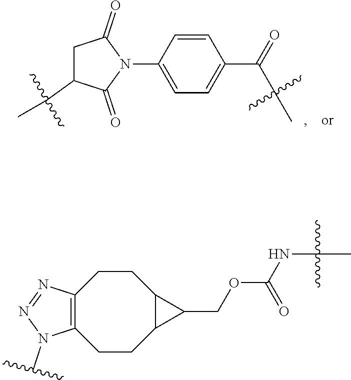 [SMILES: *C(=O)c1ccc(N2C(=O)CC(*)C2=O)cc1 ; *NC(=O)OCC1C2CCc3nnn(*)c3CCC21]

In certain embodiments, RG is

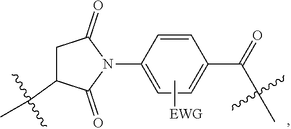

wherein EWG is an electrowithdrawn group selected from —CN, halogen, —CF₃, —C(═O)OR¹, and —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In certain embodiments, RG is

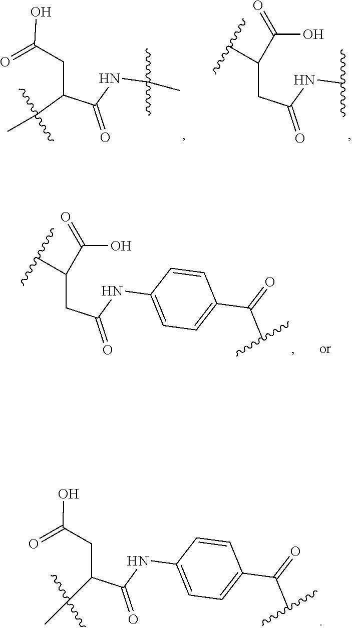 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1]

In certain embodiments, RG is

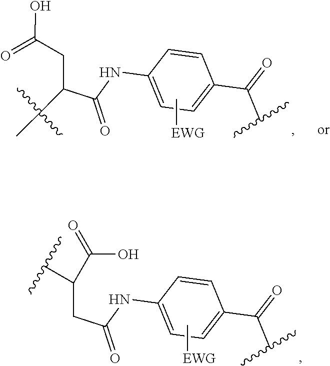

wherein EWG is an electrowithdrawn group selected from —CN, halogen, —CF₃, —C(═O)OR¹, and —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In certain embodiments, SP is —(CH₂)ₙ—C(═O)—, —CH₂—C(═O)—NH—(CH₂)ₙ—C(═O)—, —(CH₂CH₂O)ₙ—CH₂CH₂—C(═O)—, —CH[—(CH₂)ₙ—COOH]—C(═O)—, —CH₂—C(═O)—NH—(CH₂)ₙ—C(═O)—NH—(CH₂)ₙ—C(═O)—, or —C(═O)—(CH₂)ₙ—C(═O)— wherein each of n independently represents an integer of 1 to 8.

In certain embodiments, SP is —(CH₂)ₙ—C(═O)—, or —CH₂—C(═O)—NH—(CH₂)ₙ—C(═O)—; and each of n independently represents an integer of 5 or 2.

In certain embodiments, RG is

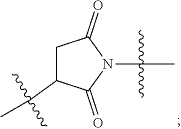 [SMILES: *C1CC(=O)N(*)C1=O]

SP is —(CH₂)ₙ—C(═O)—; and n is an integer of 1 2, 3, 4, or 5; preferably n is an integer of 1, or 2.

In certain embodiments, HG is selected from the group consisting of a saccharide, phosphate ester, sulfate ester, a phosphodiester and a phosphonate.

In certain embodiments, the saccharide is selected from the group consisting of β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid or any analogue or modification thereof, or sulfate, phosphate, carboxyl, amino, or O-acetyl modification thereof; preferably β-D-galactose and b-glucuronic acid.

In certain embodiments, HG is

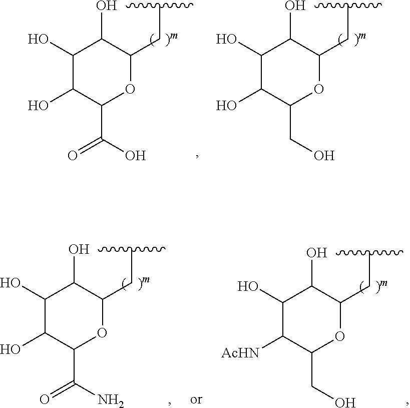

or a stereoisomer, enantiomer, isotopologue, or prodrug thereof, wherein each m is independently 0 or 1.

In certain embodiments, HG is

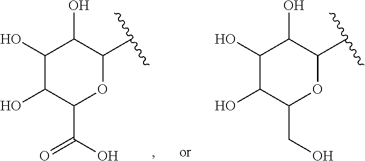 [SMILES: *C1OC(C(=O)O)C(O)C(O)C1O ; *C1OC(CO)C(O)C(O)C1O]

or a stereoisomer, enantiomer, isotopologue, or prodrug thereof.

In certain embodiments, each PA independently represents a chromophore functional group.

In certain embodiments, each chromophore functional group is independently a functional group selected from a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; and a class or subclass of luciferin compounds.

In certain embodiments, each PA is independently a cytotoxic agent, wherein the cytotoxic agent contains at least one hydroxyl group. Examples of the cytotoxic agent include, but are not limited to, 10-Deacetyl-7-xylosyl paclitaxel, 17-AAG, 17-AEP-GA, 17-AH-Geldanamycin, 17-DMAP-GA, 17-GMB-APA-GA, 4-methyl-Ahtea-Puwainaphycin F, 9-Hydroxyellipticine, hydrochloride, Actinomycin X2, Aeroplysinin 1, Aeruginosin 865, Agrochelin A, Agrochelin B, alpha-Amanitin, alpha-Amanitin—fungal fermentation origin, Ansamitocin P-3, Ansatrienin B, Aphidicolin, Apoptolidin, Auristatin E, Auristatin F, AZD4547, Free Base, AZD8055, Free Base, Bafilomycin A1, beta-Amanitin, Boc-Nme-Val-Val-Dil-Dap-OH, Boc-Val-Dil-Dap-Doe, Boc-Val-Dil-Dap-OH, Boc-Val-Dil-Dap-Phe-Ome, Calicheamicin, Campathecin, Chaetocin, Chaetoglobosin, Chaetoglobosin C13, Chlamydocin, Cinerubin B, Colchicine, Combretastatin-A4, Compound CL0485, Cordycepin, Cryptophycin, Cucurbitacin B, Cucurbitacin E, Curvulin, Cyclopamine, Free Base, D8-MMAE, Daun02, Daunorubicin, Daunorubicin HCl, Daunorubicin Hydrochloride, DGN462, DL-Dithiothreitol/DTT, DL-Dithiothreitol/DTT—biotech grade, DL-Dithiothreitol/DTT—pure grade, DM1, DM4, Dolastatin 10, Dolastatin 15, DOXO-EMCH, Doxorubicin, Doxorubicin hydrochloride, Duocarmycin DM, Duocarmycin MA, Duocarmycin™, Englerin A, Epothilone A, Epothilone B, epsilon-Amanitin, Ferulenol, Fumagillin, gamma-Amanitin, Geldanamycin, Glucopiericidin A, Gramicidin A, Herboxidiene, HL-100-AL1-R01 (H-3137), Hygrolidin, Hypothemycin, Ilimaquinone, Isatropolone A, Isofistularin-3, Ixabepilone, Free Base, JW 55, Lactacystin, Luisol A, Maytansine DM3, Maytansinoid AP-3, Maytansinol, Mechercharmycin A, Mensacarcin, Methotrexate, Microcolin B, Microcystin LR, MMAD, MMAD hydrochloride, MMAF, MMAF Hydrochloride, Monomethyl auristatin E, Monomethyl auristatin F methyl ester, Muscotoxin A, Myoseverin, Mytoxin B, N-acetyl Calicheamicin g1, Nemorubicin, Nocuolin A, Okilactomycin, Oligomycin B, Paclitaxel, PF-06380101, Phallacidin, Phalloidin, Phytosphingosine, Piericidin A, Pironetin, PNU-159682, Podophyllotoxin, Polyketomycin, Pseudolaric acid B, Pseurotin A, Puwainaphycin F, Pyrrolobenzodiazepine Dimer, Quinaldopeptin, Rachelmycin, Rebeccamycin, Ro 5-3335, Safracin B, Sandramycin, Sanguinarine, Saporin, Seco-Duocarmycin SA, Sinefungin, Taltobulin, Taltobulin hydrochloride, Taltobulin trifluoroacetate, Telomestatin, Thiocolchicine, Tolytoxin, Tripolin A, Triptolide, Tubastatin A HCl, Tubulysin A, Tubulysin IM-1, Tubulysin IM-2, Tubulysin IM-3, and Tubulysin M. In certain embodiments, each PA is a cytotoxic agent designed to induce target cell death after being internalized in the tumor cell and released. In certain embodiments, each PA is a cytotoxic agent designed to induce target cell death after being internalized in the tumor cell. In certain embodiments, each PA is a small molecule drug with high systemic toxicity.

In certain embodiments, each PA is independently selected from the group consisting of Monomethyl auristatin E (MMAE), Monomethyl auristatin F (MMAF), Monomethyl auristatin D (MMAD), Mertansine (Maytansinoid DM1/DM4), Paclitaxel, Docetaxel, Epothilone B, Epothilone A, CYT997, Auristatin tyramine phosphate, Auristatin aminoquinoline, Halocombstatins, Calicheamicin theta, 7-Ethyl-10-hydroxy-camptothecin (SN-38), Pyrrolobenzodiazepine (PBD), Pancratistatin, Cyclophosphate, Cribrostatin-6, Kitastatin, Turbostatin 1-4, Halocombstatins, Eribulin, Hemiasterlin, PNU and Silstatins.

In certain embodiments, each PA independently represents formula (VI):

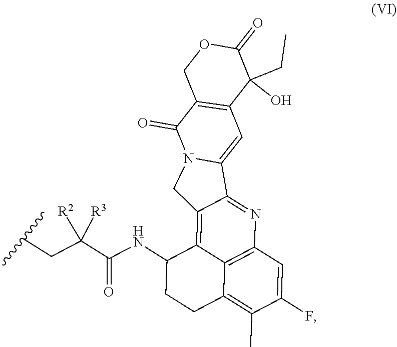

wherein each of R², and R³ is independently hydrogen, halogen, or substituted or unsubstituted C₁₋₄ alkyl.

In one embodiment, R² and R³ are hydrogen.

In one embodiment, R² and R³ are methyl.

In one embodiment, R² is methyl, and R³ is F.

In one embodiment, the carbon that R² and R³ connect to is in the S configuration.

In one embodiment, the carbon that R² and R³ connect to is in the R configuration.

In certain embodiments, each PA independently represents

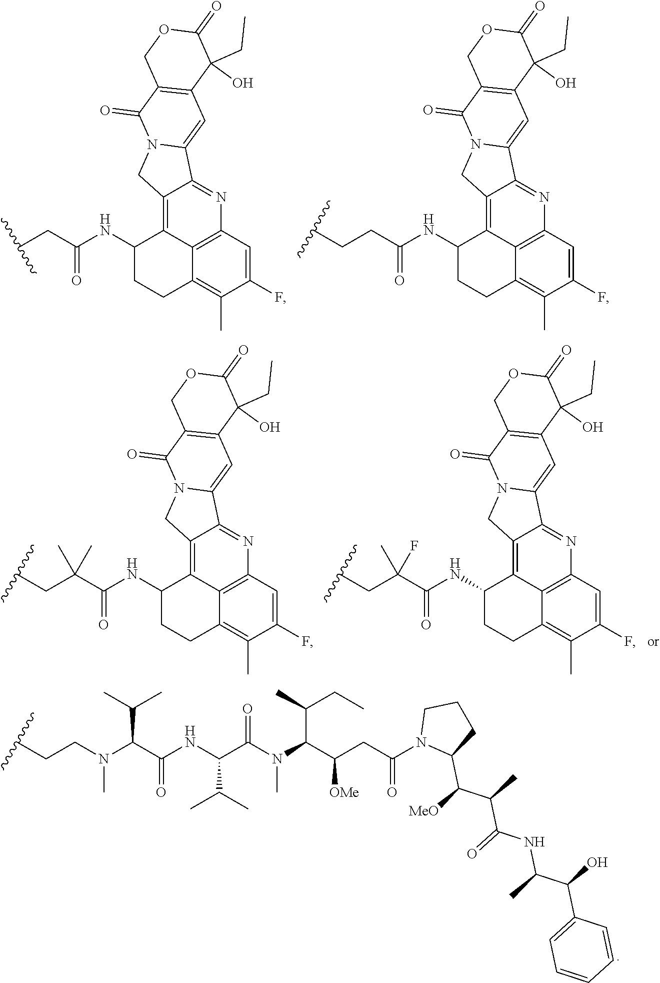

In certain embodiments, each PA independently represents

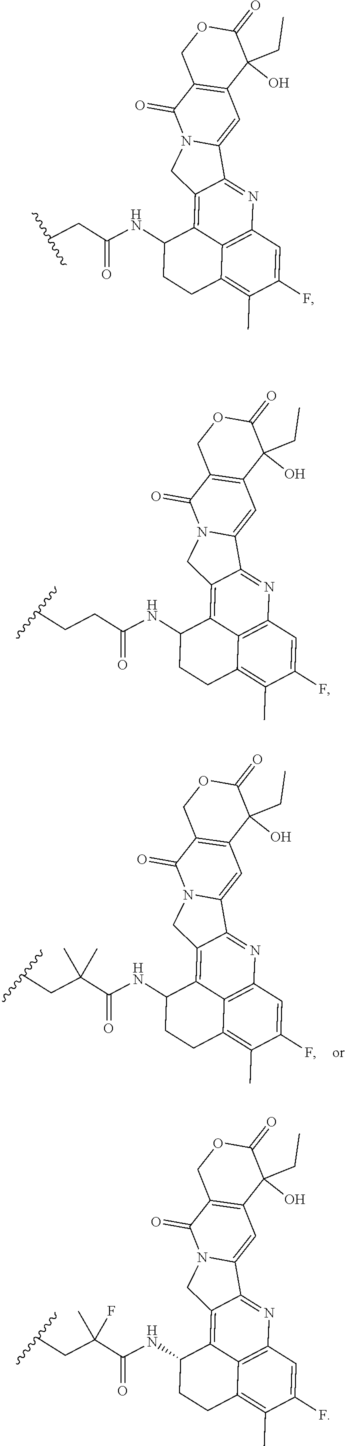

In one embodiment, subscript x is from 2 to 12. In one embodiment, subscript x is from 4 to 12. In one embodiment, subscript x is from 6 to 12. In one embodiment, subscript x is from 8 to 12. In one embodiment, subscript x is from 9 to 11.

### 7.2.2. Aspect 2

Described herein are compounds according to Formula (Ia):

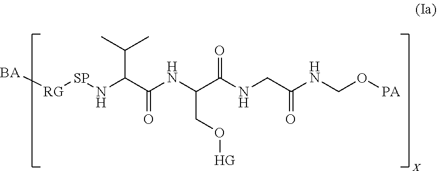

or a pharmaceutically acceptable salt, tautomer, solvate, stereoisomer, enantiomer, isotopologue, or prodrug thereof.

In certain embodiments, the BA, RG, SP, HG, PA, and x are as provided herein.

### 7.2.3. Aspect 3

Described herein are compounds according to Formula (Ib):

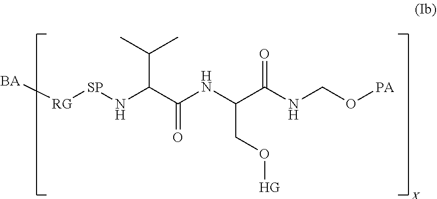

or a pharmaceutically acceptable salt, tautomer, solvate, stereoisomer, enantiomer, isotopologue, or prodrug thereof.

In certain embodiments, the BA, RG, SP, HG, PA, and x are as provided herein.

### 7.2.4. Aspect 4

Described herein are compounds according to Formula (II):

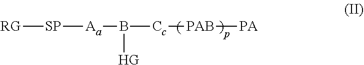

or a pharmaceutically acceptable salt, tautomer, solvate, stereoisomer, enantiomer, isotopologue, or prodrug thereof.

In certain embodiments, RG is

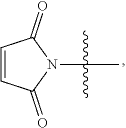 [SMILES: *N1C(=O)C=CC1=O]

### Succinimid-N—,

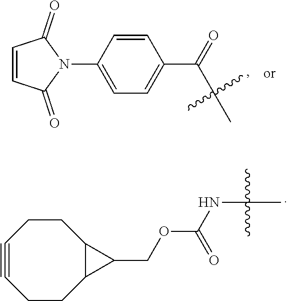 [SMILES: *C(=O)c1ccc(N2C(=O)C=CC2=O)cc1 ; *NC(=O)OCC1C2CCC#CCCC21]

In certain embodiments, RG is

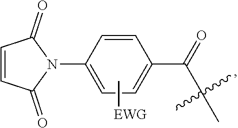

wherein EWG is an electrowithdrawn group selected from the group consisting of —CN, halogen, —CF₃, —C(═O)OR¹, and —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In certain embodiments, RG is

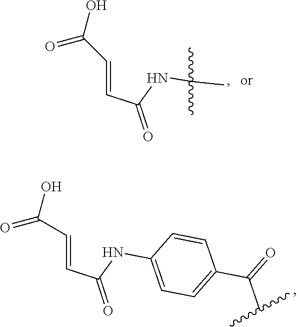 [SMILES: *NC(=O)/C=C/C(=O)O ; *C(=O)c1ccc(NC(=O)/C=C/C(=O)O)cc1]

The In certain embodiments, RG is

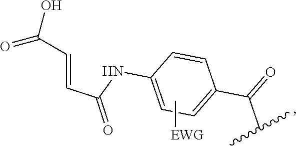

wherein EWG is an electrowithdrawn group selected from —CN, halogen, —CF₃, —C(═O)OR¹, and —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In certain embodiments, the SP, A, B, C, HG, PAB, PA, a, c and p are as provided herein.

### 7.2.5. Aspect 5

Described herein are compounds according to Formula (IIa):

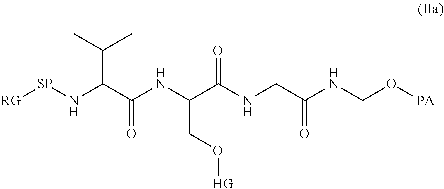

or a pharmaceutically acceptable salt, tautomer, solvate, stereoisomer, enantiomer, isotopologue, or prodrug thereof.

In certain embodiments, RG is

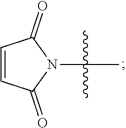 [SMILES: *N1C(=O)C=CC1=O]

SP is —(CH₂)ₙ—C(═O)—; and n is an integer of 1 2, 3, 4, or 5; preferably n is an integer of 1, or 2.

In certain embodiments, the RG, SP, HG, and PA are as provided herein.

### 7.2.6. Aspect 6

Described herein are compounds according to Formula (IIb):

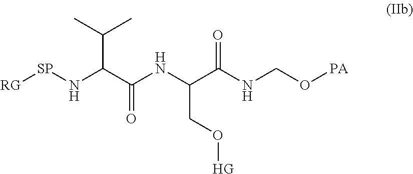

or a pharmaceutically acceptable salt, tautomer, solvate, stereoisomer, enantiomer, isotopologue, or prodrug thereof.

In certain embodiments, RG is

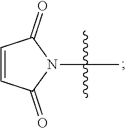 [SMILES: *N1C(=O)C=CC1=O]

SP is —(CH₂)ₙ—C(═O)—; and n is an integer of 1 2, 3, 4, or 5; preferably n is an integer of 1, or 2.

In certain embodiments, the RG, SP, HG, and PA are as provided herein.

### 7.2.7. Aspect 17

Described herein is a ligand-drug conjugate or a pharmaceutically acceptable salt or solvate thereof, wherein the ligand-drug conjugate comprises a structure of formula (III):

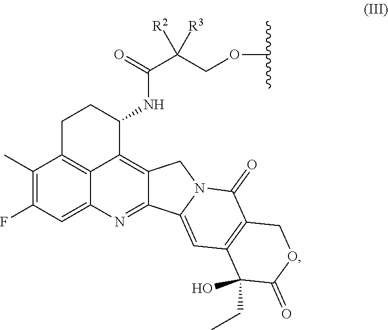

wherein
each of R² and R³ is, independently, hydrogen, halogen, or alkyl.

In some embodiments, the ligand-drug conjugate comprises a structure of formula (E2):

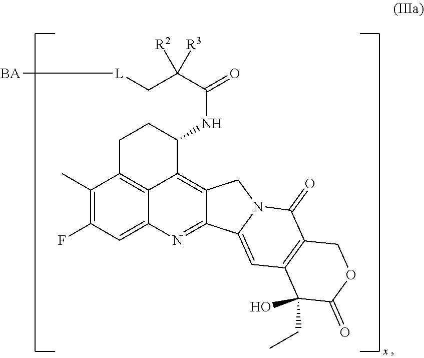

wherein
BA is a binding agent selected from a humanized, chimeric, or human antibody or an antigen binding fragment of an antibody;
L is a covalent linker as described here; and
x is as provided herein.

In some embodiments, the antibody is Ifinatamab, patritumab, cofetuzumab, or trastuzumab. In one embodiment, the antibody is Ifinatamab.

In one embodiment, R² and R³ are hydrogen.

In one embodiment, R² and R³ are methyl.

In one embodiment, R² is methyl and R³ is F.

In one embodiment, the carbon that R² and R³ connect to is in the S configuration.

In one embodiment, the carbon that R² and R³ connect to is in the R configuration.

In one embodiment, the covalent linker, L, has the structure of Formula (I-L):

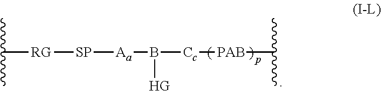

In one embodiment, the covalent linker, L, has the structure of Formula (Ia-L):

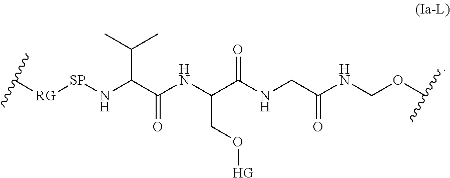

In one embodiment, the covalent linker, L, has the structure of Formula (Ib-L):

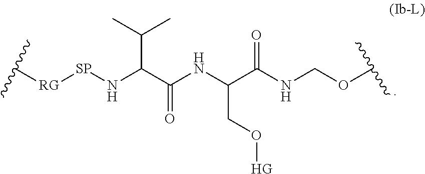

In certain embodiments, the RG, SP, HG, PAB, A, B, C, a, c, and p are as provided herein.

### 7.2.8. Aspect 18

Described herein is a compound having formula (IV):

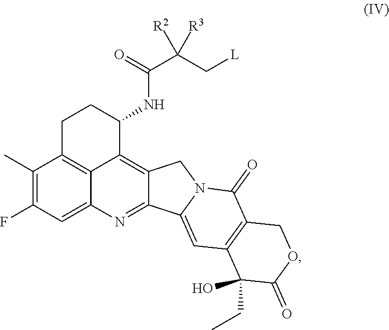

or a pharmaceutically acceptable solvate, stereoisomer, or derivative thereof, wherein
L is a covalent linker as described here;
each of R² and R³ is, independently, hydrogen, halogen, or alkyl.

In one embodiment, R² and R³ are hydrogen.

In one embodiment, R² and R³ are methyl.

In one embodiment, R² is methyl and R³ is F.

In one embodiment, the carbon that R² and R³ connect to is in the S configuration.

In one embodiment, the carbon that R² and R³ connect to is in the R configuration.

In one embodiment, the covalent linker, L, has the structure of Formula (II-L):

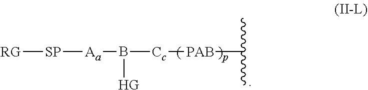

In one embodiment, the covalent linker, L, has the structure of Formula (IIa-L):

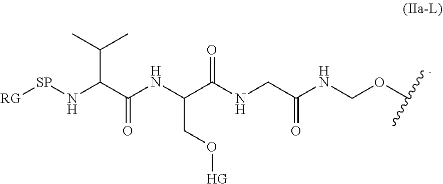

In one embodiment, the covalent linker, L, has the structure of Formula IIb-L):

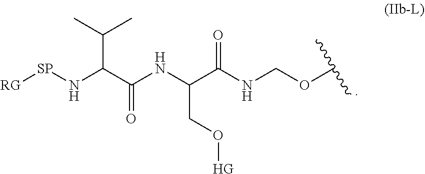

In certain embodiments, the RG, SP, HG, PAB, A, B, C, a, c, and p are as provided herein.

### 7.3. Payload

Provided herein is a compound, or a pharmaceutically acceptable solvate, stereoisomer, or derivative thereof, wherein the compound is a compound formula (V):

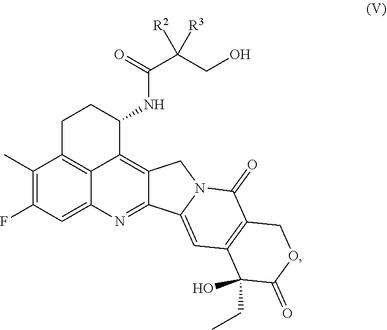

or a pharmaceutically acceptable solvate, stereoisomer, or derivative thereof, wherein
each of R² and R³ is, independently, hydrogen, halogen, or alkyl.

In one embodiment, R² and R³ are hydrogen.

In one embodiment, R² and R³ are methyl.

In one embodiment, R² is methyl, and R³ is F.

In one embodiment, the carbon that R² and R³ connect to is in the S configuration.

In one embodiment, the carbon that R² and R³ connect to is in the R configuration.

### 7.4. Methods or Processes of Making the Conjugates

In some embodiments, set forth herein is a method of preparing an antibody-drug conjugate comprising the step of contacting a binding agent with a linker-payload compound under conditions suitable for forming a bond between the binding agent and the linker-payload compound. Also provided is a method of preparing a compound of Formula (I), Formula (Ia), or Formula (Ib) under conditions suitable for forming a bond between the binding agent and the linker-payload compound.

In certain embodiments, the antibody is reacted or treated with a reactive linker-payload to form an antibody-payload conjugate. The reaction can proceed under conditions deemed suitable by those of skill in the art. In certain embodiments, the antibody is contacted with the reactive linker-payload compound under conditions suitable for forming a bond between the antibody and the linker-payload compound. Suitable reaction conditions are well known to those in the art.

Examples of such reactions are provided in the Examples below.

In some embodiments, set forth herein is a method of making a conjugate comprising treating or contacting a compound with a binding agent under coupling conditions, wherein the compound comprises a reactive linker bonded to at least one payload moiety and, wherein the compound which reacts with a binding agent is a compound of Formula (II), Formula (IIa), or Formula (IIb) or a pharmaceutically acceptable salt, solvate, stereoisomer, or derivative thereof.

### 7.5. Pharmaceutical Compositions

Provided herein is a pharmaceutical composition a compound set forth herein, or pharmaceutically acceptable salt thereof, and a pharmaceutically acceptable excipient.

### 7.6. Methods of Using

In some embodiments, set forth herein is a method of treating a disease or disorder in a patient in need thereof comprising administering to the patient a compound or pharmaceutical composition set forth herein. In some embodiments, the administered compound is an antibody-drug conjugate set forth herein.

In some embodiments, set forth herein is a method of treating or preventing a disease, disorder, or condition selected from the group consisting of a proliferative disorder, a neurodegenerative disorder, an immunological disorder, an autoimmune disease, an inflammatory disorder, a dermatological disease, a metabolic disease, cardiovascular disease, and a gastrointestinal disease comprising administering to the subject of an effective treatment amount of a compound or pharmaceutical composition set forth herein. In some embodiments, the administered compound is an antibody-drug conjugate set forth herein.

In some embodiments, set forth herein is a method of treating a proliferative disease, a metabolic disease, inflammation, or a neurodegenerative disease in a subject comprising administering to the subject of an effective treatment amount of a compound or pharmaceutical composition set forth herein. In some embodiments, set forth herein is a method of treating a proliferative disease in a subject comprising administering to the subject of an effective treatment amount of a compound or pharmaceutical composition set forth herein. In some embodiments, the administered compound is an antibody-drug conjugate set forth herein.

In some embodiments, set forth herein is a method of treating a metabolic disease in a subject comprising administering to the subject of an effective treatment amount of a compound or pharmaceutical composition set forth herein. In some embodiments, the administered compound is an antibody-drug conjugate set forth herein.

In some embodiments, set forth herein is a method of treating inflammation in a subject comprising administering to the subject of an effective treatment amount of a compound or pharmaceutical composition of set forth herein. In some embodiments, the administered compound is an antibody-drug conjugate set forth herein.

In some embodiments, set forth herein is a method of treating a neurodegenerative disease in a subject comprising administering to the subject of an effective treatment amount of a compound or pharmaceutical composition set forth herein. In some embodiments, the administered compound is an antibody-drug conjugate set forth herein.

Although any methods and materials similar or equivalent to those described herein can be used in the practice or testing of the present invention, the preferred methods and materials are now described. All patents, applications and non-patent publications mentioned in this specification are incorporated herein by reference in their entireties.

### 8. EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v), and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet (s), doublet (d), triplet (t), quartet (q), quintet (p), sextet (h), septet (hept), multiplet (m), or a combination thereof; br stands for broad. LC/MS data was recorded by using Agilent 1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 18.0.

For the sake of conciseness, certain abbreviations are used herein. One example is the single letter abbreviation to represent amino acid residues. The amino acids and their corresponding three letter and single letter abbreviations are as follows:

### List of Abbreviations for amino acids

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

In the following examples, the following abbreviations are used:

### List of Abbreviations

| | |
|---|---|
| TEA | triethylamin |
| THF | tetrahydrofuran |
| MeOH | Methanol |
| TEMPO | 2,2,6,6-Tetramethylpiperidine 1-oxyl |
| PhI(OAc)₂ | Diacetoxyiodo)benzene |
| PyBOP | Benzotriazol-1-yl-oxytripyrrolidino- |
| | phosphonium Hexafluorophosphate |
| HOBt | 1-Hydroxybenzotriazole |
| DIPEA | N,N-Diisopropylethylamine |
| DMF | N,N-Dimethylformamide |
| NH4Cl | Ammonium chloride |
| LiOH | Lithium hydroxide |
| HCl | Hydrochloric acid |
| PNP | Bis(4-nitrophenyl)carbonate |
| TFA | Trifluoroacetic acid |
| K₂CO₃ | Potassium carbonate |
| MeI | Methyl iodide |
| Na₂SO₄ | Sodium sulfate |
| EtOAc | Ethyl acetate |
| MeONa | Sodium methoxide |
| prep-HPLC | Preparative high performance liquid |
| | chromatography |
| TBSCl | t-Butyldimethylchlorosilane |
| Cu(OAc)₂ | Anhydrous Cupric Acetate |
| Pb(OAc)₄ | lead tetraacetate |
| TSTU | 2-Succinimido-1,1,3,3-tetra-methyluronium |
| | tetrafluor |
| Et₂N | Diethylamine |
| r.t. | room temperature |
| MS | mass spectrometry |
| ESI | electron spray ionization |
| FA | formic acid |
| NHS | N-hydroxysuccinimide |
| MTBE | methyl tert-butyl ether |
| NHS | N-hydroxysuccinimide |
| Fmoc | 9-fluorenylmethoxycarbonyl protecting group |
| DBU | 1,8-diazabicyclo[5.4.0]undec-7-ene |
| DCC | dicyclohexylcarbodiimide |
| HPLC | high pressure liquid chromatography |
| Mc-OSu | maleimidocaproyl N-hydroxysuccimidyl ester |
| DMSO | dimethylsulfoxide;. |
| MMAE | mono-methyl auristatin E or N-methylvaline- |
| | valine-dolaisoleuine-dolaproine-phenylalanine |
| AEVB | auristatin E valeryl benzylhydrazone, acid |
| | labile linker through the C-terminus of AE |

### Example 1

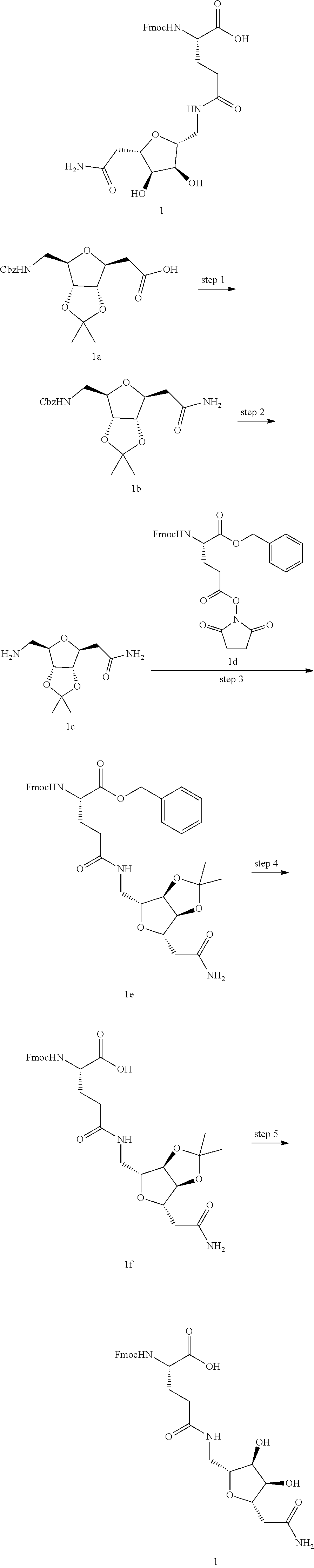

### Step 1

### Benzyl (((3aR,4R,6S,6aS)-6-(2-amino-2-oxoethyl)-2,2-dimethyltetrahydrofuro[3,4-d][1,3]dioxol-4-yl)methyl)carbamate (1b)

To a suspension of 1a (2.01 g, 5.47 mmol) in DMF (20 mL) were added PyBOP (3.42 g, 6.57 mmol), HOBt (887 mg, 6.57 mmol), DIPEA (2.12 g, 16.42 mmol) and NH₄Cl (2.93 g, 54.7 mmol). The resulting brown suspension was stirred at 50° C. overnight. After complete reaction, the mixture was quenched with water (20 mL), extracted with EtOAc (30 mL*3). The combined organic layers were washed with brine (30 mL*3), dried over anhydrous Na₂SO₄, filtered and concentrated under vacuum to give a residue which was purified by silica gel column chromatography (DCM/MeOH=100/0 to 90/10) to afford 20a (1.81 g, 90.6% yield) as a pale-yellow solid.

MS (ESI) m/z: 365.4 [M+H]⁺

### Step 2

### 2-((3aS,4S,6R,6aR)-6-(aminomethyl)-2,2-dimethyltetrahydrofuro[3,4-d][1,3]dioxol-4-yl)acetamide (1c)

1c (1.80 g, 4.94 mmol) was dissolved in a mixed solvent of THF (15 mL) and MeOH (15 mL) followed by the addition of Pd/C (wet base, 10%, 450 mg). The resulting mixture was stirred at 25° C. for 3 h. After complete reaction, the reaction mixture was filtered and the filtrate was concentrated under reduced pressure to afford 1c as a grey syrup (1.14 g, crude). The crude product was used directly in next step without purification.

MS (ESI) m/z: 231.4 [M+H]⁺

### Step 3

### Benzyl N2-(((9H-fluoren-9-yl)methoxy)carbonyl)-N5-(((3aR,4R,6S,6aS)-6-(2-amino-2-oxoethyl)-2,2-dimethyltetrahydrofuro[3,4-d][1,3]dioxol-4-yl)methyl)-L-glutaminate (1e)

To a mixture of 1c (900 mg, crude) and id (2.18 g, 3.91 mmol) (commercially available in THF (20 mL) was added saturated aqueous NaHCO₃ (5 mL). The mixture was stirred at r.t. for 1 h. After the reaction was complete, the mixture was diluted with water (20 mL), extracted with EtOAc (20 mL*2). The combined organic layers were washed with brine (30 mL), dried over Na₂SO₄, filtered and the filtrate was concentrated under vacuum to provide a brown oil which was purified by flash column (DCM/MeOH=100/0 to 85/15) to afford 1e as a white solid (2.15 g, 82.1% yield).

MS (ESI) m/z: 672.6 [M+H]⁺

### Step 4

### N2-(((9H-fluoren-9-yl)methoxy)carbonyl)-N5-(((3aR,4R,6S,6aS)-6-(2-amino-2-oxoethyl)-2,2-dimethyltetrahydrofuro[3,4-d][1,3]dioxol-4-yl)methyl)-L-glutamine (1f)

1e (2.10 g, 3.13 mmol) was dissolved in a mixed solvent of THF (20 mL) and MeOH (20 mL) followed by the addition of Pd/C (wet base, 10%, 525 mg). The resulting mixture was stirred at 25° C. for 3 h. After complete reaction, the reaction mixture was filtered and the filtrate was concentrated under reduced pressure to afford 1f (1.87 g, crude) as a white solid which was used directly in next step without purification.

MS (ESI) m/z: 582.5 [M+H]⁺

### Step 5

### N2-(((9H-fluoren-9-yl)methoxy)carbonyl)-N5-(((2R,3S,4R,5S)-5-(2-amino-2-oxoethyl)-3,4-dihydroxytetrahydrofuran-2-yl)methyl)-L-glutamine (1)

1f (1.80 g, 3.09 mmol) was dissolved in DCM (20 mL) and the solution was cooled to 0° C. before addition of a mixed solvent of TFA/H₂O (20 mL, v/v=9:1) dropwise. The resulting mixture was further stirred at 0° C. for 1 h. After complete reaction, the reaction mixture was purified by prep-HPLC (Method: column: XBridge Prep C18 OBD 5 μm 19*250 mm; Mobile phase: A—water (0.05% trifluoroacetic acid): B—acetonitrile; Flow rate: 20 mL/min)) to afford 1 as a white solid (630 mg, 37.6%).

MS (ESI) m/z: 542.5 [M+H]⁺

### Example 2

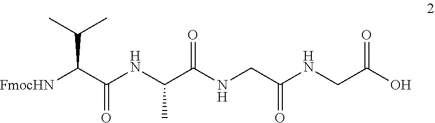

2 was synthesized according to a standard protocol of solid-phase peptide synthesis (Muriel et al., Methods and protocols of modern solid phase peptide synthesis, Mol. Biotechnol., 2006, 33, 239-254) using readily commercially available starting materials.

### Example 3

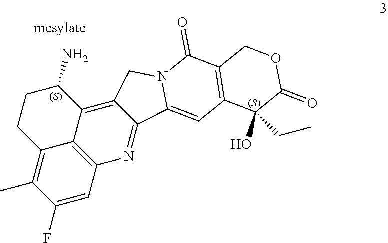

3 was purchased from ShangHai HaoYuan MedChemExpress CO. LTD.

### Example 4

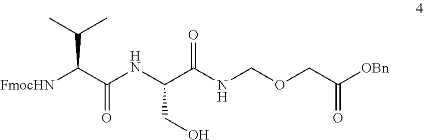

4 was synthesized according to reported procedures (Wei et al., Synthesis and Evaluation of Camptothecin Antibody Drug Conjugates, ACS Med. Chem. Lett. 2019, 10, 1386-1392; U.S. Pat. No. 9,808,537 B2) using readily commercially available starting materials.

### Example 5

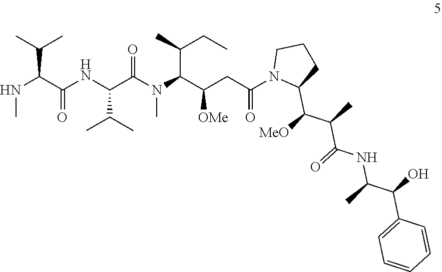 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

5 was purchased from ShangHai HaoYuan MedChemExpress CO. LTD.

The other readily commercially available reactants or reagents are not specifically listed or numbered.

### Example 6

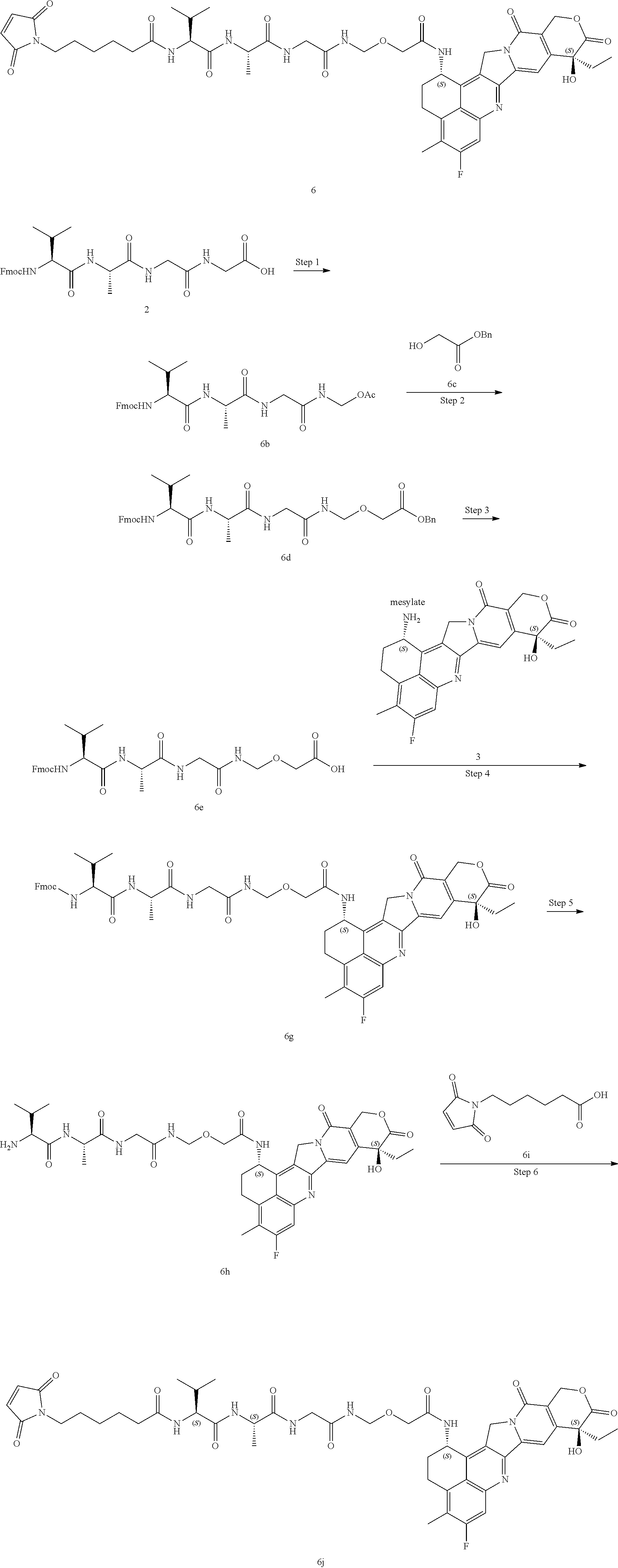

### Step 1

### (((9H-fluoren-9-yl)methoxy)carbonyl)-L-valyl-L-alanylglycylglycine (6b)

To a mixture of 2 (1.60 g, 3.05 mmol), Cu(OAc)₂ (221 mg, 1.22 mmol) in DMF (20 mL) were added Pb(OAc)₄ (1.62 g, 3.66 mmol) and HOAc (348 μL, 6.10 mmol). The resulted black mixture was purged with N balloon for 3 times then stirred at 70° C. for 1 h, the black mixture turns into a dark blue mixture. On completion. The mixture was diluted with EtOAc (100 mL) then washed with brine, dried over Na₂SO₄, filtered, and concentrated under vacuum to give 6b (1.40 g, 85.2% yield) as a white solid.

MS (ESI) m/z: 561.5 [M+Na]⁺

### Step 2

### benzyl (5S,8S)-1-(9H-fluoren-9-yl)-5-isopropyl-8-methyl-3,6,9,12-tetraoxo-2,15-dioxa-4, 7,10,13-tetraazaheptadecan-17-oate (6d)

To a solution of 6b (0.60 g, 1.12 mmol) and 4 Å molecular sieve in THF (6 mL) were added 6c (0.56 g, 3.34 mmol) and Sc(OTf)₃ (0.55 g, 1.12 mmol). The resulted yellow suspension was stirred at r.t. for overnight. On completion of the reaction, the reaction mixture was filtered and the cake was washed with THF. Combined THF phases were then quenched by addition of sat. aqueous NaHCO₃ (50 mL) and extracted with EtOAc (100 mL). After separation the combined organic layers were washed with brine (50 mL), dried over Na₂SO₄, filtered and the filtrate was concentrated under vacuum to give a residue which was purified by silica column gel chromatography (eluent: DCM/MeOH=100/0 to 85/15) to afford 6d (600 mg, 83.5% yield) as yellow solid.

MS (ESI) m/z: 667.5 [M+Na]⁺

### Step 3

### (5S,8S)-1-(9H-fluoren-9-yl)-5-isopropyl-8-methyl-3,6,9,12-tetraoxo-2,15-dioxa-4,7,10,13-tetraazaheptadecan-17-oic acid (6e)

To a solution of 6d (215 mg, 0.33 mmol) in MeOH was added wet Pd/C (20 mg). Then the mixture was purged with H₂ balloon for 3 times and stirred at r.t. for 2 h. On completion of the reaction. The mixture was filtered through syringe filter head and the filtrate was concentrated under vacuum to give 6e (185 mg, crude) as a white solid.

MS (ESI) m/z: 578.4 [M+Na]⁺

### Step 4

### (9H-fluoren-9-yl)methyl ((10S,13S)-1-(((1S,9S)-9-ethyl-5-fluoro-9-hydroxy-4-methyl-10, 13-dioxo-2,3,9,10,13,15-hexahydro-1H,12H-benzo[de]pyrano[3′,4′:6,7]indolizino[1,2-b]quinolin-1-yl)amino)-10,14-dimethyl-1,6,9,12-tetraoxo-3-oxa-5,8,11-triazapentadecan-13-yl)carbamate (6g)

To a solution of 6e (185 mg, 0.33 mmol) in DMF (2 mL) were added HBTU (190 mg, 0.5 mmol) and DIPEA (170 μL, 129 mg, 1 mmol). The resulted yellow solution was stirred at r.t. for 5 min then 3 (195 mg, 0.37 mmol) was added. The mixture was stirred at r.t. for 60 min. On completion of the reaction. The mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 6g (150 mg, 46.3% yield) as a white powder.

MS (ESI) m/z: 972.5 [M+H]⁺

### Step 5

### (S)-2-amino-N—((S)-1-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-1-oxopropan-2-yl)-3-methylbutanamide (6h)

To a solution of 6g (30 mg, 0.03 mmol) in DMF (2 mL) was added Et₂N (30 μL, 22.59 mg, 0.31 mmol). The mixture was stirred at r.t. for 1 h. On completion of the reaction. The mixture was concentrated under vacuum to give 6h (24 mg, crude) as a yellow oil.

### Step 6

### 6-(2,5-dioxo-2,5-dihydro-1H-pyrrol-1-yl)-N-((10S,13S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10,14-dimethyl-1,6,9,12-tetraoxo-3-oxa-5,8,11-triazapentadecan-13-yl)hexanamide (6)

To a solution of 6i (13 mg) in DMF (2 mL) were added HBTU (23 mg, 0.06 mmol) and DIPEA (16 μL, 12 mg, 0.09 mmol). The resulted yellow solution was stirred at r.t. for 5 min then 6h (24 mg) was added. The mixture was stirred at r.t. for 60 min. On completion of the reaction. The mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 6 (18 mg, 61.5% yield) as a white powder.

MS (ESI) m/z: 943.6 [M+H]⁺

### Example 7

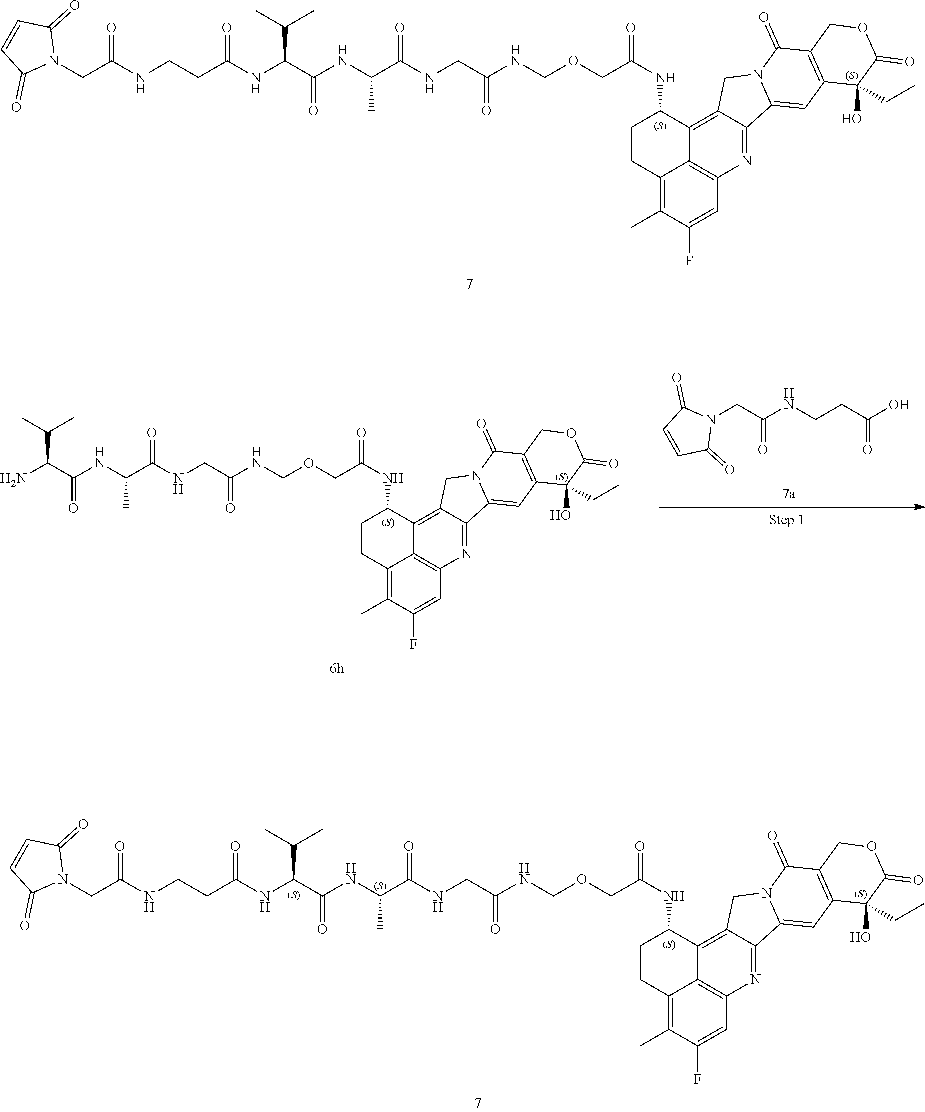

### Step 1

### (S)-2-(3-(2-(2,5-dioxo-2,5-dihydro-1H-pyrrol-1-yl)acetamido)propanamido)-N—((S)-1-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-1-oxopropan-2-yl)-3-methylbutanamide (7)

To a solution of 7a (19 mg) in DMF (2 mL) were added HBTU (31 mg, 0.08 mmol) and DIPEA (22 μL, 16 mg, 0.12 mmol). The resulted yellow solution was stirred at r.t. for 5 min then 6h (30 mg) was added. The mixture was stirred at r.t. for 60 min. On completion of the reaction. The mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 7 (20 mg, 50.7% yield) as a white powder.

MS (ESI) m/z: 958.5 [M+H]⁺

### Example 8

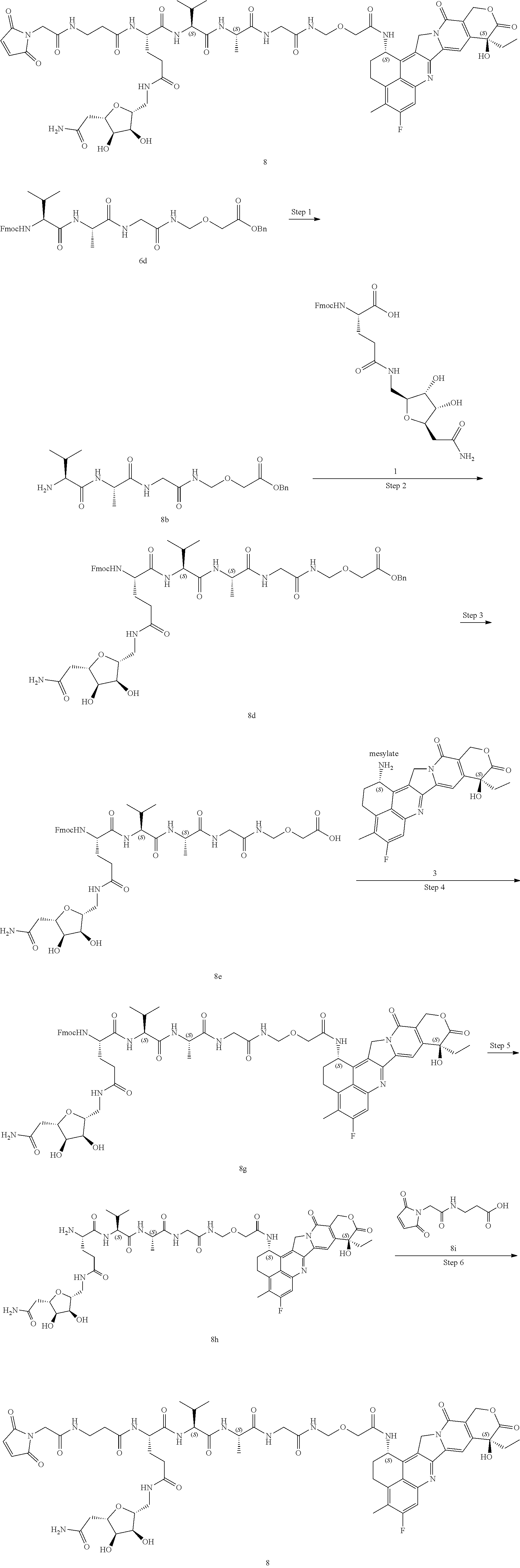

### Step 1

### benzyl (10S,13S)-13-amino-10,14-dimethyl-6,9,12-trioxo-3-oxa-5,8,11-triazapentade-canoate (8b)

To a solution of 6d (600 mg, 0.93 mmol) in DMF (5 mL) was added Et₂NH (948 μL, 681 mg, 9.31 mmol). The mixture was stirred at r.t. for 1 h. On completion of the reaction. The mixture was concentrated under vacuum to give 8b (395 mg, crude) as a yellow solid.

### Step 2

### benzyl (5S,8S,11S)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-8-isopropyl-11-methyl-3,6,9,12,15-pentaoxo-2,18-dioxa-4,7,10,13,16-pentaazaicosan-20-oate (8d)

To a solution of 1 (604 mg, 1.12 mmol) in THF (5 mL) were added EDCI (214 mg, 1.12 mmol), HOBt (151 mg, 1.12 mmol) and DIPEA (238 μL, 180 mg, 1.4 mmol). The resulted yellow solution was stirred at r.t. for 15 min then 8b (395 mg, 0.93 mmol) was added. The mixture was stirred at r.t. for 16 h. On completion of the reaction. The mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 8d (350 mg, 40.8% yield) as a white powder.

MS (ESI) m/z: 968.7 [M+Na]⁺

### Step 3

### (5S,8S,11S)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-8-isopropyl-11-methyl-3,6,9,12,15-pentaoxo-2,18-dioxa-4,7,10,13,16-pentaazaicosan-20-oic acid (8e)

To a solution of 8d (190 mg, 0.33 mmol) in MeOH/THF was added wet Pd/C (20 mg) then purge with H₂ balloon for 3 times and stirred at r.t. for 3 h. On completion of the reaction. The mixture was filtered Pd/C through syringe filter head and the filtrate was concentrated under vacuum to give 8e (172 mg, crude) as a white solid.

### Step 4

### (9H-fluoren-9-yl) methyl ((6S,9S,12S)-1-((2R,3S,4R,5S)-5-(2-amino-2-oxoethyl)-3,4-dihydroxytetrahydrofuran-2-yl)-21-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-9-isopropyl-12-methyl-3,7,10,13,16,21-hexaoxo-19-oxa-2,8,11,14,17-pentaazahenicosan-6-yl)carbamate (8g)

To a solution of 8e (172 mg, 0.33 mmol) in DMF (2 mL) were added HBTU (109.75 mg, 0.29 mmol) and DIPEA (68.5 uL, 51.95 mg, 0.4 mmol). The resulted yellow solution was stirred at r.t. for 5 min then 3 (128 mg, 0.24 mmol) was added. The mixture was stirred at r.t. for 60 min. On completion of the reaction. The mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 8g (100 mg, 39.1% yield) as a white powder.

MS (ESI) m/z: 1272.51 [M+Na]⁺

### Step 5

### (S)-2-amino-N5-(((2R,3S,4R,5S)-5-(2-amino-2-oxoethyl)-3,4-dihydroxytetrahydrofuran-2-yl)methyl)-N1-((10S,13S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10,14-dimethyl-1,6,9,12-tetraoxo-3-oxa-5,8,11-triazapentadecan-13-yl)pentanediamide (8h)

To a solution of 8g (30 mg, 0.02 mmol) in DMF (2 mL) was added Et₂N (24 μL, 17 mg, 0.24 mmol). The mixture was stirred at r.t. for 1 h. On completion of the reaction. The mixture was concentrated under vacuum to give 8h (25 mg, crude) as a yellow oil.

### Step 6

### (S)—N5-(((2R,3S,4R,5S)-5-(2-amino-2-oxoethyl)-3,4-dihydroxytetrahydrofuran-2-yl)methyl)-2-(3-(2-(2,5-dioxo-2,5-dihydro-1H-pyrrol-1-yl)acetamido)propanamido)-N1-((10S,13S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10,14-dimethyl-1,6,9,12-tetraoxo-3-oxa-5,8,11-triazapentadecan-13-yl)pentanediamide (8)

To a solution of 8i (11 mg) in DMF (2 mL) were added HBTU (18 mg, 0.05 mmol) and DIPEA (12 μL, 9.1 mg, 0.07 mmol). The resulted yellow solution was stirred at r.t. for 5 min then 8h (25 mg) was added. The mixture was stirred at r.t. for 60 min. On completion of the reaction. The mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 8 (8.8 mg, 29.7% yield) as a white powder.

MS (ESI) m/z: 1281.8 [M+Na]⁺

### Example 9

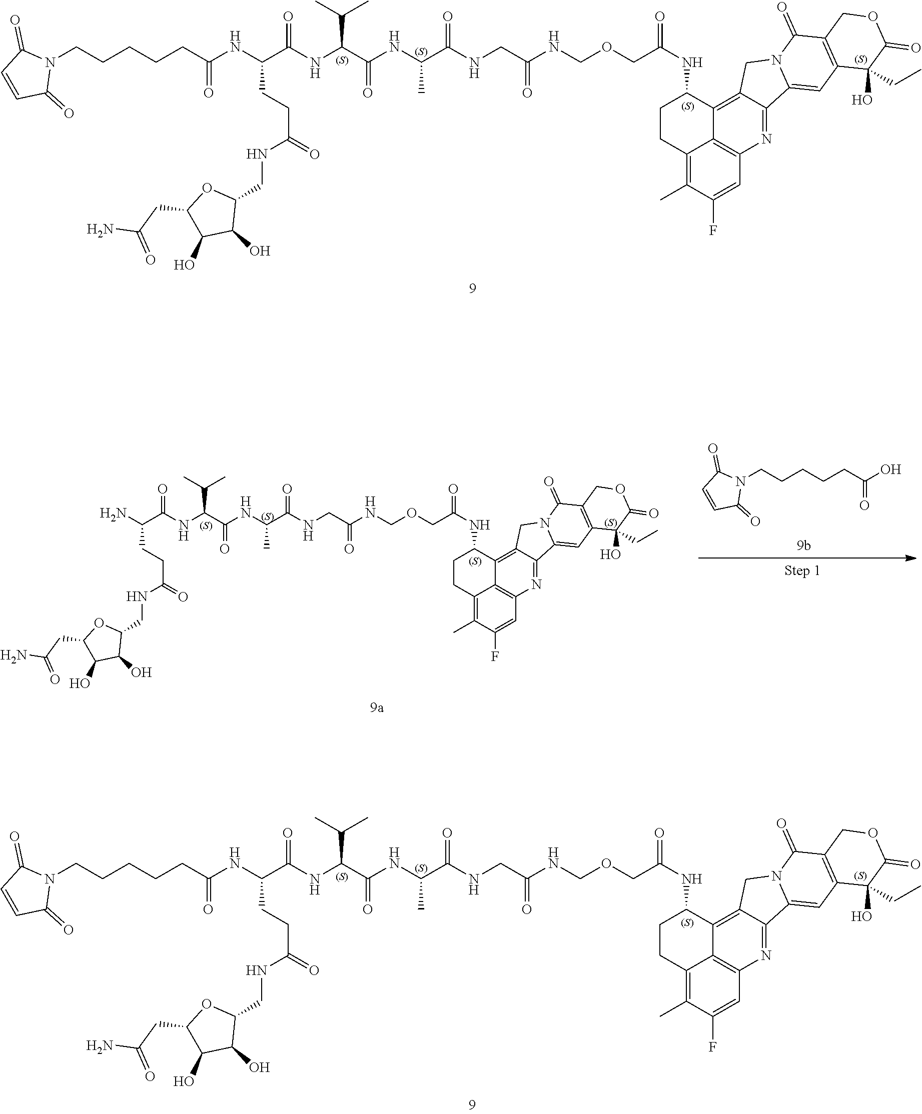

### Step 1

### (S)—N5-(((2R,3S,4R,5S)-5-(2-amino-2-oxoethyl)-3,4-dihydroxytetrahydrofuran-2-yl)methyl)-2-(6-(2,5-dioxo-2,5-dihydro-1H-pyrrol-1-yl)hexanamido)-N1-((10S,13S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10,14-dimethyl-1,6,9,12-tetraoxo-3-oxa-5,8,11-triazapentadecan-13-yl)pentanediamide (9)

To a solution of 9b (13 mg) in DMF (2 mL) were added HBTU (24 mg, 0.06 mmol) and DIPEA (16 μL, 12 mg, 0.09 mmol). The resulted yellow solution was stirred at r.t. for 5 min then 9a (40 mg) was added. The mixture was stirred at r.t. for 60 min. On completion of the reaction. The mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 9 (18 mg, 45.8% yield) as a white powder.

MS (ESI) m/z: 1266.8 [M+Na]⁺

### Example 10

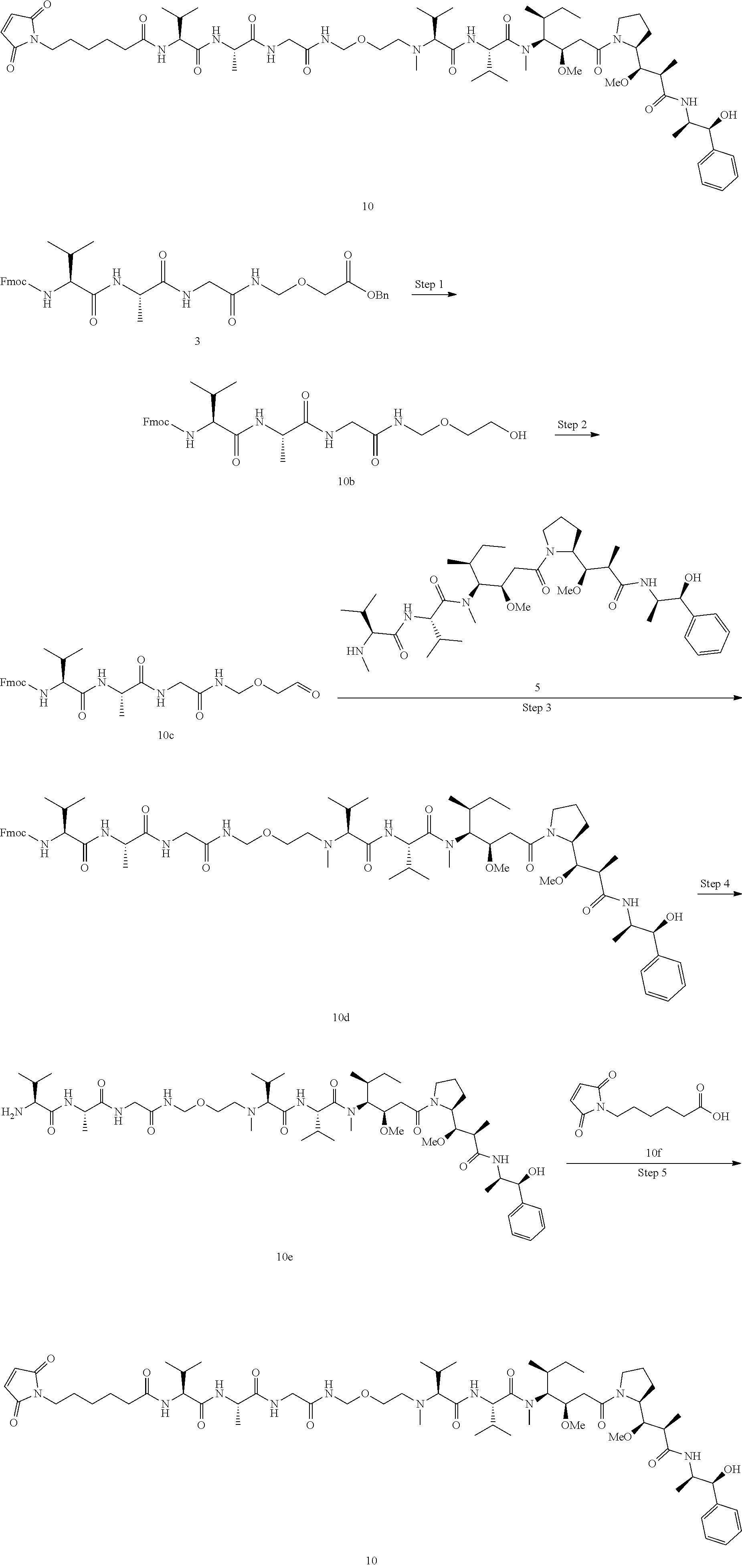

### Step 1

### (9H-fluoren-9-yl)methyl((10S,13S)-1-hydroxy-10,14-dimethyl-6,9,12-trioxo-3-oxa-5,8,11-triazapentadecan-13-yl)carbamate (10b)

To a solution of 3 (160 mg, 0.25 mmol) in anhydrous THF (2 mL) was added LiAlH₄ (1M in THF, 500 μL, 0.5 mmol) at −78° C. The reaction mixture was stirred at −78° C. for 1 h, then quenched with H₂O (100 μL) and passed through celite. The filtrate was concentrated under vacuum. The crude product was purified by silica column gel chromatography (eluent: DCM/MeOH=100/0 to 90/10) to afford 10b as white solid (120 mg, 89.6% yield).

MS (ESI) m/z: 563.4 [M+Na]⁺

### Step 2

### (9H-fluoren-9-yl)methyl((10S,13S)-10,14-dimethyl-1,6,9,12-tetraoxo-3-oxa-5,8,11-triazapentadecan-13-yl)carbamate (10c)

To a solution of 10b (120 mg, 0.22 mmol) in anhydrous DMSO (157 μL, 172 mg, 2.2 mmol) was added oxalyl chloride (95 μL, 140 mg, 1.1 mmol) at −78° C. The reaction mixture was stirred at −78° C. for 30 min, followed by the addition of Et₃N (457 μL, 334 mg, 3.3 mmol). The mixture was stirred at −78° C. for 30 min, then warm to r.t. and diluted with DCM (10 mL). The organic solution was washed subsequently with sat. NaHCO₃ (1 mL), H₂O (1 mL) and brine (1 mL). The organic phase was collected and dried over Na₂SO₄, filtered and the filtrate was concentrated under vacuum to give 10c as a pale-yellow oil. The crude product was used in the next step without further purification.

MS (ESI) m/z: 561.4 [M+Na]⁺

### Step 3

### (9H-fluoren-9-yl)methyl((3R,4S,7S,10S,21S,24S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11,21,25-tetramethyl-6,9,17,20,23-pentaoxo-2,14-dioxa-5,8,11,16,19,22-hexaazahexacosan-24-yl)carbamate (10d)

To a solution of 10c (20 mg, 0.037 mmol) in anhydrous MeOH (1 mL) was added 5 (26.56 mg, 0.037 mmol). The reaction mixture was stirred at r.t. for 15 min, followed by the addition of NaBH₃CN (12 mg, 0.19 mmol). The mixture was stirred at r.t. for 12 h. On completion of the reaction, the mixture was quenched with H₂O and then purified by prep-HPLC (TFA) (Method: column: XBridge Prep C18 OBD 5 μm 19*250 mm; Mobile phase: A—water (0.05% trifluoroacetic acid): B—acetonitrile; Flow rate: 20 mL/min, the fraction was lyophilized to give 10d (11 mg, 24.5% yield) as white powder.

MS (ESI) m/z: 1242.1 [M+H]⁺

### Step 4

### (S)-2-((2S,13S,16S)-16-amino-2-isopropyl-3,13,17-trimethyl-9,12,15-trioxo-6-oxa-3,8,11,14-tetraazaoctadecanamido)-N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethylbutanamide (10e)

To a solution of 10d (10 mg, 0.008 mmol) in DMF (0.5 mL) was added Et₂NH (8.4 μL, 5.8 mg, 0.08 mmol). The mixture was stirred at r.t. for 0.5 h. On completion of the reaction, the mixture was concentrated under vacuum to give 10e (10 mg) as a light-yellow solid, which was used directly in the next step without further purification.

MS (ESI) m/z: 1018.8 [M+H]⁺

### Step 5

### N-((3R,4S,7S,10S,21S,24S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11,21,25-tetramethyl-6,9,17,20,23-pentaoxo-2,14-dioxa-5,8,11,16,19,22-hexaazahexacosan-24-yl)-6-(2,5-dioxo-2,5-dihydro-1H-pyrrol-1-yl)hexanamide (10)

To a solution of 10e (8.15 mg, 0.008 mmol) in DMF (0.5 mL) were added HBTU (6.07 mg, 0.016 mmol) and DIPEA (4.19 μL, 3.11 mg, 0.024 mmol), then 10f (3.38 mg, 0.016 mmol) was added. The mixture was stirred at r.t. for 30 min. On completion of the reaction, the mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 10 (5 mg, 51.5% yield) as white powder.

MS (ESI) m/z: 1212.6 [M+H]⁺

### Example 11

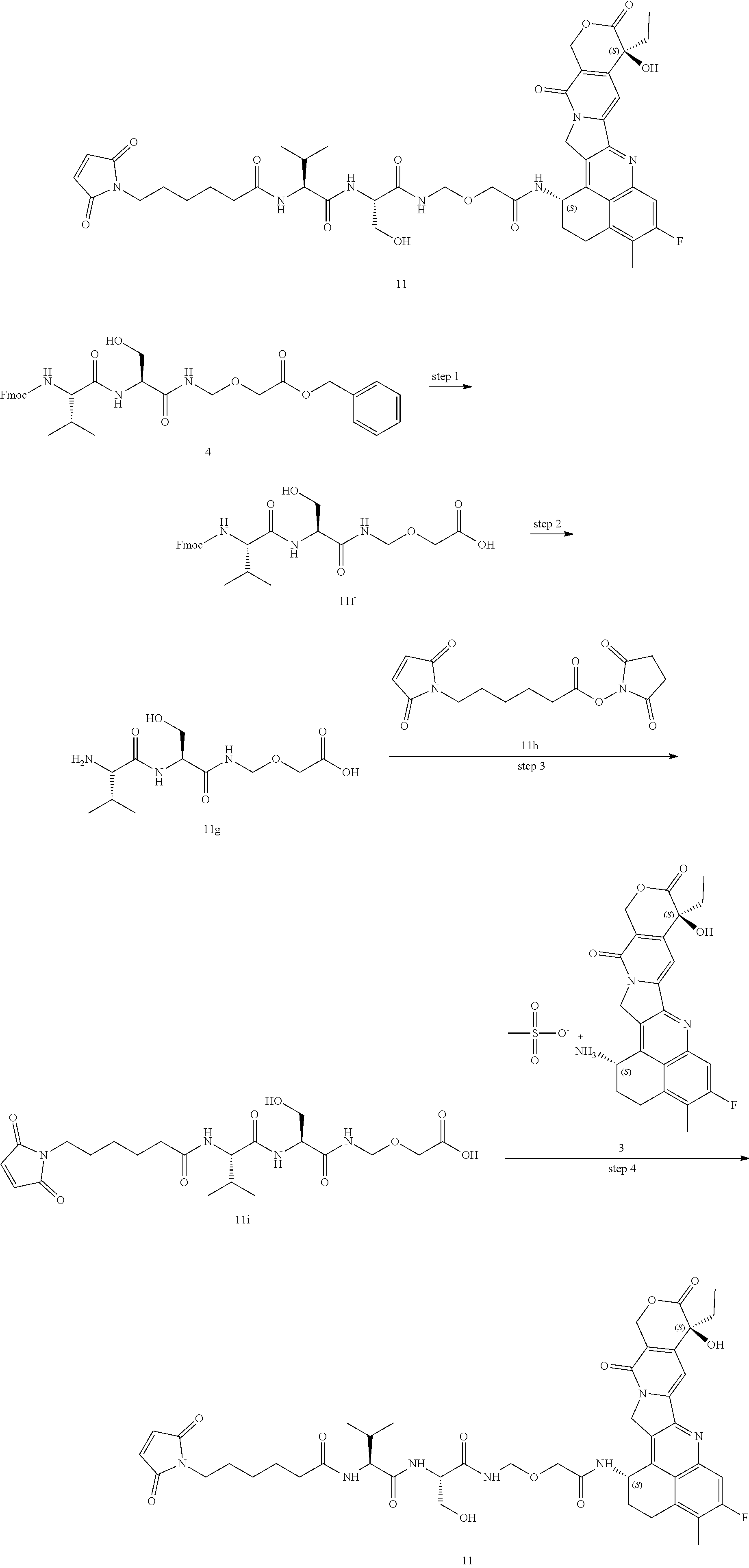

### Step 1

### (5R,8S)-1-(9H-fluoren-9-yl)-8-(hydroxymethyl)-5-isopropyl-3,6,9-trioxo-2,12-dioxa-4,7,10-triazatetradecan-14-oic acid (11f)

To a solution of 4 (100 mg, 0.17 mmol) in MeOH was added wet Pd/C (10 mg) then purge with H₂ balloon for 3 times and stirred at r.t. for 2 h. On completion of the reaction. The mixture was filtered Pd/C through syringe filter head and the filtrate was concentrated under vacuum to give 11f (90 mg, crude) as a white solid.

MS (ESI) m/z: 536.4 [M+Na]⁺

### Step 2

### 2-(((S)-2-((R)-2-amino-3-methylbutanamido)-3-hydroxypropanamido)methoxy)acetic acid (11g)

To a solution of 11f (90 mg, 0.175 mmol) in DMF (2 mL) was added Et₂N (180 uL, 128 mg, 1.75 mmol). The mixture was stirred at r.t. for 0.5 h. On completion of the reaction. The mixture was concentrated under vacuum to give 11 g (57 mg, crude) as a yellow solid.

MS (ESI) m/z: 292.4 [M+H]⁺

### Step 3

### (7S,10R)-17-(2,5-dioxo-2,5-dihydro-1H-pyrrol-1-yl)-7-(hydroxymethyl)-10-isopropyl-6,9,12-trioxo-3-oxa-5,8,11-triazaheptadecanoic acid (11i)

To a solution of 11g (29 mg, 0.098 mmol) and 11h (45 mg, 0.15 mmol) in DMF (2 mL) was added DIPEA (24 uL, 19 mg, 0.15 mmol). The mixture was stirred at r.t. for 1.5 h. On completion of the reaction. The mixture was purified by prep-HPLC (FA) (Method: column: XBridge Prep C18 OBD 5 um 19*250 mm; Mobile phase: A—water (0.1% formic acid): B—acetonitrile; Flow rate: 20 mL/min, the fraction was lyophilized to give 11i (18 mg, 25.3% yield) as a white powder.

MS (ESI) m/z: 507.4 [M+Na]⁺

### Step 4

### 6-(2,5-dioxo-2,5-dihydro-1H-pyrrol-1-yl)-N—((S)-1-(((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-3-hydroxy-1-oxopropan-2-yl)amino)-3-methyl-1-oxobutan-2-yl)hexanamide (11)

To a solution of 11i (18 mg, 0.037 mmol) in DMF (2 mL) were added HATU (15 mg, 0.039 mmol) and DIPEA (12 μL, 9.6 mg, 0.074 mmol). The resulted yellow solution was stirred at r.t. for 5 min then 3 (20 mg, 0.037 mmol) was added. The mixture was stirred at r.t. for 60 min. On completion of the reaction. The mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 11 (29 mg, 78.4% yield) as a white powder.

MS (ESI) m/z: 902.6 [M+H]⁺

### Example 12

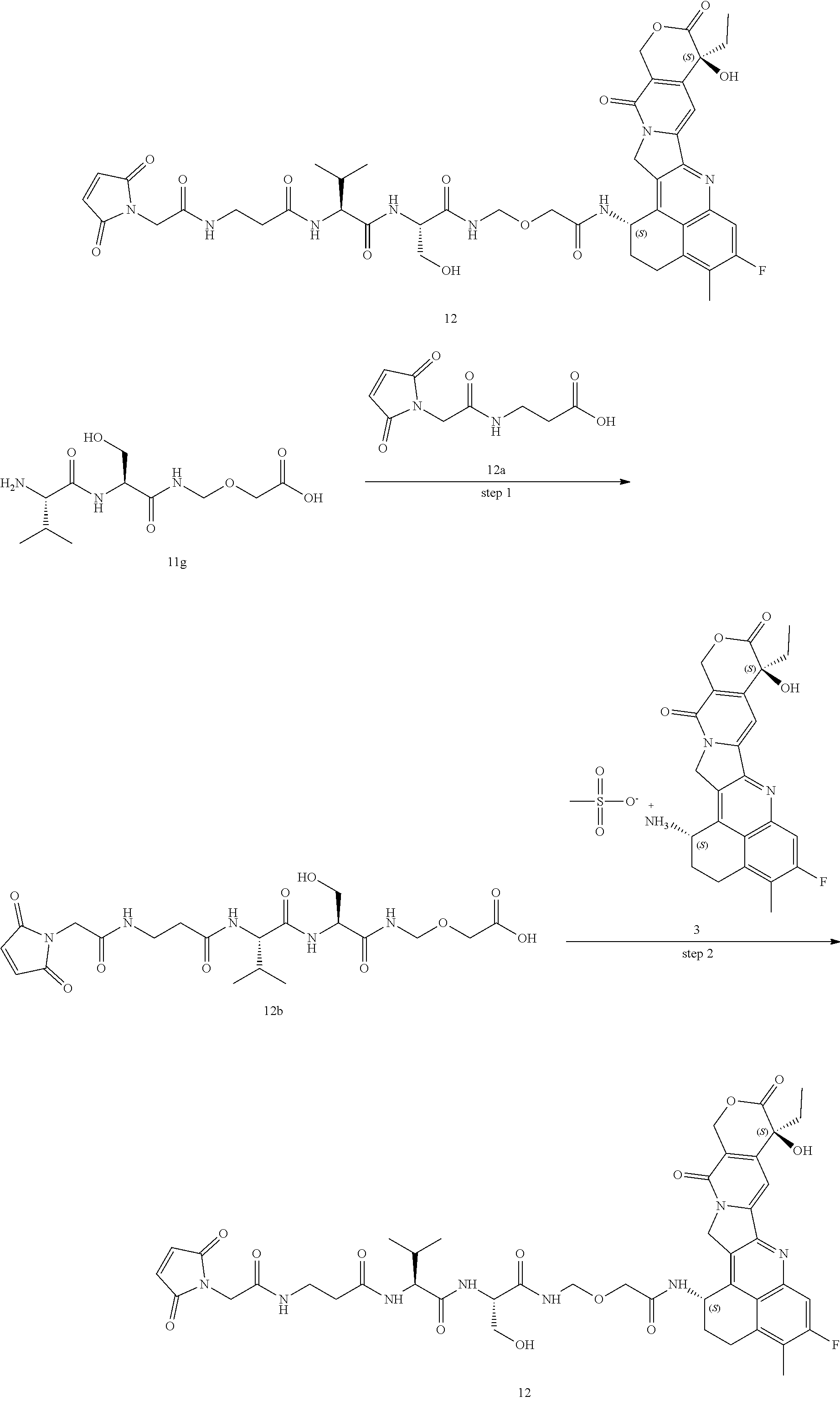

### Step 1

### (7S,10R)-17-(2,5-dioxo-2,5-dihydro-1H-pyrrol-1-yl)-7-(hydroxymethyl)-10-isopropyl-6,9,12,16-tetraoxo-3-oxa-5,8,11,15-tetraazaheptadecanoic acid (12b)

To a solution of 11g (29 mg, 0.098 mmol) and 12a (33 mg, 0.15 mmol) in DMF were added TSTU (41 mg, 0.14 mmol) and DIPEA (24 uL, 19 mg, 0.15 mmol). The mixture was stirred at RT for 1.5 hr. On completion of the reaction. The mixture was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 12b (23 mg, 31.400 yield) as a white powder.

MS (ESI) m/z: 522.4 [M+Na]⁺

### Step 2

### (S)-2-(3-(2-(2,5-dioxo-2,5-dihydro-1H-pyrrol-1-yl)acetamido)propanamido)-N—((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-3-hydroxy-1-oxopropan-2-yl)-3-methylbutanamide (12)

Compound 12 (13 mg, 32.9% yield) was synthesized according to synthetic procedure of step 6 of example 11.

MS (ESI) m/z: 917.6 [M+H]⁺

### Example 13

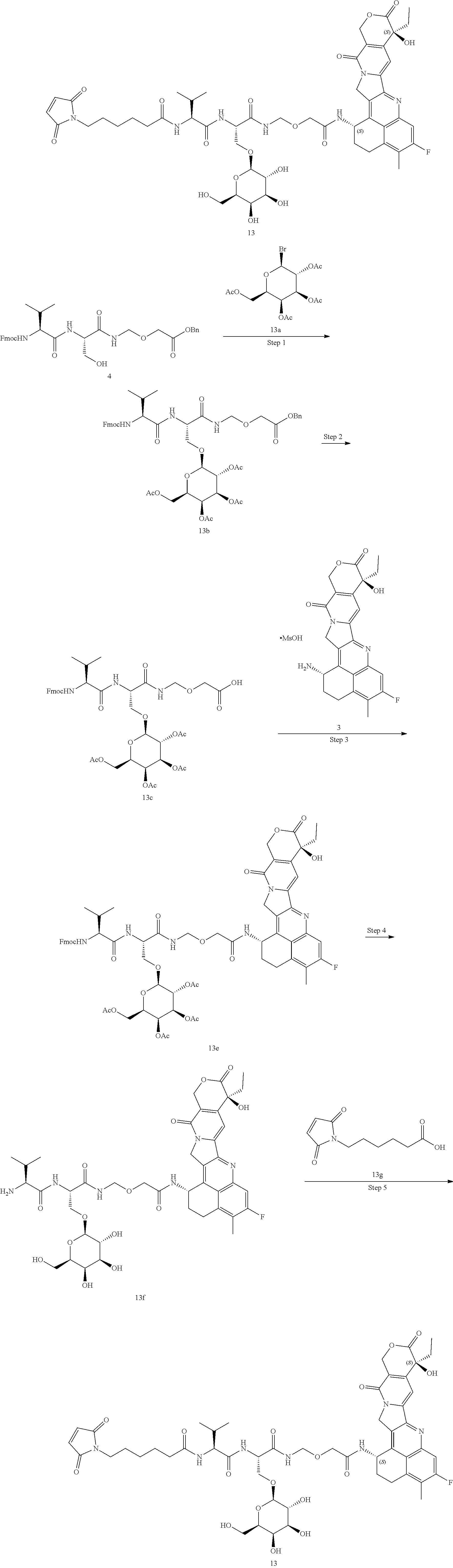

### Step 1

### (2R,3R,4S,5S,6R)-2-((S)-2-((S)-2-((((9H-fluoren-9-yl)methoxy)carbonyl)amino)-3-methylbutanamido)-3-(((2-(benzyloxy)-2-oxoethoxy)methyl)amino)-3-oxopropoxy)-6-(acetoxymethyl)tetrahydro-2H-pyran-3,4,5-triyl triacetate (13b)

To a solution of compound 4 (420 mg, 0.70 mmol) in DCM (12 mL) were added 13a (486 mg, 1.18 mmol) and 4 Å molecular sieves (800 mg). The mixture was stirred at r.t. for 30 min AgOTf (250 mg, 0.97 mmol) was added into the mixture and stirred at r.t. for 4 h. After complete reaction, the mixture was filtered and the filtrate was diluted by EA (200 mL), washed by saturated NaHCO₃ (50 mL*3) and brine (50 mL*3). The organic layer was dried over anhydrous Na₂SO₄, filtered and concentrated. The residue was purified by flash column chromatography (elute: PE/EA=100/0˜-70/30) to provide 13b (183 mg, 28.2% yield) as white solid.

MS (ESI) m/z: 956.5 [M+Na]⁺

### Step 2

### (5S,8S)-1-(9H-fluoren-9-yl)-5-isopropyl-3,6,9-trioxo-8-((((2R,3R,4S,5S,6R)-3,4,5-triacetoxy-6-(acetoxymethyl)tetrahydro-2H-pyran-2-yl)oxy)methyl)-2,12-dioxa-4,7,10-triazatetradecan-14-oic acid (13c)

To a solution of compound 13b (183 mg, 0.20 mmol) in MeOH (6 mL) was added Pd/C (10%, 20 mg). The mixture was stirred at H₂ atmosphere (15 psi) for 4 h. The mixture was filtered through a pad of celite, concentrated to give compound 13c (165 mg, crude) as white solid.

MS (ESI) m/z: 866.4 [M+Na]⁺

### Step 3

### (2R,3R,4S,5S,6R)-2-((S)-2-((S)-2-((((9H-fluoren-9-yl)methoxy)carbonyl)amino)-3-methylbutanamido)-3-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-3-oxopropoxy)-6-(acetoxymethyl)tetrahydro-2H-pyran-3,4,5-triyl triacetate (13e)

To a solution of compound 13c (165 mg, crude) in DMF (4 mL) were added 3 (109 mg, 0.21 mol), HATU (112 mg, 0.29 mmol) and DIEA (101 mg, 0.78 mmol). The mixture was stirred at r.t. for 30 min. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min to provide 13f (124 mg, 50.2% yield) as white solid.

MS (ESI) m/z: 1283.6 [M+Na]⁺

### Step 4

### (S)-2-amino-N—((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxo-3-(((2R,3R,4S,5R,6R)-3,4,5-trihydroxy-6-(hydroxymethyl)tetrahydro-2H-pyran-2-yl)oxy)propan-2-yl)-3-methylbutanamide (13f)

To a solution of compound 13e (124 mg, 0.098 mmol) in MeOH (6 mL) was added K₂CO₃ (136 mg, 0.98 mmol). The mixture was stirred at r.t. for 4 h. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min to provide 13f (42 mg, 49.1% yield) as white solid.

MS (ESI) m/z: 893.5 [M+Na]⁺

### Step 5

### 6-(2,5-dioxo-2,5-dihydro-1H-pyrrol-1-yl)-N—((S)-1-(((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxo-3-(((2R,3R,4S,5R,6R)-3,4,5-trihydroxy-6-(hydroxymethyl)tetrahydro-2H-pyran-2-yl)oxy)propan-2-yl)amino)-3-methyl-1-oxobutan-2-yl)hexanamide (13)

To a solution of compound 13g (11 mg, 0.048 mmol) in DMF (1.5 mL) were added HATU (17 mg, 0.043 mmol) and DIEA (6.2 mg, 0.048 mmol). The mixture was stirred at r.t. for 15 min. Compound 13f (21 mg, 0.024 mmol) was added into the mixture and was stirred at r.t. for 15 min. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min to provide 13 (6.3 mg, 24.6% yield) as white solid.

MS (ESI) m/z: 1086.6 [M+Na]⁺

### Example 14

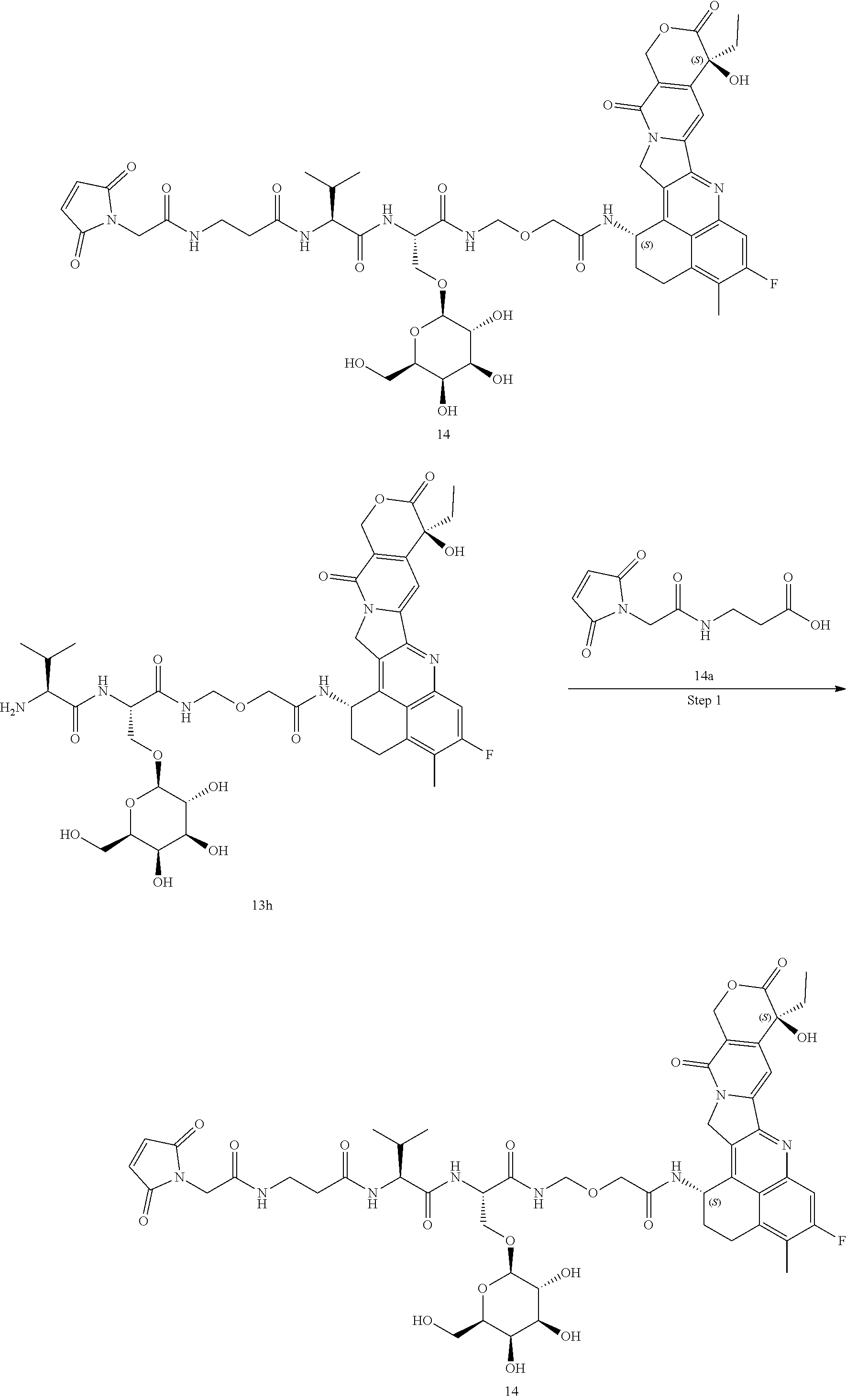

### Step 1

Compound 14 (3.6 mg, 13.8% yield) was synthesized according to synthetic procedure of step 5 of example 13.

MS (ESI) m/z: 1101.6 [M+Na]⁺

### Example 15

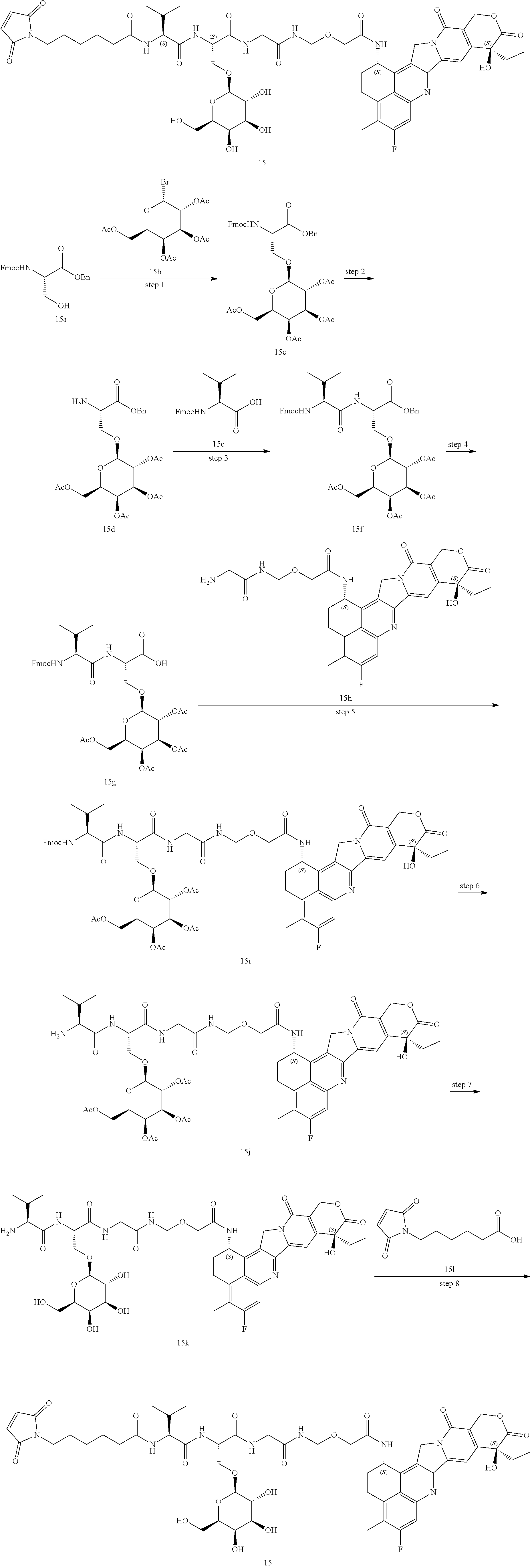

### Step 1

### (2R,3R,4S,5S,6R)-2-((S)-2-((((9H-fluoren-9-yl)methoxy)carbonyl)amino)-3-(benzyloxy)-3-oxopropoxy)-6-(acetoxymethyl)tetrahydro-2H-pyran-3,4,5-triyl triacetate (15c)

To a solution of compound 15a (200 mg, 0.48 mmol) in DCM (8 mL) were added 15b (335 mg, 0.81 mmol) and 4 Å molecular sieves (400 mg). The mixture was stirred at r.t. for 30 min. AgOTf (172 mg, 0.67 mmol) was added into the mixture and stirred at r.t. for 4 h. After complete reaction, the mixture was filtered and the filtrate was diluted by EA (100 mL), washed by saturated NaHCO₃ (50 mL*3) and brine (50 mL*3). The organic layer was dried over anhydrous Na₂SO₄, filtered and concentrated. The residue was purified by flash column chromatography (elute: PE/EA=100/0˜70/30) to provide 15c (230 mg, 64.2% yield) as white solid.

MS (ESI) m/z: 770.4 [M+Na]⁺

### Step 2

### (2R,3S,4S,5R,6R)-2-(acetoxymethyl)-6-((S)-2-amino-3-(benzyloxy)-3-oxopropoxy)tetrahydro-2H-pyran-3,4,5-triyl triacetate (15d)

To a solution of compound 15c (230 mg, 0.31 mmol) in DMF (6 mL) was added Et₂NH (450 mg, 602 mmol). The mixture was stirred at r.t. for 30 min. The mixture was concentrated under high vacuum and co-evaporated with toluene (3 mL*3) to give 15d (162 mg, crude) as brown solid.

MS (ESI) m/z: 526.4 [M+H]⁺

### Step 3

### (2R,3R,4S,5S,6R)-2-((S)-2-((S)-2-((((9H-fluoren-9-yl)methoxy)carbonyl)amino)-3-methylbutanamido)-3-(benzyloxy)-3-oxopropoxy)-6-(acetoxymethyl)tetrahydro-2H-pyran-3,4,5-triyl triacetate (15f)

To a solution of compound 15d (162 mg, 0.31 mmol) in DMF (4 mL) were added 15e (126 mg, 0.37 mol), HATU (176 mg, 0.46 mmol) and DIEA (120 mg, 0.93 mmol). The mixture was stirred at r.t. for 30 min. The mixture was concentrated and the residue was purified by flash column chromatography (elute: PE/EA=100/0˜70/30) to provide compound 15f (145 mg, 55.6% yield) as white solid.

MS (ESI) m/z: 869.5 [M+Na]⁺

### Step 4

### N-((((9H-fluoren-9-yl)methoxy)carbonyl)-L-valyl)-O-((2R,3R,4S,5S,6R)-3,4,5-triacetoxy-6-(acetoxymethyl)tetrahydro-2H-pyran-2-yl)-L-serine (15g)

To a solution of compound 15f (100 mg, 1.35 mmol) in MeOH (6 mL) was added Pd/C (10%, 20 mg). The mixture was stirred at H₂ atmosphere (15 psi) for 4 h. The mixture was filtered through a pad of celite, concentrated to give compound 15g (95 mg, crude) as white solid.

MS (ESI) m/z: 779.5 [M+Na]⁺

### Step 5

### (2R,3R,4S,5S,6R)-2-((S)-2-((S)-2-((((9H-fluoren-9-yl)methoxy)carbonyl)amino)-3-methylbutanamido)-3-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-6-(acetoxymethyl)tetrahydro-2H-pyran-3,4,5-triyl triacetate (15i)

To a solution of compound 15g (95 mg, crude) in DMF (4 mL) were added 15h (80 mg, 0.14 mol), HATU (72 mg, 0.19 mmol) and DIEA (49 mg, 0.38 mmol). The mixture was stirred at r.t. for 30 min. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min to provide 15i (117 mg, 70.4% yield) as white solid.

MS (ESI) m/z: 1340.5 [M+Na]⁺

### Step 6

### (2R,3S,4S,5R,6R)-2-(acetoxymethyl)-6-((S)-2-((S)-2-amino-3-methylbutanamido)-3-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)tetrahydro-2H-pyran-3,4,5-triyl triacetate (15j)

Compound 15j (116 mg, crude) was synthesized according to synthetic procedure of step 2 of example 15.

### Step 7

### (S)-2-amino-N—((S)-1-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)-3-methylbutanamide (15k)

To a solution of compound 15j (116 mg, 0.11 mmol) in MeOH (4 mL) was added K₂CO₃ (44 mg, 0.32 mmol). The mixture was stirred at r.t. for 3 h. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min to provide 15k (46 mg, 46.9% yield) as white solid.

MS (ESI) m/z: 928.6 [M+H]⁺

### Step 8

### 6-(2,5-dioxo-2,5-dihydro-1H-pyrrol-1-yl)-N-((10S,13S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-14-methyl-1,6,9,12-tetraoxo-10-((((2R,3R,4S,5R,6R)-3,4,5-trihydroxy-6-(hydroxymethyl)tetrahydro-2H-pyran-2-yl)oxy)methyl)-3-oxa-5,8,11-triazapentadecan-13-yl)hexanamide (15)

To a solution of compound 15i (9.5 mg, 0.042 mmol) in DMF (1.5 mL) were added HATU (14 mg, 0.038 mmol) and DIEA (5.4 mg, 0.042 mmol). The mixture was stirred at r.t. for 15 min. Compound 15k (23 mg, 0.021 mmol) was added into the mixture and was stirred at r.t. for 15 min. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min to provide 15k (3.4 mg, 14.4% yield) as white solid.

MS (ESI) m/z: 1143.5 [M+Na]⁺

### Example 16

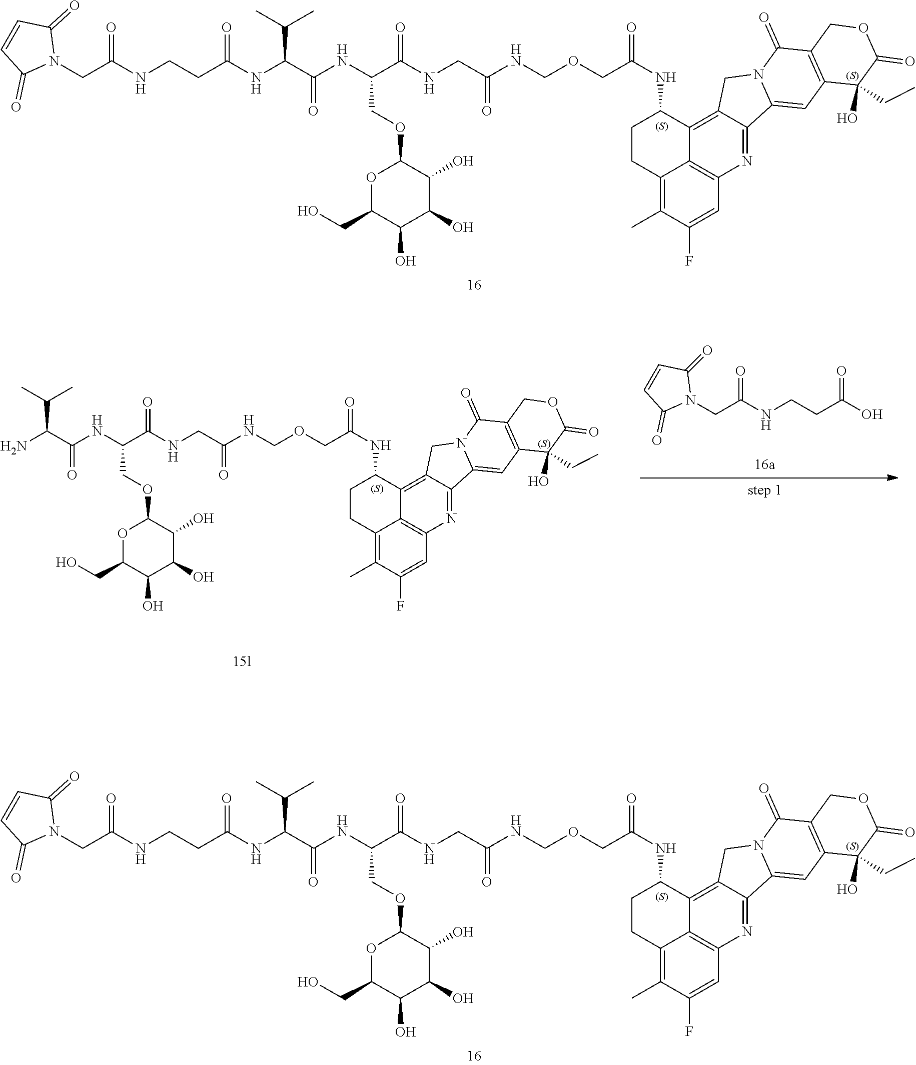

### Step 1

### (S)-2-(3-(2-(2,5-dioxo-2,5-dihydro-1H-pyrrol-1-yl)acetamido)propanamido)-N—((S)-1-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)-3-methylbutanamide (16)

Compound 16 (3.5 mg, 12.5% yield) was synthesized according to synthetic procedure of step 8 of example 15.

MS (ESI) m/z: 1158.5 [M+Na]⁺

### Example 17

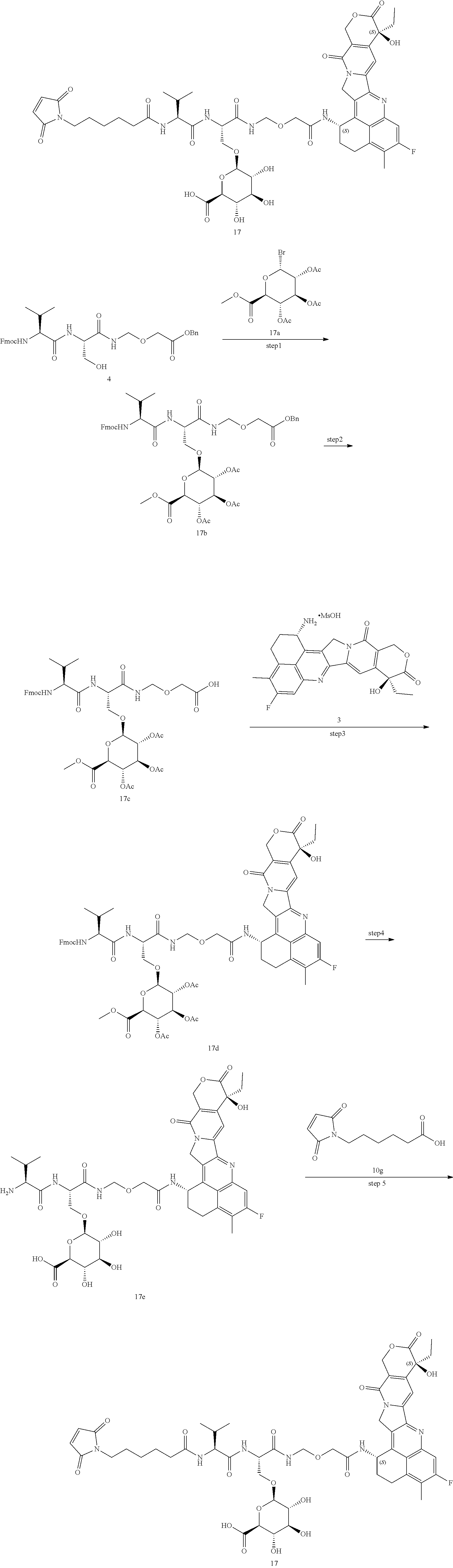

### Step 1

### (2R,3R,4S,5S,6S)-2-((S)-2-((S)-2-((((9H-fluoren-9-yl)methoxy)carbonyl)amino)-3-methylbutanamido)-3-(((2-(benzyloxy)-2-oxoethoxy)methyl)amino)-3-oxopropoxy)-6-(methoxycarbonyl)tetrahydro-2H-pyran-3,4,5-triyl triacetate (17b)

To the mixture of 4 (150 mg, 0.25 mmol), 17a (148 mg, 0.37 mmol) and 4 Å MS (800 mg) was added dry DCE (5 mL), stirred at r.t. for 30 min. AgOTf (83 mg, 0.32 mmol) was added under nitrogen atmosphere, stirred at r.t. for 14 h. The reaction solution was diluted with EtOAc (10 mL), filtered through Celite, washed with EtOAc (5 mL*3). The organic phase was washed with sat. NaHCO₃ (20 mL), concentrated and purified by flash column chromatography (eluent: petroleum ether/EtOAc=70/30 to 0/100 then DCM/MeOH=100/0 to 95/5) to give 17b (28 mg, 12.2% yield) as a light-yellow solid.

MS (ESI) m/z: 942.5 [M+Na]⁺

### Step 2

### (5S,8S)-1-(9H-fluoren-9-yl)-5-isopropyl-3,6,9-trioxo-8-((((2R,3R,4S,5S,6S)-3,4,5-triacetoxy-6-(methoxycarbonyl)tetrahydro-2H-pyran-2-yl)oxy)methyl)-2,12-dioxa-4,7,10-triazatetradecan-14-oic acid (17c)

To the mixture of 17b (114 mg, 0.12 mmol) and 10% Pd/C (26 mg, 0.012 mmol) was added MeOH (3 mL) under nitrogen atmosphere. The reaction solution was stirred at r.t. under H₂ atmosphere for 1 h. The solution was filtered and concentrated under vacuum to afford 17c (103 mg, 12.4% yield) as a white solid.

MS (ESI) m/z: 852.5 [M+Na]⁺

### Step 3

### (2R,3R,4S,5S,6S)-2-((S)-2-((S)-2-((((9H-fluoren-9-yl)methoxy)carbonyl)amino)-3-methylbutanamido)-3-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-3-oxopropoxy)-6-(methoxycarbonyl)tetrahydro-2H-pyran-3,4,5-triyl triacetate (17d)

To the solution of 17c (103 mg, 0.12 mmol), 3 (64 mg, 0.12 mmol) and HATU (47 mg, 0.12 mmol) in dry DMF (2 mL) was added DIPEA (61 μL, 0.36 mmol), stirred at r.t. for 1 h. The reaction solution was added into H₂O (20 mL), extracted with DCM/MeOH (10:1, 11 mL*3). The organic phase was concentrated and purified by flash column chromatography (eluent: DCM/MeOH=100/0 to 95/5) to afford 17d (97 mg, 64% yield) as a light brown solid.

MS (ESI) m/z: 1269.5 [M+Na]⁺

### Step 4

### (2S,3S,4S,5R,6R)-6-((S)-2-((S)-2-amino-3-methylbutanamido)-3-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-3-oxopropoxy)-3,4,5-trihydroxytetrahydro-2H-pyran-2-carboxylic acid (17e)

To the solution of 17d (30 mg, 0.024 mmol) in MeOH/H₂O (0.5/0.5 mL) was added TEA (167 μL, 1.2 mmol), stirred at r.t. for 9 h. The solution was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 17e (15 mg, 70.6% yield) as a pale-yellow solid.

MS (ESI) m/z: 885.5 [M+H]⁺

### Step 5

### (2S,3S,4S,5R,6R)-6-((S)-2-((S)-2-(6-(2,5-dioxo-2,5-dihydro-1H-pyrrol-1-yl)hexanamido)-3-methylbutanamido)-3-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-3-oxopropoxy)-3,4,5-trihydroxytetrahydro-2H-pyran-2-carboxylic acid (17)

To the solution of 10g (8.3 mg, 0.038 mmol) and HATU (11 mg, 0.029 mmol) in dry DMF (0.3 mL) was DIPEA (5 μL, 0.029 mmol), stirred at r.t. for 15 min. The solution was added dropwise into the solution of 17e (17 mg, 0.019 mmol) in dry DMF (0.7 mL), stirred at r.t. for 15 min. HOAc was added to adjust the pH to 5. The solution was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 17 (13 mg, 63.5% yield) as a beige solid.

MS (ESI) m/z: 1078.6 [M+H]⁺

### Example 18

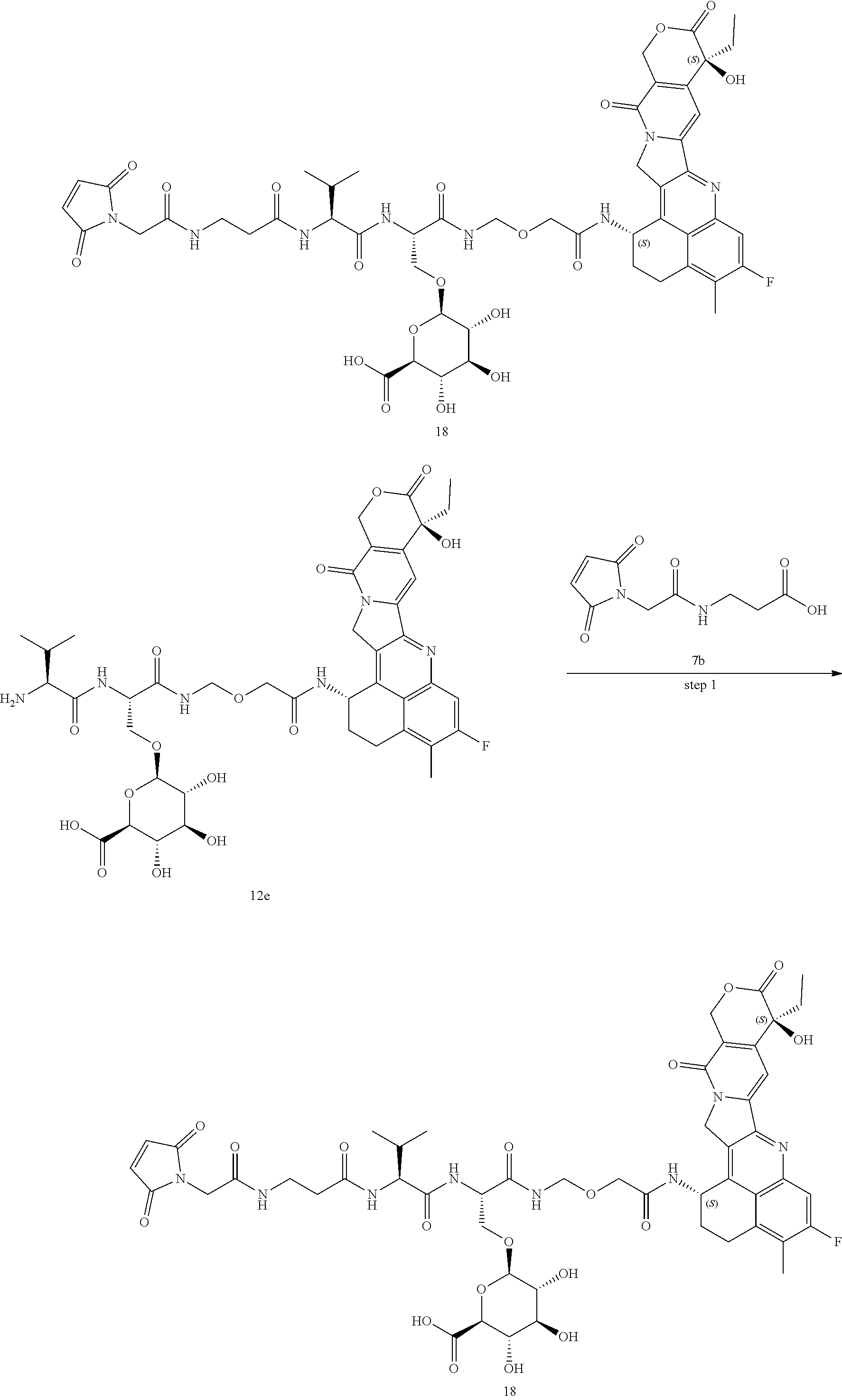

### Step 1

### (2S,3S,4S,5R,6R)-6-((S)-2-((S)-2-(3-(2-(2,5-dioxo-2,5-dihydro-1H-pyrrol-1-yl)acetamido)propanamido)-3-methylbutanamido)-3-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-3-oxopropoxy)-3,4,5-trihydroxytetrahydro-2H-pyran-2-carboxylic acid (18)

Compound 18 (2.9 mg, 15.7% yield) was synthesized according to synthetic procedure of step 5 of example 17.

MS (ESI) m/z: 1093.5 [M+H]⁺

### Example 19

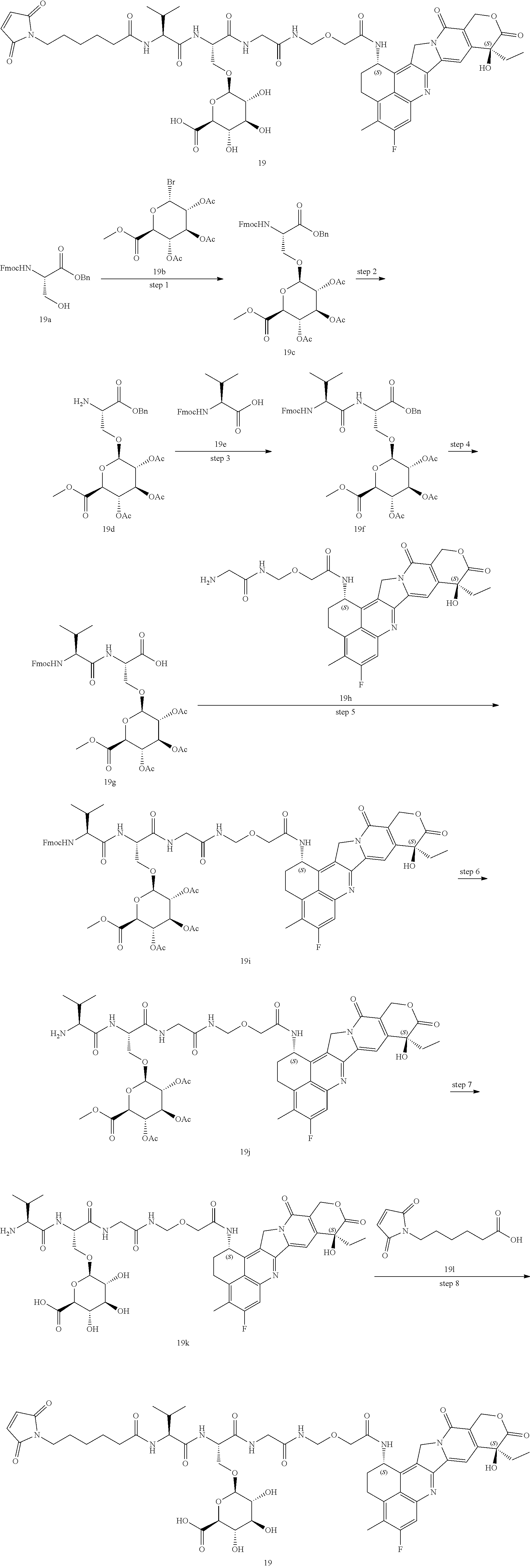

### Step 1

### (2R,3R,4S,5S,6S)-2-((S)-2-((((9H-fluoren-9-yl)methoxy)carbonyl)amino)-3-(benzyloxy)-3-oxopropoxy)-6-(methoxycarbonyl)tetrahydro-2H-pyran-3,4,5-triyl triacetate (19c)

To a mixture of 19a (834 mg, 2 mmol), 19b (950 mg, 2.4 mmol) and 4 Å MS (3000 mg) was added dry DCM (30 mL), stirred at r.t. for 30 min. AgOTf (617 mg, 2.4 mmol) was added under nitrogen atmosphere, stirred at r.t. for 16 h. The reaction solution was diluted with EtOAc (30 mL), filtered through Celite, washed with EtOAc (20 mL*3). The organic phase was washed with sat. NaHCO₃ (20 mL), concentrated and purified by flash column chromatography (eluent: petroleum ether/EtOAc=70/30 to 0/100) to give 19c (470 mg, 32.1% yield) as a light-yellow solid.

MS (ESI) m/z: 756.3 [M+Na]⁺

### Step 2

### (2R,3R,4S,5S,6S)-2-((S)-2-amino-3-(benzyloxy)-3-oxopropoxy)-6-(methoxycarbonyl)tetrahydro-2H-pyran-3,4,5-triyl triacetate (19d)

To a solution of 19c (470 mg, 0.64 mmol) in DMF (5 mL) was added Et₂N (1420 uL, 1011 mg, 12.8 mmol). The mixture was stirred at r.t. for 0.5 h. On completion of the reaction. The mixture was concentrated under vacuum to give 19d (327 mg, crude) as a yellow solid.

MS (ESI) m/z: 512.4 [M+H]⁺

### Step 3

### (2R,3R,4S,5S,6S)-2-((S)-2-((S)-2-((((9H-fluoren-9-yl)methoxy)carbonyl)amino)-3-methylbutanamido)-3-(benzyloxy)-3-oxopropoxy)-6-(methoxycarbonyl)tetrahydro-2H-pyran-3,4,5-triyl triacetate (19f)

To a solution of 19d (327 mg) in DMF (5 mL) were added HATU (291 mg, 0.76 mmol) and DIPEA (223 μL, 165 mg, 1.27 mmol). The resulted yellow solution was stirred at r.t. for 5 min then 19e (239 mg, 0.70 mmol) was added. The mixture was stirred at r.t. for 60 min. On completion of the reaction. Solvent was evaporated and the residue was purified by flash column chromatography (eluent: petroleum ether/EtOAc=70/30 to 0/100) and prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 19f (300 mg, 56.2% yield) as a white solid.

MS (ESI) m/z: 833.4 [M+H]⁺

### Step 4

### N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serine (19g)

To a solution of 19f (300 mg, 0.36 mmol) in MeOH (10 mL) and THF (8 mL) was added wet Pd/C (30 mg) then purge with H₂ balloon for 3 times and stirred at r.t. for 2 h. On completion of the reaction. The mixture was filtered Pd/C through syringe filter head and the filtrate was concentrated under vacuum to give 19g (265 mg, crude) as a white solid.

MS (ESI) m/z: 765.3 [M+Na]⁺

### Step 5

### (2R,3R,4S,5S,6S)-2-((S)-2-((S)-2-((((9H-fluoren-9-yl)methoxy)carbonyl)amino)-3-methylbutanamido)-3-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-6-(methoxycarbonyl)tetrahydro-2H-pyran-3,4,5-triyl triacetate (19i)

To a solution of 19g (265 mg) in DMF (5 mL) were added HATU (120 mg, 0.42 mmol) and DIPEA (124 μL, 92 mg, 0.71 mmol). The resulted yellow solution was stirred at r.t. for 5 min then 19h (227 mg, 0.39 mmol) was added. The mixture was stirred at r.t. for 60 min. On completion of the reaction. Solvent was evaporated and the residue was purified by flash column chromatography (eluent: DCM/MeOH=95/5 to 90/10) to give 19i (280 mg, 59.6% yield) as a brown solid.

MS (ESI) m/z: 1326.4 [M+Na]⁺

### Step 6

### (2R,3R,4S,5S,6S)-2-((S)-2-((S)-2-amino-3-methylbutanamido)-3-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-6-(methoxycarbonyl)tetrahydro-2H-pyran-3,4,5-triyl triacetate (19j)

To a solution of 19i (280 mg, 0.21 mmol) in DMF (5 mL) was added Et₂N (478 uL, 340 mg, 4.29 mmol). The mixture was stirred at r.t. for 0.5 h. On completion of the reaction. The mixture was concentrated under vacuum to give 19j (220 mg, crude) as a white solid.

MS (ESI) m/z: 1082.4 [M+H]⁺

### Step 7

### (2S,3S,4S,5R,6R)-6-((S)-2-((S)-2-amino-3-methylbutanamido)-3-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-3,4,5-trihydroxytetrahydro-2H-pyran-2-carboxylic acid (19k)

To the solution of 19j (220 mg) in MeOH/H₂O (5/5 mL) was added Na₂CO₃ (151 mg, 1.42 mmol), stirred at r.t. for 9 h. The solution was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 19k (45 mg, 23.8% yield) as a white solid.

MS (ESI) m/z: 942.3 [M+H]⁺

### Step 8

### (2S,3S,4S,5R,6R)-6-((S)-2-((S)-2-(6-(2,5-dioxo-2,5-dihydro-1H-pyrrol-1-yl)hexanamido)-3-methylbutanamido)-3-((2-(((2-(((1R,9R)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-3,4,5-trihydroxytetrahydro-2H-pyran-2-carboxylic acid (19)

To the solution of 191 (5.76 mg, 0.026 mmol) and HATU (9.576 mg, 0.025 mmol) in dry DMF (1 mL) was DIPEA (7.3 μL, 5.4 mg, 0.042 mmol), stirred at r.t. for 15 min. The solution was added dropwise into the solution of 19k (20 mg, 0.021 mmol) in dry DMF (1 mL), stirred at r.t. for 15 min. The solution was purified by prep-HPLC (FA) (Method: column: XBridge Prep C18 OBD 5 μm 19*250 mm; Mobile phase: A—water (0.1% formic acid): B—acetonitrile; Flow rate: 20 mL/min, the fraction was lyophilized to give 19 (7.4 mg, 31.0% yield) as a white solid.

MS (ESI) m/z: 1135.4 [M+H]⁺

### Example 20

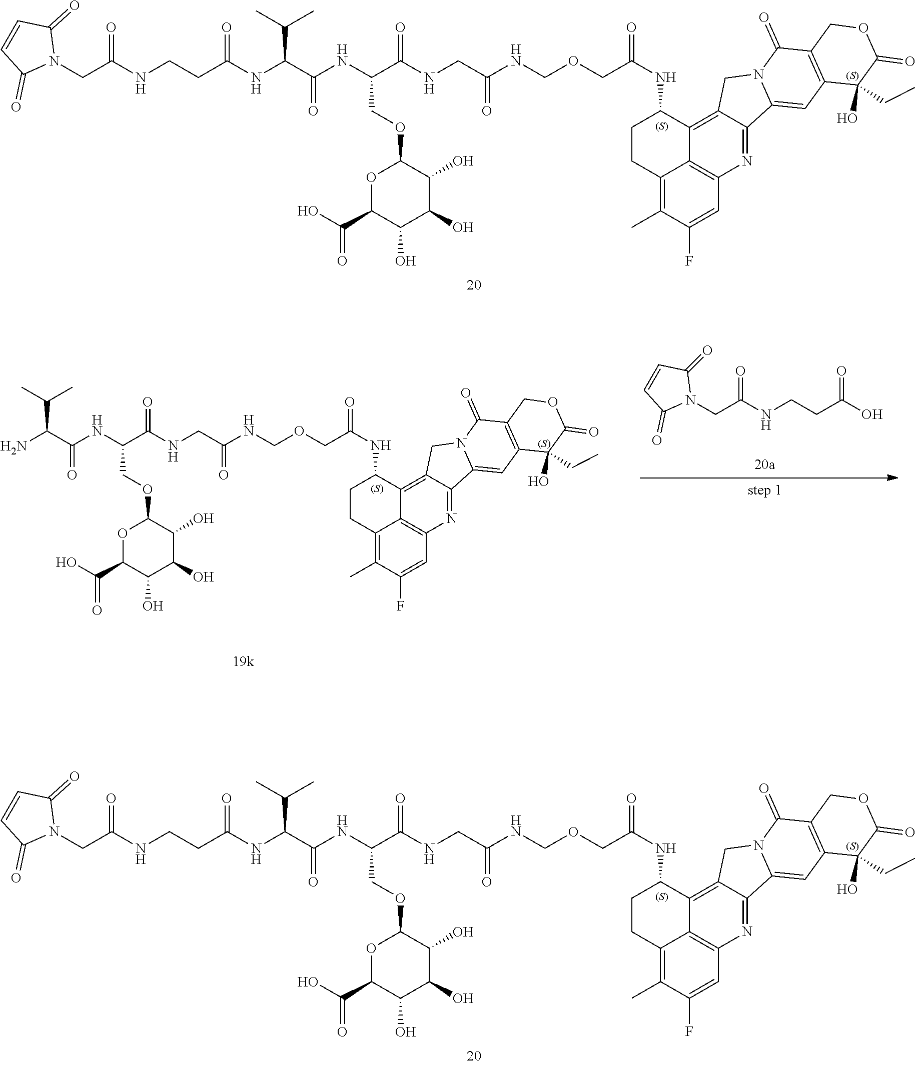

### Step 1

### (2S,3S,4S,5R,6R)-6-((S)-2-((S)-2-(3-(2-(2,5-dioxo-2,5-dihydro-1H-pyrrol-1-yl)acetamido)propanamido)-3-methylbutanamido)-3-((2-(((2-(((1R,9R)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-3,4,5-trihydroxytetrahydro-2H-pyran-2-carboxylic acid (20)

Compound 20 (12 mg, 50.1% yield) was synthesized according to synthetic procedure of step 8 of example 19.

MS (ESI) m/z: 1172.4 [M+Na]⁺

### ADC Preparation and Characterization

### Antibody Drug Conjugate Preparation

### DAR8 Antibody Drug Conjugate Preparation

Antibody in conjugation buffer (with concentration 0.5-25 mg/mL, PBS buffer pH 6.0-8.5) was incubated under reduction temperature (0-40° C.) for 10 min and 8-15 eq. TECP solution (5 mM stock in PBS buffer) was added into the reaction mixture and left the reduction reaction for 1-8 hours at reduction temperature. Organic solvent (eg: DMSO, DMF, DMA, PG, acetonitrile, 0-25% v/v) and linker-payload stock (10-25 eq, 10 mM stock in organic solvent) were added stepwise after reduction mixture was cooled down to 0-25° C. Conjugation solution was left for 1-3 h at 0-25° C. and the reaction can be quenched with N-acetyl Cysteine (1 mM stock). The solution was submitted to buffer exchange (spin desalting column, ultrafiltration, and dialysis) into storage buffer (for example: pH 5.5-6.5 Histidine acetate buffer, with optional additive such as sucrose, trehalose, tween 20, 60, 80).

Maleimide Hydrolysis after Conjugation

After the conjugation step, the ADC was undergone buffer exchange into ring opening buffer (pH 8.0˜9.0, borate or Tris buffer) and the solution was left at 22 or 37° C. for 5˜48 h. Ring opening process was monitored via reduced LCMS. Once the conjugated maleimide hydrolysis is completed, the resulting ADCs were buffer exchanged into basic Tris pH 8.0-8.5 buffer or acidic histidine-acetate pH 5.0-6.5 buffer via dialysis.

### ADC Characterization

ADC examples were prepared by following above procedures with DAR 8 profile. All ADCs were characterized via following analytical methods.

Drug to antibody ratio (DAR) of the ADCs were determined by LCMS method or HIC method.

SEC purity of ADCs made are all >95% purity.

### Drug to Antibody Ratio (DAR) Determination

### LCMS Method

LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 μg
MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

### HIC Method

HPLC analysis was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM Phosphate buffer, pH 7.0
Mobile phase B: 50 mM Phosphate buffer, 25% (V/V) Isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

### ADC Purity

### SEC Method

HPLC analysis was carried out under the following measurement conditions:
HPLC system: Waters H-Class UPLC System
Detector: measurement wavelength: 280 nm
Column: ACQUITY UPLC BEH200 SEC 1.7 um 4.6×150 mm, Waters
Column temperature: room temperature
Mobile phase A: 200 mM Phosphate buffer, 250 mM potassium chloride, 15% isopropyl alcohol, PH 7.0
Gradient program: under 10 min isocratic elutions with the flow rate of 0.3 mL/min
Injected sample amount: 20 μg

### ADC Hydrophobicity Evaluation

ADC with more hydrophobic property would appeared with later retention time from HIC (hydrophobicity interaction column) chromatography. The DAR8 (antibody with 8 drug-loading) peak of the example ADCs for this comparison.

By comparing HIC D8 RT, it is clearly to tell that ADC hydrophilicity ranks as below ADC-C4>ADC-C5>ADC-C2>ADC-C3>ADC-C1. ADC-C1 appears as the most hydrophobic property in this set.

### HIC Method

### Method 1

HPLC analysis was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM Phosphate buffer, pH 7.0
Mobile phase B: 50 mM Phosphate buffer, 25% (V/V) Isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

### Method 2

HPLC analysis was carried out under the following measurement conditions:

HPLC system: Waters ACQUITY ARC HPLC System
Detector: measurement wavelength: 280 nm
Column: MABPac HIC-10, 5 μm, 4.6×10 mm (Thermo)
Column temperature: 25° C.
Mobile phase A: 1.5 M ammonium sulfate, 50 mM sodium phosphate, pH 7.0
Mobile phase B: 50 mM sodium phosphate, pH 7.0
Gradient program: 20% B—20% B (0 min-1 min), 0% B-0% B (1 min-35 min), 20% B-20% B (35 min-40 min)
Flow rate: 0.5 mL/min
Sample preparation: The sample was diluted with initial mobile phase to 0.5 mg/mL.

TABLE 1
ADC Examples (with structures)

| | | | DAR8 peak |
|---|---|---|---|
| | | | HIC |
| | | | retention |
| Example | Antibody | ADC structure | time (min) |
| ADC-C1 | Ifinatamab | 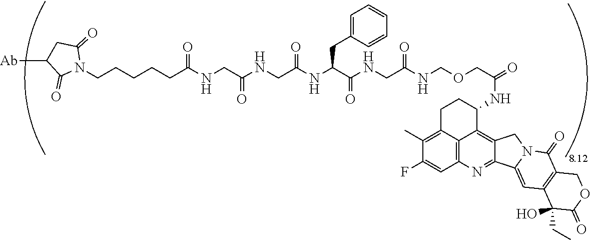 | Method 1: 10.860 |
| ADC-C2 | Ifinatamab | 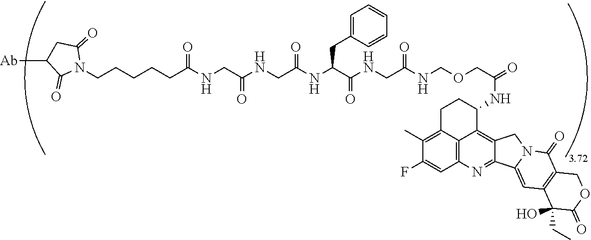 | Method 1: 10.860 |
| ADC-C3 | Ifinatamab | 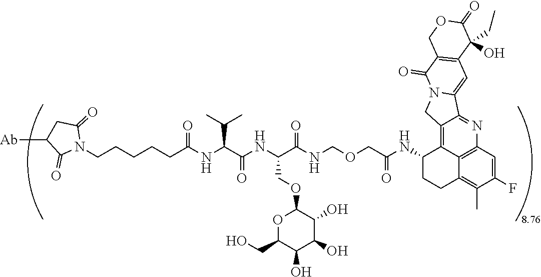 | Method 1: 9.725 |
| ADC-C4 | Ifinatamab | 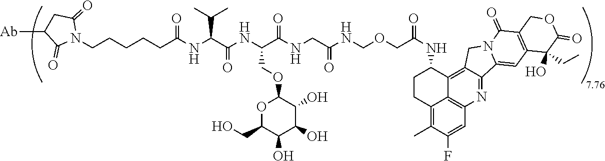 | Method 1: 9.764 |
| ADC-C5 | Ifinatamab | 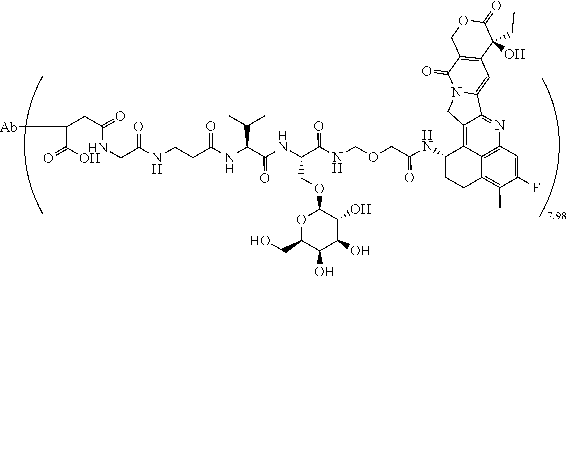 | Method 1: 9.898 |
| ADC-C6 | Ifinatamab | 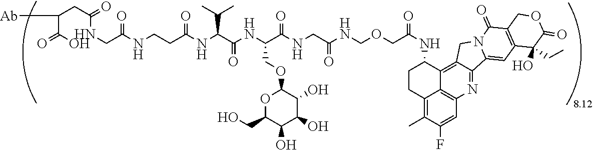 | Method 1: 9.970 |
| ADC-C7 | Ifinatamab | 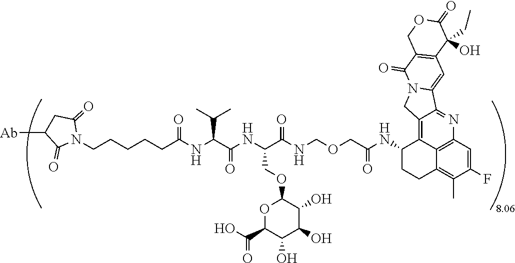 | Method 1: 9.675 |
| ADC-C8 | Ifinatamab | 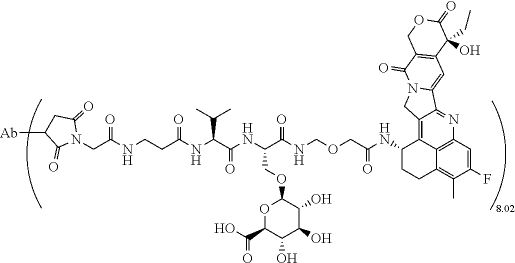 | Method 1: 9.676 |
| ADC-C9 | Ifinatamab | 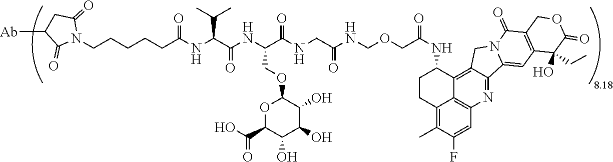 | Method 1: 9.698 |
| ADC- C10 | Ifinatamab | 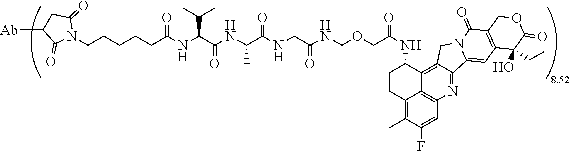 | Method 1: 10.309 |
| ADC- C11 | Ifinatamab | 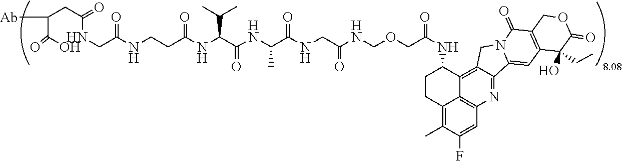 | Method 1: 10.299 |
| ADC- C12 | Ifinatamab | 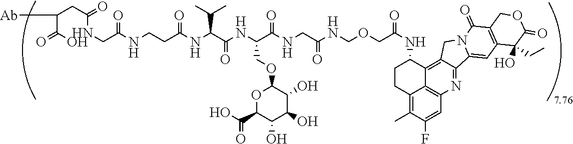 | Method 1: 9.764 |
| ADC- C13 | Ifinatamab | 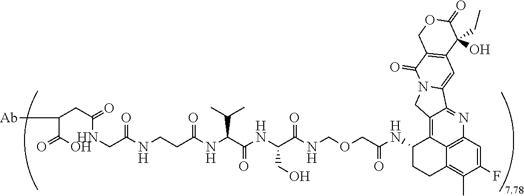 | Method 1: 10.105 |
| ADC- C14 | Ifinatamab | 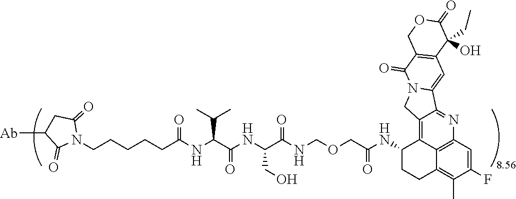 | Method 1: 10.235 |

All ADCs appear to be more hydrophilic than ADC-C1 base on DAR8 species retention time From HIC profiles.

### Primary Sequence of Ifinatamab

Heavy chain (SEQ ID NO: 1):
QVQLVQSGAEVKKPGSSVKVSCKASGYTFTNYVMHWVRQAPGQGLEWMGY
INPYNDDVKYNEKFKGRVTITADESTSTAYMELSSLRSEDTAVYYCARWG
YYGSPLYYFDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
Light Chain (SEQ ID NO: 2):
EIVLTQSPATLSLSPGERATLSCRASSRLIYMHWYQQKPGQAPRPLIYAT
SNLASGIPARFSGSGSGTDFTLTISSLEPEDFAVYYCQQWNSNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

### ADC Cellular Killing Assay

Cell line information

| Cell | Resource | TAA level |
|---|---|---|
| H358 | ATCC | High |
| H441 | ATCC | Medium |
| H1048 | ATCC | Medium to low |
| MDA-MB-453 | SIBS | Negative |

### Assay Method

Seeding cells H358 (1E3/well), H441 (1E3/well), H1048 (1E3/well) and MDA-MB-453 (1E3/well) into 3D-96-well plate (Corning: 4520), 80 ul/well. Incubation at 37° C., 5% CO₂, overnight.
Adding the fresh growth-medium containing the varying concentrations of ADCs, 40 ul/well into H358, H441, H1048 and MDA-MB-453.
Incubation at 37° C., 5% CO₂, 6 days.
The H358, H441, H1048 and MDA-MB-453 cell viability was detected by 3D reagent (Promega, G9683), 100 ul/well of 3D reagent.
Incubate the 3D-plates at room temperature for 30 minutes to stabilize luminescent signal.
Analyze with Microplate Reader.

### Cellular Assay Killing Results

Data set 1

| | H358 | | H441 | | H1048 | | MDA-MB-453 | |
|---|---|---|---|---|---|---|---|---|
| | | Max | | Max | | Max | | Max |
| | | kill- | | kill- | | kill- | | kill- |
| | EC50 | ing | EC50 | ing | EC50 | ing | EC50 | ing |
| Example | (nM) | (%) | (nM) | (%) | (nM) | (%) | (nM) | (%) |
| ADC-C1 | 0.022 | 72 | 2.201 | 100 | 0.034 | 96 | >100 | / |
| ADC-C10 | 0.025 | 66 | 1.073 | 100 | 0.029 | 96 | 22 | / |
| ADC-C11 | 0.027 | 72 | 1.234 | 100 | 0.025 | 94 | >100 | / |
| ADC-C13 | 0.01 | 73 | 0.861 | 100 | 0.014 | 95 | >100 | / |
| ADC-C14 | 0.033 | 64 | 0.511 | 100 | 0.056 | 100 | >100 | / |

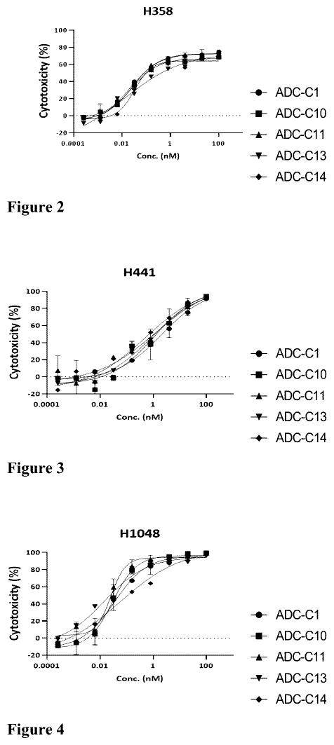

With tandem release linker design, the cellular killings of ADC-C1, ADC-C10, ADC-C11, ADC-C13, and ADC-C14 appear to be similar to ADC-C1 indicating efficient payload release within cells (FIG. 2, FIG. 3, FIG. 4, and FIG. 5).

Data set 2

| | H358 | | H441 | | H1048 | | MDA-MB-453 | |
|---|---|---|---|---|---|---|---|---|
| | | Max | | Max | | Max | | Max |
| | | kill- | | kill- | | kill- | | kill- |
| | EC50 | ing | EC50 | ing | EC50 | ing | EC50 | ing |
| Example | (nM) | (%) | (nM) | (%) | (nM) | (%) | (nM) | (%) |
| ADC-C1 | 0.022 | 72 | 3.764 | 100 | 0.034 | 96 | >100 | / |
| ADC-C3 | 0.018 | 74 | 3.291 | 100 | 0.008 | 97 | >100 | / |
| ADC-C4 | 0.022 | 61 | 2.291 | 94 | 0.018 | 100 | >100 | / |
| ADC-C5 | 0.074 | 60 | 1.245 | 95 | 0.038 | 98 | >100 | / |
| ADC-C6 | 0.038 | 65 | 0.5298 | 96 | 0.013 | 95 | >100 | / |

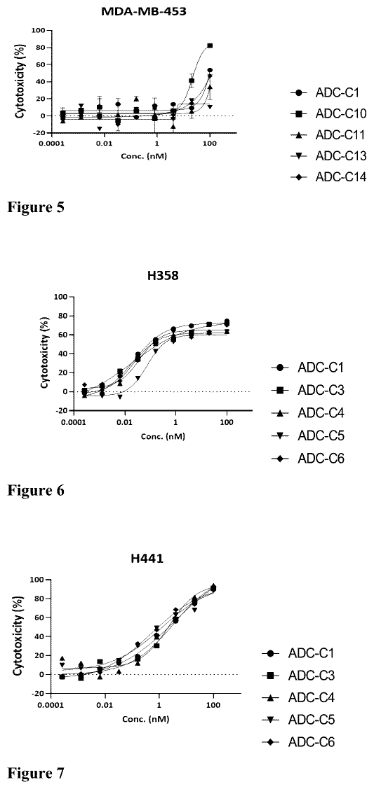

With tandem release linker design, the cellular killings of ADC-C, ADC-C3, ADC-C4, ADC-C5, and ADC-C6 appear to be similar to ADC-C1 indicating efficient payload release within cells (FIG. 6, FIG. 7, FIG. 8, and FIG. 9).

Data set 3

| | H358 | | H441 | | H1048 | | MDA-MB-453 | |
|---|---|---|---|---|---|---|---|---|
| | | Max | | Max | | Max | | Max |
| | | kill- | | kill- | | kill- | | kill- |
| | EC50 | ing | EC50 | ing | EC50 | ing | EC50 | ing |
| Example | (nM) | (%) | (nM) | (%) | (nM) | (%) | (nM) | (%) |
| ADC-C1 | 0.022 | 72 | 2.087 | 100 | 0.034 | 99 | >100 | / |
| ADC-C7 | 0.038 | 64 | 0.894 | 100 | 0.007 | 97 | >100 | / |
| ADC-C8 | 0.043 | 68 | 1.042 | 100 | 0.015 | 94 | >100 | / |
| ADC-C9 | 0.033 | 68 | 1.647 | 100 | 0.024 | 98 | >100 | / |
| ADC-C12 | 0.029 | 63 | 1.094 | 100 | 0.034 | 99 | >100 | / |

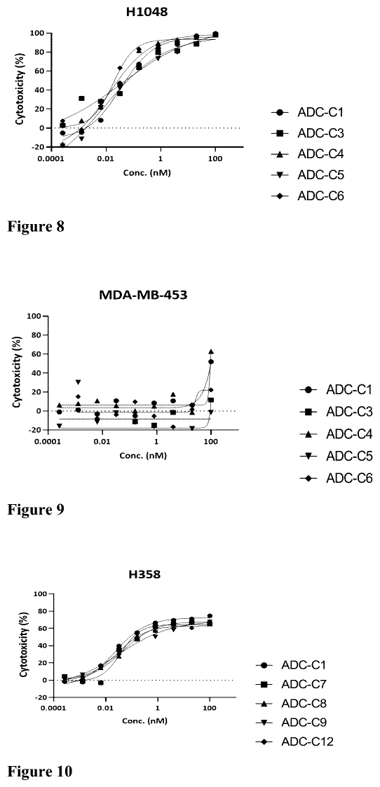

With tandem release linker design, the cellular killings of ADC-C1, ADC-C7, ADC-C8, ADC-C9, and ADC-C12 appear to be similar to ADC-C1 indicating efficient payload release within cells (FIG. 10, FIG. 11, FIG. 12, and FIG. 13).

ADC enzymatic release study

| Name | Vender | Cat No. | Lot No. | Units | |
|---|---|---|---|---|---|
| Cathepsin B from | Sigma | C8571-25UG | SLCH4801 | 1500 | units/mg |
| human liver | | | | | |
| Mixed Gender | XENOTECH | H0610.L-0.25 mL | — | 2.5 | mg/mL |
| Liver Lysosomes | | | | | |

### Assay Protocol

1. A 50 mM sodium acetate solution (pH 5.0) was prepared, and then TCEP was added to make the concentration of TCEP 2 mM to obtain the reaction solution
2. A reaction solution was used to dilute ADC and Lysosome to a final concentration of 1 uM for ADC and 0.125 mg/mL for Lysosome (or 20 units/mL of Cathepsin-B). 5 copies of the sample were prepared in parallel for each ADC with a final volume of 100 uL
3. The samples prepared in parallel were incubated at 37° C., and 200 μL of cold acetonitrile (including internal standard) was added at 0 h, 0.5 h, 1 h, 2 h and 6 h to terminate the reaction
4. After centrifugation, 200 uL supernatant was taken for LC-MS analysis to detect the free payload and calculate the payload release rate.

Payload release rate (%)=(the concentration of DXd in supernatant/the MW of DXd)/(the concentration of ADC/the MW of ADC)*DAR*100

### Results

### Group 1

### Payload Release Under Lysosome Treatment:

| | DXd release rate (%) under lysosome treatment | | | | |
|---|---|---|---|---|---|
| Example | 0 h | 0.5 h | 1 h | 2 h | 6 h |
| ADC-C1 | 0.5 | 8.26 | 13.28 | 23.35 | 49.70 |
| ADC-C10 | 0 | 0.04 | 0.07 | 0.15 | 0.29 |
| ADC-C11 | 0.03 | 0.05 | 0.06 | 0.11 | 0.36 |
| ADC-C13 | 0.15 | 1.40 | 3.28 | 8.30 | 24.77 |
| ADC-C14 | 0.23 | 8.23 | 14.10 | 26.26 | 55.94 |

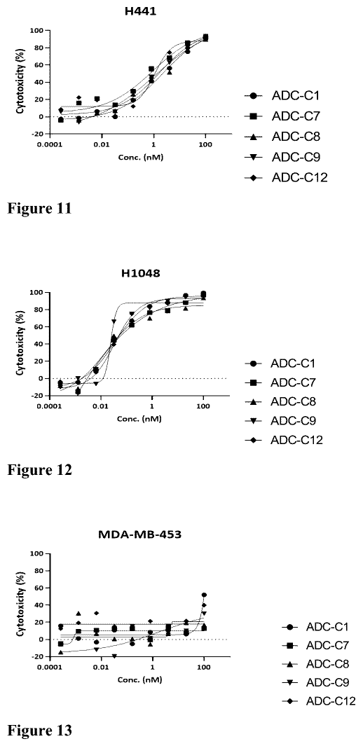

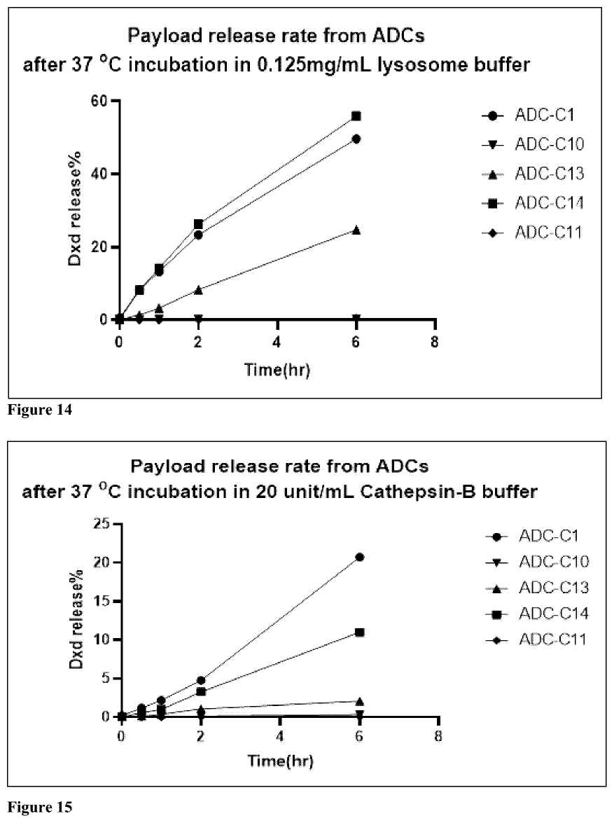

Payload release profile of ADC-C1, ADC-C10, ADC-C11, ADC-C13, and ADC-C14 under lysosome treatment can refer to FIG. 14.

The payload release rate of ADC-C14 and ADC-C1 are faster than ADC-C13 and much faster than ADC-C10 and ADC-C11 in lysosome.

### Payload Release Under Cathepsin B Treatment:

| | DXd release rate (%) under Cathepsin B treatment | | | | |
|---|---|---|---|---|---|
| Example | 0 h | 0.5 h | 1 h | 2 h | 6 h |
| ADC-C1 | 0.19 | 1.17 | 2.18 | 4.76 | 20.74 |
| ADC-C10 | 0.03 | 0.06 | 0.08 | 0.16 | 0.32 |
| ADC-C11 | 0.03 | 0.05 | 0.06 | 0.11 | 0.36 |
| ADC-C13 | 0.04 | 0.11 | 0.36 | 1.08 | 2.07 |
| ADC-C14 | 0.06 | 0.56 | 1.01 | 3.25 | 11.00 |

Payload release profile of ADC-C1, ADC-C1, ADC-C11, ADC-C13, and ADC-C14 under Cathepsin B treatment can refer to FIG. 15.

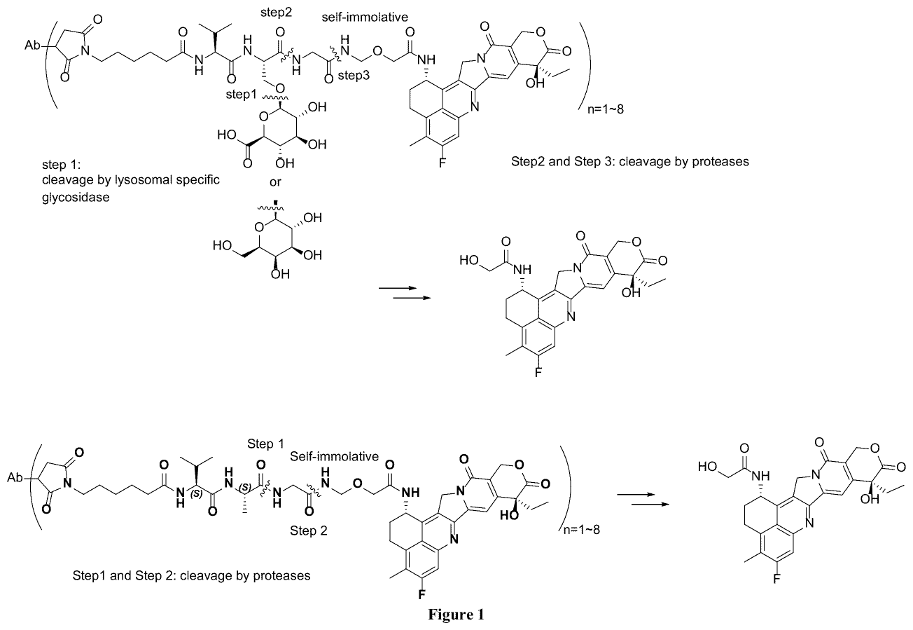

The payload release rate of ADC-C1 is faster than ADC-C14 and much faster than ADC-C13, ADC-C10 and ADC-C11 under cathepsin B treatment. A tandem release fashion of ADCs was shown in FIG. 1.

### Group 2

Payload release under lysosome treatment:

| | DXd release rate (%) under lysosome treatment | | | | |
|---|---|---|---|---|---|
| Example | 0 h | 0.5 h | 1 h | 2 h | 6 h |
| ADC-C1 | 0.5 | 8.26 | 13.28 | 23.35 | 49.70 |
| ADC-C3 | 0.23 | 2.39 | 5.58 | 13.75 | 40.92 |
| ADC-C4 | 0.03 | 0.03 | 0.03 | 0.06 | 0.33 |
| ADC-C5 | 0.15 | 0.27 | 0.51 | 1.8 | 5.13 |
| ADC-C6 | 0.00 | 0.00 | 0.03 | 0.05 | 0.18 |

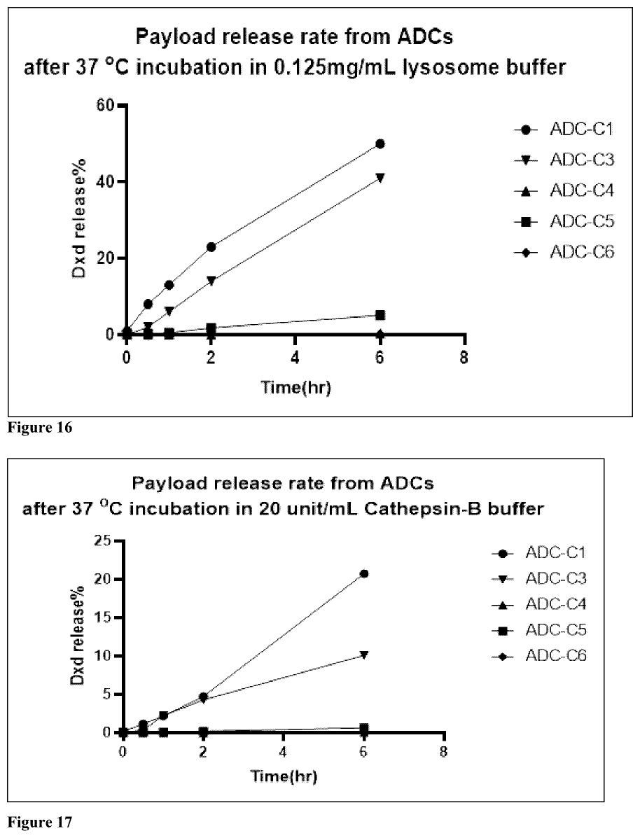

Payload release profile of ADC-C1, ADC-C3, ADC-C4, ADC-C5, and ADC-C6 under lysosome treatment can refer to FIG. 16.

The payload release rate of ADC-C1 and ADC-C3 are faster than ADC-C5 and much faster than ADC-C4 and C6 under lysosome treatment. It was suspected that deconjugation event of ADC-C3 may occur under this lysosome assay and the unconjugated linker-payload is more vulnerable toward enzymatic cleavage than the conjugated one. Therefore, more payload release was observed in ADC-C3 than ADC-C5. Although ADC-C4 and ADC-C6 demonstrated slower DXd payload release rate under this assay condition, both ADCs have demonstrated comparable cellular killing activities with all ADCs indicating efficient payload release did occur in in vitro cellular setting. A tandem release fashion of ADCs was shown in FIG. 8.

### Payload Release Under Cathepsin B Treatment:

| | DXd release rate (%) under Cathepsin B treatment | | | | |
|---|---|---|---|---|---|
| Example | 0 h | 0.5 h | 1 h | 2 h | 6 h |
| ADC-C1 | 0.19 | 1.17 | 2.18 | 4.76 | 20.74 |
| ADC-C3 | 0.06 | 0.37 | 2.24 | 4.31 | 10.12 |
| ADC-C4 | 0.00 | 0.00 | 0.00 | 0.02 | 0.03 |
| ADC-C5 | 0.08 | 0.10 | 0.15 | 0.21 | 0.65 |
| ADC-C6 | 0.00 | 0.00 | 0.02 | 0.03 | 0.05 |

Payload release profile of ADC-C1, ADC-C3, ADC-C4, ADC-C5, and ADC-C6 under Cathepsin B treatment can refer to FIG. 17.

The payload release rate of ADC-C1 is faster than ADC-C3 and much faster than ADC-C5, ADC-C4 and ADC-C6 under cathepsin B treatment. Abundant b-Galactosidase exists in lysosome and recognizes b-galactose moiety to trigger glycan C—O bond cleavage (J. Med. Chem. 2006, 49, 6290-6297, Chem. Commun. 2021 Spring D. R. et al). A tandem release fashion of ADCs was shown in FIG. 8.

### Group 3

### Payload Release Under Lysosome Treatment:

| | DXd release rate (%) under lysosome treatment | | | | |
|---|---|---|---|---|---|
| Example | 0 h | 0.5 h | 1 h | 2 h | 6 h |
| ADC-C1 | 0.5 | 8.26 | 13.28 | 23.35 | 49.70 |
| ADC-C7 | 0.1 | 0.23 | 0.61 | 2.11 | 16.01 |
| ADC-C8 | 0.07 | 0.09 | 0.17 | 0.63 | 5.60 |
| ADC-C9 | 0.02 | 0.03 | 0.05 | 0.08 | 0.38 |
| ADC-C12 | 0.03 | 0.04 | 0.06 | 0.06 | 0.15 |

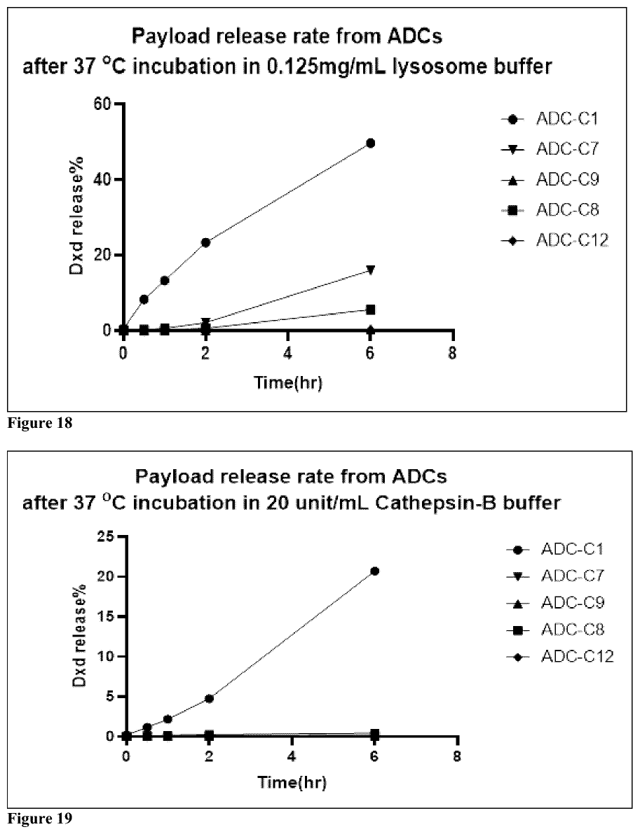

Payload release profile of ADC-C1, ADC-C7, ADC-C8, ADC-C9, and ADC-C12 under lysosome treatment can refer to FIG. 18.

The payload release rate of ADC-C1 is faster than ADC-C7 and much faster than ADC-C8, ADC-C9 and C12 under lysosome treatment. It was suspected that deconjugation event of ADC-C7 may occur under this lysosome assay and the unconjugated linker-payload is more vulnerable toward enzymatic cleavage than the conjugated one. Therefore, more payload release was observed in ADC-C7 than ADC-C8. Although ADC-C8, ADC-C9, ADC-02 demonstrated slower DXd payload release rate under this assay condition, both ADCs have demonstrated comparable cellular killing activities with all ADCs indicating efficient payload release did occur in in vitro cellular setting. A tandem release fashion of ADCs was shown in FIG. 1.

### Payload Release Under Cathepsin B Treatment:

| | DXd release rate (%) under Cathepsin B treatment | | | | |
|---|---|---|---|---|---|
| Example | 0 h | 0.5 h | 1 h | 2 h | 6 h |
| ADC-C1 | 0.19 | 1.17 | 2.18 | 4.76 | 20.74 |
| ADC-C7 | 0.04 | 0.10 | 0.16 | 0.23 | 0.45 |
| ADC-C8 | 0.04 | 0.05 | 0.06 | 0.09 | 0.18 |
| ADC-C9 | 0.00 | 0.00 | 0.00 | 0.03 | 0.06 |
| ADC-C12 | 0.00 | 0.00 | 0.00 | 0.03 | 0.06 |

Payload release profile of ADC-C1, ADC-C7, ADC-C8, ADC-C9, and ADC-C12 under Cathepsin B treatment can refer to FIG. 19.

The payload release rate of ADC-C1 is much faster than ADC-C7, ADC-C8, ADC-C9 and ADC-C12 under cathepsin B treatment. Abundant glucuronidase exists in lysosome and recognizes b-galactose moiety to trigger glycan C—O bond cleavage under acidic condition (Euro. J. Med. Chem. 2014 Papot S. et al.; Mol. Can. Ther. 2016 Lyon R. P. et al.). A tandem release fashion of ADCs was shown in FIG. 8.

### ADC Plasma Stability

Incubation of ADC with Plasma
Dilute ADC into mouse or human plasma to yield a final solution of 100 μg/mL ADC in plasma
Incubate the samples at 37° C.
Aliquots (100 μL) were taken at four time points (0, 4, 24, 72, 96, or 168 h)
Samples are frozen at −80° C. until analysis.
   Plasma Payload Concentrations were Carried Out Under the Following Measurement Conditions:
Instrument: LC-MS/MS (Triple Quad 5500)
Monitor: MRM
Column: Advanced Materials Technology, HALO AQ-C18 2.7 μm 90 Å, 50*2.1 mm
Column temperature: 40° C.
Mobile phase A: H2O-0.1% FA
Mobile phase B: ACN-0.11% FA
Gradient program for DXd: 20% B-20% B (0 min-0.2 min), 20% B-80% B (0.2 min-1.5 min), 80% B-80% B (1.5 min-2.2 min), 80% B-20% B (2.20 min-2.21 min), 20% B-20% B (2.21 min-3.0 min); Gradient program for Dxd: 2% B-2% B (0 min-0.2 min), 2% B-98% B (0.2 min-1.2 min), 98% B-98% B (1.2 min-2.0 min), 98% B-2% B (2.0 min-2.01 min), 2% B-2% B (2.01 min-4.0 min);
Injected sample amount: 10 μL
   Plasma ADC and Total Ab(Tab) Concentrations were Carried Out Under the Following Measurement Conditions:
Assay: Ligand binding assay (ELISA)
Capture reagent: B7H3 ECD
Detection reagent: anti-payload Ab for ADC and anti-human IgG polyclonal Ab for Total Ab.

### Results

### Payload Release in Human Plasma

### Group 1

| | DXd release rate (%) under human plasma treatment | | | | |
|---|---|---|---|---|---|
| Example | 0 h | 4 h | 24 h | 72 h | 168 h |
| ADC-C1 | 0.008 | 0.015 | 0.072 | 0.195 | 0.283 |
| ADC-C10 | 0.063 | 0.081 | 0.184 | 0.290 | 0.438 |
| ADC-C11 | 0.08 | 0.020 | 0.039 | 0.089 | 0.171 |
| ADC-C13 | 0.012 | 0.013 | 0.022 | 0.040 | 0.100 |
| ADC-C14 | BLQ | BLQ | 0.066 | 0.122 | 0.233 |

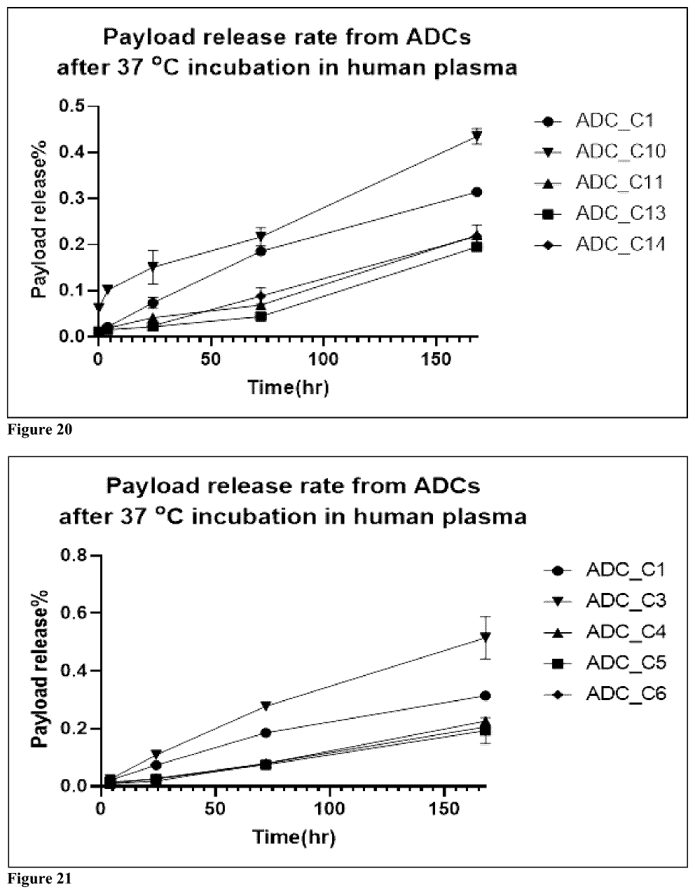

Payload release profile of ADC-C1, ADC-C10, ADC-C11, ADC-C13, and ADC-C14 can refer to FIG. 20.

All ADCs have shown comparable and acceptable payload release rate (<5% after 168 h).

### Group 2

| | DXd release rate (%) under human plasma treatment | | | | |
|---|---|---|---|---|---|
| Example | 0 h | 4 h | 24 h | 72 h | 168 h |
| ADC-C1 | 0.008 | 0.015 | 0.072 | 0.195 | 0.283 |
| ADC-C3 | BLQ | 0.015 | 0.035 | 0.082 | 0.153 |
| ADC-C4 | BLQ | 0.012 | 0.029 | 0.063 | 0.220 |
| ADC-C5 | BLQ | 0.015 | 0.025 | 0.048 | 0.133 |
| ADC-C6 | BLQ | 0.018 | 0.026 | 0.049 | 0.142 |

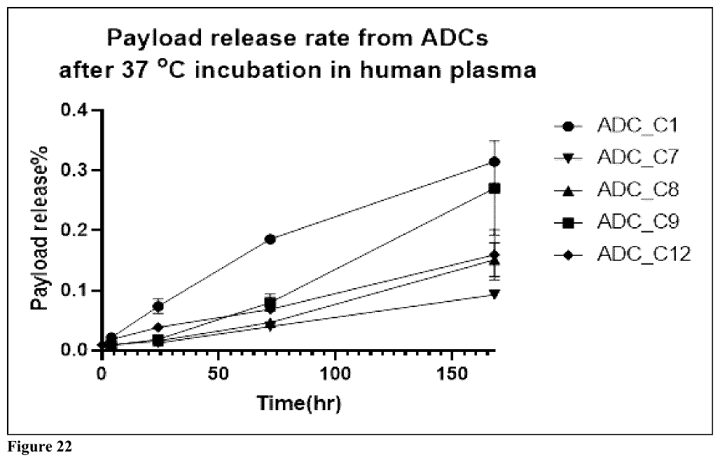

Payload release profile of ADC-C1, ADC-C3, ADC-C4, ADC-C5, and ADC-C6 can refer to FIG. 21.

All ADCs have shown comparable and acceptable payload release rate (<5% after 168 h).

### Group 3

| | DXd release rate (%) under human plasma treatment | | | | |
|---|---|---|---|---|---|
| Example | 0 h | 4 h | 24 h | 72 h | 168 h |
| ADC-C1 | 0.008 | 0.015 | 0.072 | 0.195 | 0.283 |
| ADC-C7 | BLQ | BLQ | 0.015 | 0.051 | 0.130 |
| ADC-C8 | BLQ | 0.008 | 0.031 | 0.066 | 0.108 |
| ADC-C9 | 0.063 | 0.081 | 0.184 | 0.290 | 0.438 |
| ADC-C12 | 0.010 | 0.016 | 0.056 | 0.103 | 0.144 |

Payload release profile of ADC-C1, ADC-C7, ADC-C8, ADC-C9, and ADC-C12 can refer to FIG. 22.

All ADCs have shown comparable and acceptable payload release rate (<5% after 168 h).

### Methods for Efficacy in H1975 Model Updated Models

Female BALB/c nude mice were subcutaneously implanted with 5×10⁶ H1975 cells per 200 μL PBS/matrigel in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 100-200 mm³ in size, mice were randomly allocated into groups, and were intravenously treated with vehicle or ADCs at 3 or 10 mg/kg once weekly, respectively. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

treated t=treated tumor volume at time t
treated t₀=treated tumor volume at time 0
placebo t=placebo tumor volume at time t
placebo t₀=placebo tumor volume at time 0

### Methods for Efficacy in H1650 Model

Female BALB/c nude mice were subcutaneously implanted with 3×10⁶ H1650 cells per 200 μL PBS/matrigel in the right flank. When tumors reached a mean volume of approximately 100-200 mm³ in size, mice were randomly allocated into groups, and were intravenously treated with vehicle, ADCs at 10 mg/kg once weekly, respectively.

### Methods for Efficacy in H441 Model

Female NOG mice were subcutaneously implanted with 5×10⁶ H441 cells per 300 μL PBS/matrigel in the right flank. When tumors reached a mean volume of approximately 100-200 mm³ in size, mice were randomly allocated into groups, and were intravenously treated with vehicle, ADCs at 10 mg/kg once weekly, respectively.

### In Mice PK Study

Blood samples were collected at 0, 2, 4, 8, 24, 72, and 168 h after 10 mg/kg intravenously administration of ADCs from A375 tumor bearing mice, followed by centrifugation (4° C., 3000×g, 7 min) to separate plasma. The concentrations of ADCs and total Abs were measured by in-house developed Meso Scale Discovery (MSD) ligand binding methods. Briefly, His-tagged B7H3 extracellular domain fusion protein was used as a capture reagent, biotin labelled anti-DXd Ab, or goat anti-human kappa Ab were used as the detection reagents for ADCs or total Ab measurement, respectively.

The antibodies used herein were prepared according to conventional methods, for example, vector construction, eukaryotic cell transfection such as HEK293 cell (Life Technologies Cat. No. 11625019) transfection, purification and expression. The sequences of the antibodies used herein can be found as provided herein.

TABLE 2
Platform Examples

| Compd | |
|---|---|
| No. | Linker-payload Structure |
| 6 | 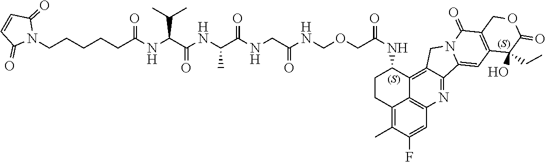 |
| | 6 |
| 7 | 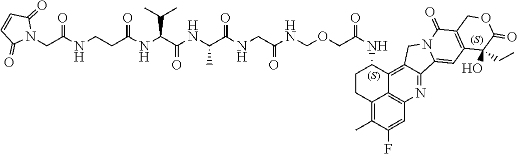 |
| | 7 |
| 8 | 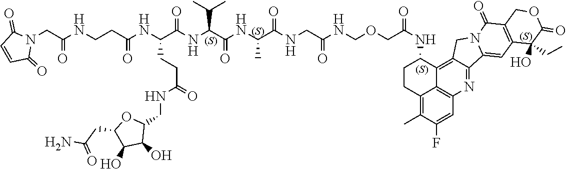 |
| | 8 |
| 9 | 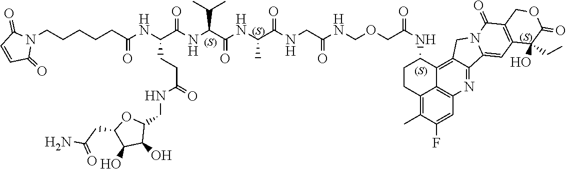 |
| | 9 |
| 10 | 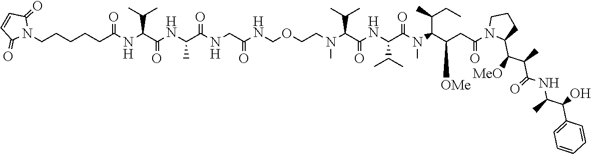 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)CCOCNC(=O)CNC(=O)[C@H](C)NC(=O)[C@@H](NC(=O)CCCCCN1C(=O)C=CC1=O)C(C)C)C(C)C] |
| | 10 |
| 11 | 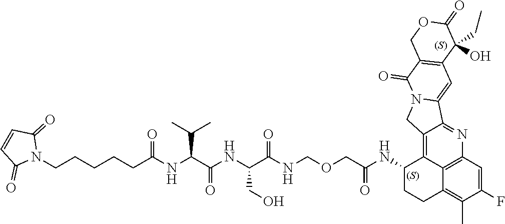 |
| | 11 |
| 12 | 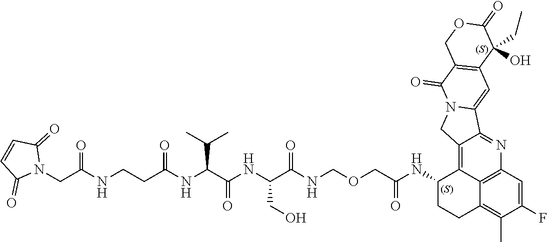 |
| | 12 |
| 13 | 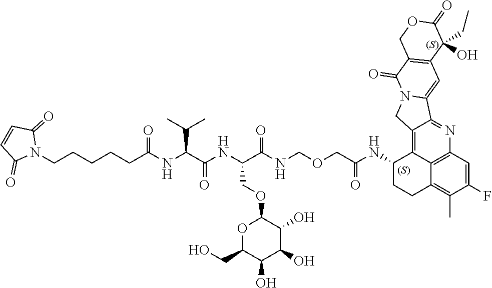 |
| | 13 |
| 14 | 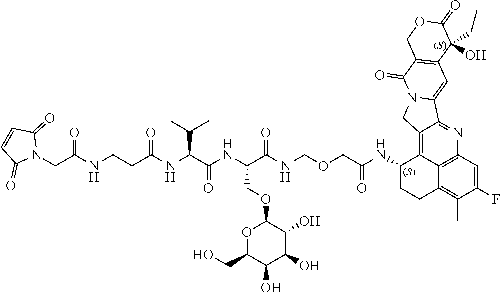 |
| | 14 |
| 15 | 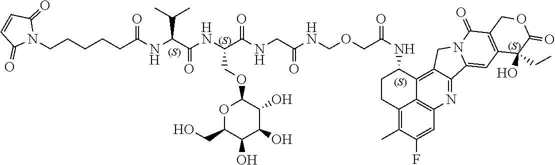 |
| | 15 |
| 16 | 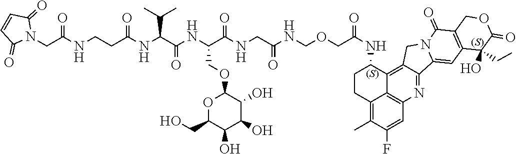 |
| | 16 |
| 17 | 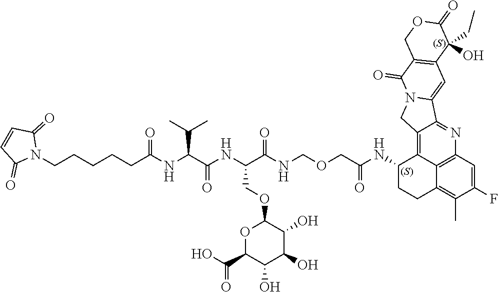 |
| | 17 |
| 18 | 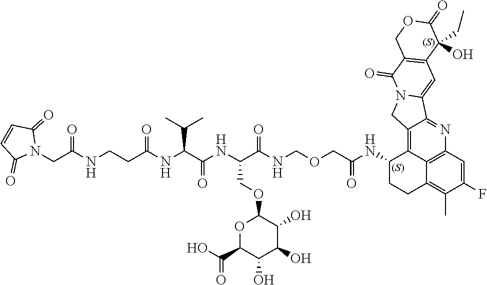 |
| | 18 |
| 19 | 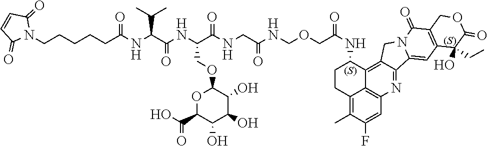 |
| | 19 |
| 20 | 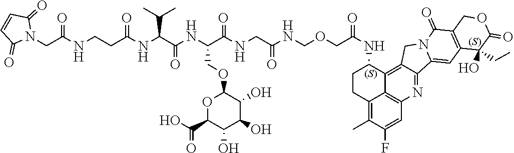 |
| | 20 |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

What is claimed is:

1. A compound of Formula (I): 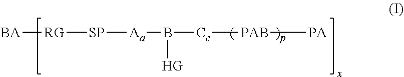
or a pharmaceutically acceptable salt, tautomer, solvate, stereoisomer, enantiomer, isotopologue, or prodrug thereof,
wherein BA is a binding agent selected from a humanized, chimeric, or human antibody or an antigen binding fragment thereof;
RG is a reactive group residue; SP is a spacer group residue; each of A, B and C is independently, in each instance, an amino acid residue; HG is a hydrophilic residue or hydrogen; subscripts a, c, and p are independently, in each instance, 0 or 1; PA is a payload residue; subscript x is from 1 to 15; and
PAB represents —NH—CH₂—O—.

2. A compound of claim 1, wherein the compound is a compound of Formula (Ia): 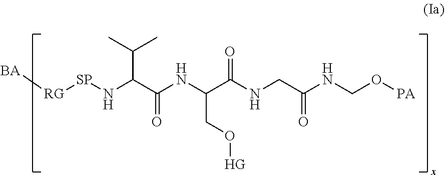
or a pharmaceutically acceptable salt, tautomer, solvate, stereoisomer, enantiomer, isotopologue, or prodrug thereof.

3. A compound of claim 1, wherein the compound is a compound of Formula (Ib): 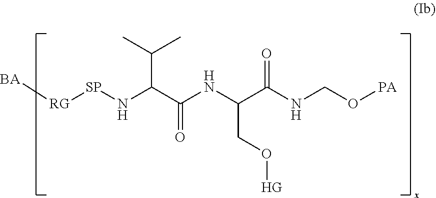
or a pharmaceutically acceptable salt, tautomer, solvate, stereoisomer, enantiomer, isotopologue, or prodrug thereof.

4. The compound of any one of claims 1-3, wherein the antibody is Ifinatamab, cofetuzumab, patritumab, or trastuzumab, or the antigen binding fragment of Ifinatamab, cofetuzumab, patritumab, or trastuzumab.

5. The compound of any one of claims 1-3, wherein the antibody is a humanized, chimeric, or human antibody or the antigen binding fragment thereof which binds to one or more of receptors selected from the group consisting of: HER2, HER3, CD7, CD19, CD20, CD22, CD25, CD27, CD30, CD33, CD37, CD38, CD46, CD70, CD71, CD74, CD79b, CD123, CD138, CD142, CD166, CD205, CD228, CCR2, CA6, p-Cadherin, CEA, CEACAM5, C4.4a, DLL3, EGFR, EGFRVIII, ENPP3, EphA2, EphrinA, FLOR1, FGFR2, GCC, cKIT, LIV1, LY6E, MSLN, MUC16, NaPi2b, Nectin4, gpNMB, PSMA, SLITRK6, STEAP1, TROP2, 5T4, SSEA4, GloboH, Gb5, STn, Tn, B7H3, BCMA, MUC1, cMet, ROR1 MSLN, FRa, CLDN18.2, CLDN6, PTK7 and Axl.

6. The compound of any one of claims 1-3, wherein the antibody is a humanized, chimeric, or human antibody or the antigen binding fragment thereof which binds to one or more of receptors selected from the group consisting of: B7H3, MUC1, FGFR2b, CLL1, CCR7,_GPC1 and GPC3.

7. The compound of any one of claims 1-6, wherein RG is 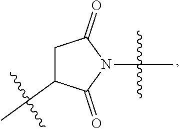 [SMILES: *C1CC(=O)N(*)C1=O]
-(Succinimid-3-yl-N)—,

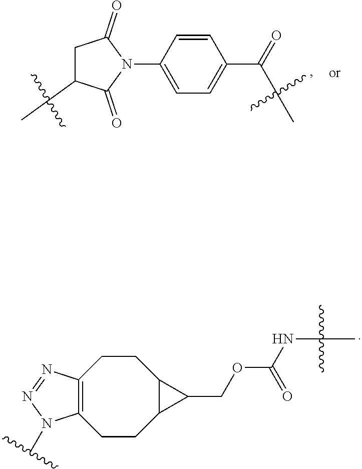 [SMILES: *C(=O)c1ccc(N2C(=O)CC(*)C2=O)cc1 ; *NC(=O)OCC1C2CCc3nnn(*)c3CCC21]

8. The compound of any one of claims 1-6, wherein RG is 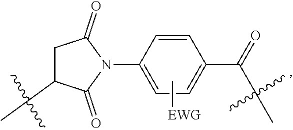
wherein EWG is an electrowithdrawn group selected from —CN, halogen, —CF₃, —C(═O)OR¹, and —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

9. The compound of any one of claims 1-6, wherein RG is

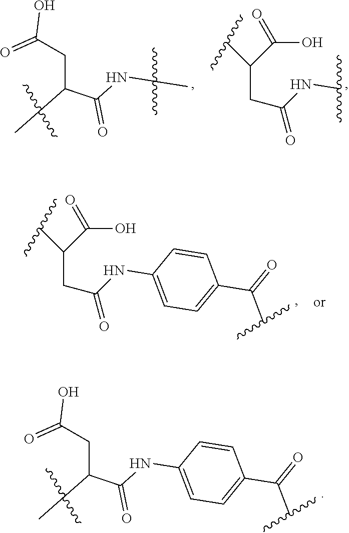 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1]

10. The compound of any one of claims 1-6, wherein RG is 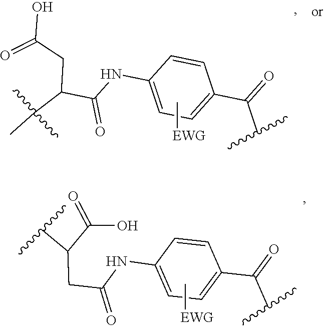
wherein EWG is an electrowithdrawn group selected from —CN, halogen, —CF₃, —C(═O)OR¹, and —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

11. A compound of Formula (II): 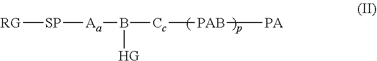
or a pharmaceutically acceptable salt, tautomer, solvate, stereoisomer, enantiomer, isotopologue, or prodrug thereof,
wherein RG is a reactive group residue; SP is a spacer group residue; each of A, B and C is independently, in each instance, an amino acid residue; HG is a hydrophilic residue or hydrogen; subscripts a, c, and p are independently, in each instance, 0 or 1; PA is a payload residue; and
PAB represents —NH—CH₂—O.

12. A compound of claim 11, wherein the compound is a compound of Formula (IIa): 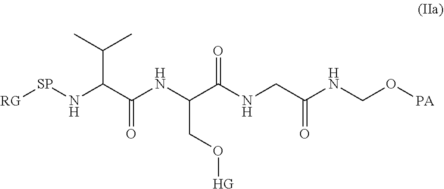
or a pharmaceutically acceptable salt, tautomer, solvate, stereoisomer, enantiomer, isotopologue, or prodrug thereof.

13. A compound of claim 11, wherein the compound is a compound of Formula (IIb): 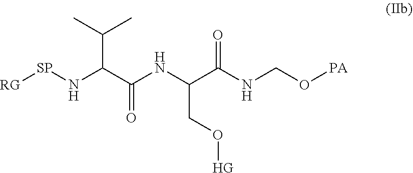
or a pharmaceutically acceptable salt, tautomer, solvate, stereoisomer, enantiomer, isotopologue, or prodrug thereof.

14. The compound of any one of claims 11-13, wherein RG is 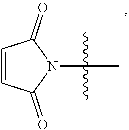 [SMILES: *N1C(=O)C=CC1=O]
Succinimid-N—,

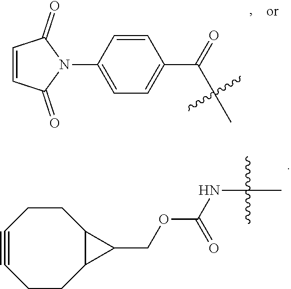 [SMILES: *C(=O)c1ccc(N2C(=O)C=CC2=O)cc1 ; *NC(=O)OCC1C2CCC#CCCC21]

15. The compound of any one of claims 11-13, wherein RG is 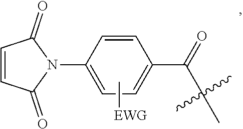
wherein EWG is an electrowithdrawn group selected from the group consisting of —CN, halogen, —CF₃, —C(═O)OR¹, and —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

16. The compound of any one of claims 11-13, wherein RG is

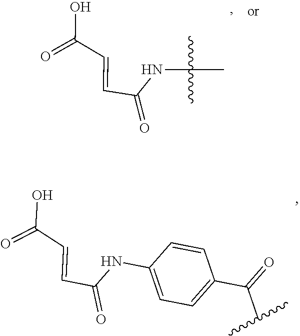 [SMILES: *NC(=O)/C=C/C(=O)O ; *C(=O)c1ccc(NC(=O)/C=C/C(=O)O)cc1]

17. The compound of any one of claims 11-13, wherein RG is 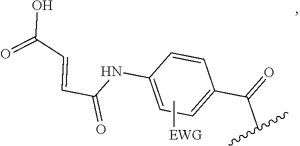
wherein EWG is an electrowithdrawn group selected from —CN, halogen, —CF₃, —C(═O)OR¹, and —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

18. The compound of any one of claims 11-17, wherein SP is —(CH₂)ₙ—C(═O)—, —CH₂—C(═O)—NH—(CH₂)ₙ—C(═O)—, —(CH₂CH₂O)ₙ—CH₂CH₂—C(═O)—, —CH[—(CH₂)ₙ—COOH]—C(═O)—, —CH₂—C(═O)—NH—(CH₂)ₙ—C(═O)—NH—(CH₂)ₙ—C(═O)—, or —C(═O)—(CH₂)ₙ—C(═O)—, wherein each of n independently represents an integer of 1 to 8.

19. The compound of any one of claims 1-18, wherein SP is —(CH₂)ₙ—C(═O)—, or —CH₂—C(═O)—NH—(CH₂)ₙ—C(═O)—; and each of n independently represents an integer of 5 or 2.

20. The compound of any one of claims 1-7, wherein RG is 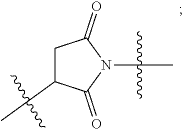 [SMILES: *C1CC(=O)N(*)C1=O]
SP is —(CH₂)ₙ—C(═O)—; and n is an integer of 1 2, 3, 4, or 5; preferably n is an integer of 1, or 2.

21. The compound of any one of claims 11-14, wherein RG is 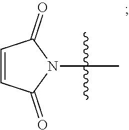 [SMILES: *N1C(=O)C=CC1=O]
SP is —(CH₂)ₙ—C(═O)—; and n is an integer of 1 2, 3, 4, or 5; preferably n is an integer of 1, or 2.

22. The compound of any one of claims 1-21, wherein HG is selected from the group consisting of a saccharide, phosphate ester, sulfate ester, a phosphodiester and a phosphonate.

23. The compound of claim 22, wherein the saccharide is selected from the group consisting of β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid or any analogue or modification thereof, or sulfate, phosphate, carboxyl, amino, or O-acetyl modification thereof; preferably β-D-galactose and β-D-glucuronic acid or a stereoisomer, enantiomer, isotopologue, or prodrug thereof.

24. The compound of claim 23, wherein HG is 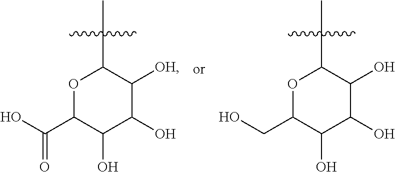 [SMILES: *C1OC(C(=O)O)C(O)C(O)C1O ; *C1OC(CO)C(O)C(O)C1O]
or a stereoisomer, enantiomer, isotopologue, or prodrug thereof.

25. The compound of any one of claims 1-24, wherein each PA independently represents a chromophore functional group.

26. The compound of claim 25, wherein each chromophore functional group is independently a functional group selected from a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; and a class or subclass of luciferin compounds.

27. The compound of any one of claims 1-24, wherein each PA is independently a cytotoxic agent, wherein the cytotoxic agent contains at least one hydroxyl group.

28. The compound of any one of claims 1-24, wherein each PA is independently selected from the group consisting of kinesin spindle protein (KSP) inhibitor (KSPi), camptothecins (such as DXd, 7-Ethyl-10-hydroxy-camptothecin (SN-38) and other analogues), Eribulin, PNU-159682, anthracyclines, nicotinamide phosphoribosyltransferase inhibitors (NAMPTi), and amatoxins.

29. The compound of any one of claims 1-24, wherein each PA independently represents formula (VI): 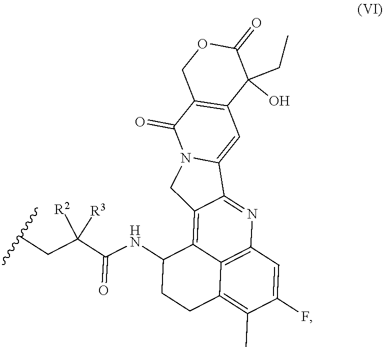
wherein each of R², and R³ is independently hydrogen, halogen, or substituted or unsubstituted C₁₋₄ alkyl.

30. The compound of any one of claims 1-24, wherein each PA independently represents

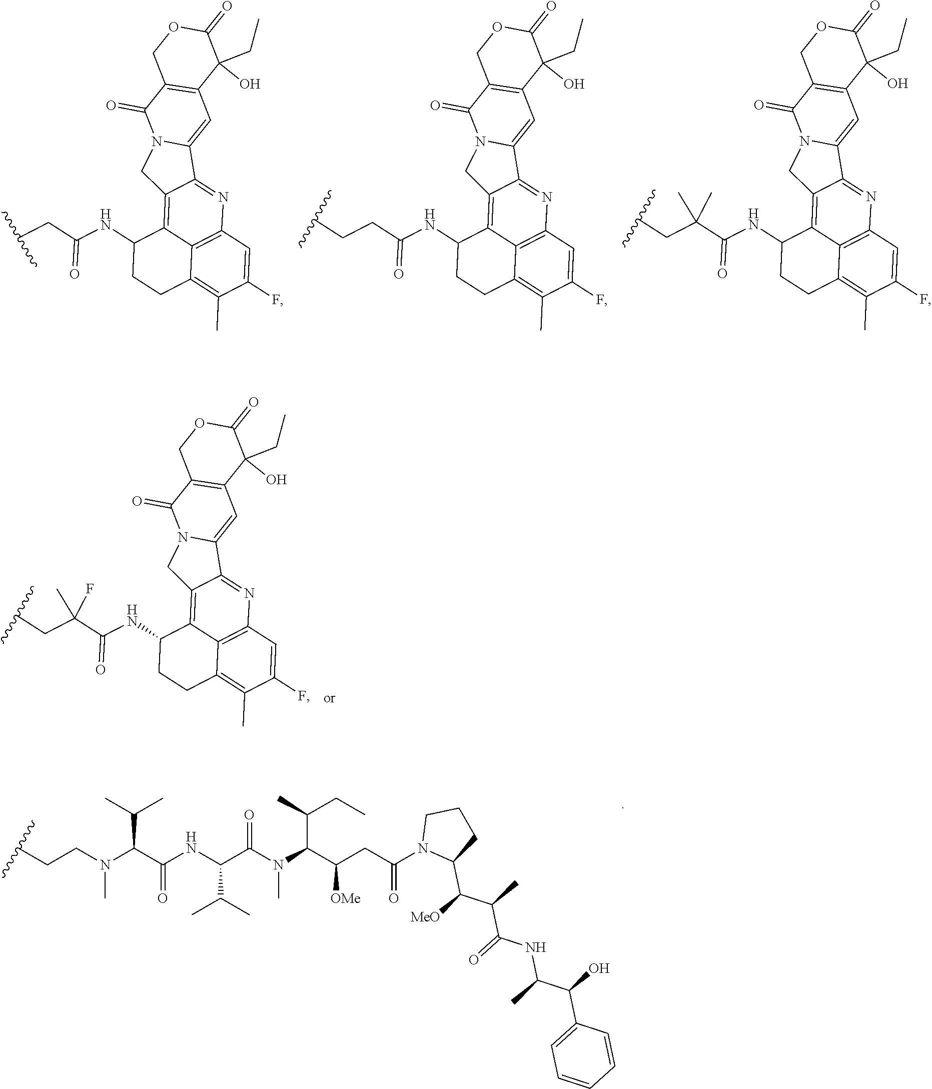 [SMILES: *CC(=O)NC1CCc2c(C)c(F)cc3nc4c(c1c23)Cn1c-4cc2c(c1=O)COC(=O)C2(O)CC ; *CCC(=O)NC1CCc2c(C)c(F)cc3nc4c(c1c23)Cn1c-4cc2c(c1=O)COC(=O)C2(O)CC ; *CC(C)(C)C(=O)NC1CCc2c(C)c(F)cc3nc4c(c1c23)Cn1c-4cc2c(c1=O)COC(=O)C2(O)CC ; *CC(C)(F)C(=O)N[C@H]1CCc2c(C)c(F)cc3nc4c(c1c23)Cn1c-4cc2c(c1=O)COC(=O)C2(O)CC ; *CCN(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

31. The compound of any one of claims 1-10 and 18-30, wherein the compound is selected from the group consisting of the compounds in Table 1.

32. The compound of any one of claims 11-30, wherein the compound is selected from the group consisting of the compounds in Table 2.

33. A ligand-drug conjugate or a pharmaceutically acceptable salt or solvate thereof, wherein the ligand-drug conjugate comprises a structure of formula (III): 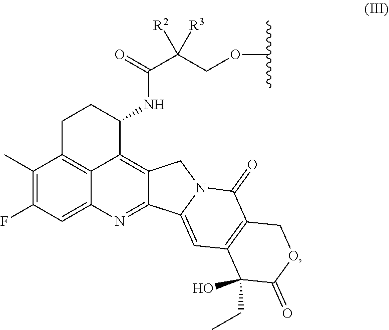
wherein
each of R² and R³ is, independently, hydrogen, halogen, or alkyl.

34. A pharmaceutical composition comprising a compound of any one of claims 1-10, 18-31, and 33, or a pharmaceutically acceptable salt, tautomer, solvate, stereoisomer, enantiomer, isotopologue, or prodrug thereof, and a pharmaceutically acceptable excipient.

35. A method of treating a proliferative disease, a metabolic disease, inflammation, or a neurodegenerative disease in a subject comprising administering to the subject an effective treatment amount of a compound of any one of claims 1-10, 18-31, and 33, or a pharmaceutical composition of claim 34.
